(19) United States
(12) Patent Application Publication
(10) Pub. No.: US 2017/0370954 A1
(43) Pub. Date: Dec. 28, 2017

(54) Biomarkers for Fatty Liver Disease and Methods Using the Same

(71) Applicant: Metabolon, Inc., Morrisville, NC (US)

(72) Inventors: Regis Perichon, Cary, NC (US); Lauren Nicole Bell, Durham, NC (US); Jacob Wulff, Morrisville, NC (US); Uyen Thao Nguyen, Sacramento, CA (US); Steven M. Watkins, Davis, CA (US)

(21) Appl. No.: 15/527,362

(22) PCT Filed: Nov. 18, 2015
(86) PCT No.: PCT/US15/61215

§ 371 Date: May 17, 2017

Related U.S. Application Data
(60) Provisional application No. 62/081,903, filed on Nov. 19, 2014.
(60) Provisional application No. 62/141,494, filed on Apr. 1, 2015.

(51) Int. Cl. G01N 33/92; G01N 33/82; G01N 33/50; G01N 33/68

(52) U.S. Cl. CPC G01N 33/92; G01N 33/6806; G01N 33/82; G01N 33/50; G01N 2560/00; G01N 2800/085; G01N 2800/56; G01N 2800/7052; G01N 2570/00

(57) ABSTRACT

Biomarkers of NASH, NAFLD, and fibrosis and methods for diagnosis (or aiding in the diagnosis) of NAFLD, NASH and/or fibrosis are described herein. Additionally, methods of distinguishing between NAFLD and NASH, methods of classifying the stage of fibrosis, methods of determining the severity of liver disease, methods of determining the severity of liver disease or fibrosis, and methods of monitoring progression/regression of NASH, NAFLD, and/or fibrosis are described herein.

### CROSS-REFERENCE TO RELATED APPLICATIONS

This application claims the benefit of U.S. Provisional Patent Application No. 62/081,903, filed Nov. 19, 2014, and U.S. Provisional Patent Application

No. 62/141,494, filed Apr. 1, 2015, the entire contents of which are hereby incorporated herein by reference.

### FIELD

The invention generally relates to biomarkers for fatty liver disease and methods based on the same biomarkers.

### BACKGROUND

The prevalence of Nonalcoholic Fatty Liver Disease (NAFLD), which encompasses an entire histologic spectrum ranging from simple, benign hepatic steatosis to nonalcoholic steatohepatitis (NASH) characterized by lipid accumulation, inflammation, hepatocyte ballooning, and varying degrees of fibrosis, continues to increase in concert with the obesity epidemic. Despite increasing awareness of obesity-related liver disease, the pathogenesis of NAFLD and NASH is poorly understood and there are no FDA-approved therapies with NASH as an indication. Diagnosis of NASH remains complicated and with significant risk due to the requirement for an invasive liver biopsy. Therefore, identification of a profile of blood-based metabolite biomarkers able to diagnose and stage NAFLD in a patient with or suspected of having liver disease for prognostic purposes (i.e., at risk of progression to a more advanced liver disease stage) is a significant unmet medical need.

Fatty change in the liver results from excessive accumulation of lipids within hepatocytes. Fatty liver is the accumulation of triglycerides and other fats in the liver cells. Fatty liver disease can range from fatty liver alone (simple fatty liver, steatosis) to fatty liver associated with hepatic inflammation (steatohepatitis). Although having fat in the liver is not normal, by itself it probably causes little harm or permanent damage. Steatosis is generally believed to be a benign condition, with rare progression to chronic liver disease. In contrast, steatohepatitis may progress to liver fibrosis and cirrhosis, can be associated with hepatocellular carcinoma and may result in liver-related morbidity and mortality.

Steatosis can occur with the use of alcohol (alcohol-related fatty liver) or in the absence of alcohol (nonalcoholic fatty liver disease, NAFLD). Steatohepatitis may be related to alcohol-induced hepatic damage or may be unrelated to alcohol. If steatohepatitis is present but a history of alcohol use is not, the condition is termed nonalcoholic steatohepatitis (NASH).

In the absence of alcohol the main risk factors for simple fatty liver (NAFLD) and NASH are obesity, diabetes, and high triglyceride levels. In NASH, fat builds up in the liver and eventually causes scar tissue. This type of hepatitis appears to be associated with diabetes, protein malnutrition, obesity, coronary artery disease, and treatment with corticosteroid medications. Fibrosis or cirrhosis in the liver is present in 15-50% of patients with NASH. Approximately 30% of patients with fibrosis develop cirrhosis after 10 years.

Fatty liver disease is now the most common cause for elevated liver function tests in the United States. It is now probably the leading reason for mild elevations of transaminases. Steatosis affects approximately 25-35% of the general population. NAFLD is found in over 80% of patients who are obese. NASH affects 2 to 5 percent of Americans and has been detected in 1.2-9% of patients undergoing routine liver biopsy. Over 50% of patients undergoing bariatric surgery have NASH. The disease strikes males and females; early studies report >70% of cases were in females but recent studies report 50% of patients are females. Fatty liver occurs in all age groups. In the United States NASH is the most common liver disease among adolescents and is the third most common cause of chronic liver disease in adults (after hepatitis C and alcohol).

Both NASH and NAFLD are becoming more common, possibly because of the greater number of Americans with obesity. In the past 10 years, the rate of obesity has doubled in adults and tripled in children. Obesity also contributes to diabetes and high blood cholesterol, which can further complicate the health of someone with NASH. Diabetes and high blood cholesterol are also becoming more common among Americans.

NASH is usually a silent disease with few or no symptoms. Patients generally feel well in the early stages and only begin to have symptoms—such as fatigue, weight loss, and weakness—once the disease is more advanced or cirrhosis develops. The progression of NASH can take years, even decades. The process can stop and, in some cases, reverse on its own without specific therapy. Or NASH can slowly worsen, causing scarring or “fibrosis” to appear and accumulate in the liver. As fibrosis worsens, cirrhosis develops; the liver becomes seriously scarred, hardened, and unable to function normally. Not every person with NASH develops cirrhosis, but once serious scarring or cirrhosis is present, few treatments can halt the progression. A person with cirrhosis experiences fluid retention, muscle wasting, bleeding from the intestines, and liver failure. Liver transplantation is the only treatment for advanced cirrhosis with liver failure, and transplantation is increasingly performed in people with NASH. NASH ranks as one of the major causes of cirrhosis in America, behind hepatitis C and alcoholic liver disease.

NASH is usually first suspected in a person who is found to have elevations in liver tests that are included in routine blood test panels, such as alanine aminotransferase (ALT) or aspartate aminotransferase (AST). When further evaluation shows no apparent reason for liver disease (such as medications, viral hepatitis, or excessive use of alcohol) and when x-rays or imaging studies of the liver show fat, NASH is suspected. The only means of proving a diagnosis of NASH and separating it from simple fatty liver is a liver biopsy. A liver biopsy requires a needle to be inserted through the skin and the removal of a small piece of the liver. If the tissue shows fat without inflammation and damage, simple fatty liver or NAFLD is diagnosed. NASH is diagnosed when microscopic examination of the tissue shows fat along with inflammation and damage to liver cells. A biopsy is required to determine whether scar tissue has developed in the liver. Currently, no blood tests or scans can reliably provide this information. Therefore there exists a need for a less invasive diagnostic method (i.e. a method that would not require a biopsy).

### SUMMARY

In one aspect, the present disclosure provides methods of diagnosing or aiding in the diagnosis of liver disease in a subject, comprising: analyzing a biological sample from said subject to determine the level(s) of one or more biomarkers for liver disease in the sample, where the one or more biomarkers are selected from Tables 12, 2, 3, 4, 5, 7, 8, 10, 11, 14, 16 and/or 18 and comparing the level(s) of the one or more biomarkers in the sample to liver disease-positive and/or liver disease-negative reference levels of the one or more biomarkers in order to diagnose whether the subject has liver disease.

In another aspect, the present disclosure provides methods of diagnosing or aiding in the diagnosis of NASH in a subject, comprising: analyzing a biological sample from said subject to determine the level(s) of one or more biomarkers for NASH in the sample, where the one or more biomarkers are selected from Tables 7, 8, 10 and/or 11 and comparing the level(s) of the one or more biomarkers in the sample to NASH-positive and/or NASH-negative reference levels of the one or more biomarkers in order to diagnose whether the subject has NASH.

In a further aspect, the disclosure provides methods of diagnosing or aiding in the diagnosis of NAFLD in a subject, comprising: analyzing a biological sample from said subject to determine the level(s) of one or more biomarkers for NAFLD in the sample, where the one or more biomarkers are selected from Tables 2, 3, 4, 5, 7, 8, 10, and/or 11; and comparing the level(s) of the one or more biomarkers in the sample to NAFLD-positive and/or NAFLD-negative reference levels of the one or more biomarkers in order to diagnose whether the subject has NAFLD. In a feature of this aspect, the one or more biomarkers may be selected from the group consisting of 5-methylthioadenosine (5-MTA), glycine, serine, leucine, 4-methyl-2-oxopentanoate, 3-methyl-2-oxovalerate, valine, 3-methyl-2-oxobutyrate, 2-hydroxybutyrate, prolylproline, lanosterol, tauro-beta-muricholate, and deoxycholate.

In another aspect, the disclosure provides methods of distinguishing NASH from NAFLD in a subject, comprising analyzing a biological sample from said subject to determine the level(s) of the one or more biomarkers for NASH and/or NAFLD in the sample where the one or more biomarkers are selected from Tables 2, 3, 4, 5, 7, 8, 10, and/or 11 and comparing the level(s) of the one or more biomarkers in the sample to reference levels of the one or more biomarkers in order to distinguish NASH from NAFLD.

In another aspect, the disclosure provides methods of diagnosing or aiding in the diagnosis of liver fibrosis in a subject, comprising analyzing a biological sample from said subject to determine the level(s) of one or more biomarkers for fibrosis in the sample, where the one or more biomarkers are selected from Tables 12, 10, 11, 14, 16, and/or 18 and comparing the level(s) of the one or more biomarkers in the sample to fibrosis-positive and/or fibrosis-negative reference levels of the one or more biomarkers in order to diagnose whether the subject has fibrosis.

In another aspect, the disclosure provides methods of determining the stage of fibrosis of a subject having liver fibrosis, comprising analyzing a biological sample from said subject to determine the level(s) of one or more biomarkers for liver disease in the sample, wherein the one or more biomarkers are selected from Tables 12, 10, 11, 14, 16 and/or 18, and comparing the level(s) of the one or more biomarkers in the sample to the liver fibrosis stage reference levels of the one or more biomarkers in order to determine the stage of the liver fibrosis.

In another embodiment, the disclosure provides methods of monitoring the progression/regression of liver disease in a subject, comprising analyzing a first biological sample from said subject to determine the level(s) of one or more biomarkers for liver disease in the sample, wherein the one or more biomarkers are selected from Tables 12, 2, 3, 4, 5, 7, 8, 10, 11, 14, 16, and/or 18 and the first sample is obtained from said subject at a first time point; analyzing a second biological sample from said subject to determine the level(s) of the one or more biomarkers, wherein the second sample is obtained from said subject at a second time point; and comparing the level(s) of one or more biomarkers in the first sample to the level(s) of the one or more biomarkers in the second sample in order to monitor the progression/regression of liver disease in the subject.

In a further embodiment, the disclosure provides methods of distinguishing less severe from more severe in a subject having, comprising analyzing a biological sample from said subject to determine the level(s) of one or more biomarkers for in the sample, wherein the one or more biomarkers are selected from Tables 12, 2, 3, 4, 5, 7, 8, 10, 11, 14, 16, and/or 18, and comparing the level(s) of the one or more biomarkers in the sample to less severe and/or more severe reference levels of the one or more biomarkers in order to determine the severity of the subject's liver disease.

In yet another aspect of the invention, a method of diagnosing or aiding in diagnosing whether a subject has liver disease comprises analyzing a biological sample from a subject to determine the level(s) of one or more biomarkers for liver disease in the sample, wherein the one or more biomarkers are selected from Tables 19 and 20, and comparing the level(s) of the one or more biomarkers in the sample to liver disease-positive and/or liver disease-negative reference levels of the one or more biomarkers in order to diagnose whether the subject has liver disease.

In a feature of this aspect, the liver disease may be NASH and the one or more biomarkers may be selected from Table 19. In another feature of this aspect, the liver disease may be fibrosis and the one or more biomarkers may be selected from Table 20. In further features, the diagnosis may comprise distinguishing NASH from NAFLD or distinguishing NASH from fibrosis.

In a further aspect of the invention, a method of determining the fibrosis stage of a subject having liver fibrosis comprises analyzing a biological sample from a subject to determine the level(s) of one or more biomarkers for liver disease in the sample, wherein the one or more biomarkers are selected from Table 20, and comparing the level(s) of the one or more biomarkers in the sample to high stage liver fibrosis and/or low stage liver fibrosis reference levels of the one or more biomarkers in order to determine the stage of the liver fibrosis.

In an additional aspect of the invention, a method of monitoring progression/regression of liver disease in a subject comprises analyzing a first biological sample from a subject to determine the level(s) of one or more biomarkers for liver disease in the sample, wherein the one or more biomarkers are selected from Tables 19 and/or 20 and the first sample is obtained from the subject at a first time point; analyzing a second biological sample from a subject to determine the level(s) of the one or more biomarkers, wherein the second sample is obtained from the subject at a second time point; and comparing the level(s) of one or more biomarkers in the first sample to the level(s) of the one or more biomarkers in the second sample in order to monitor the progression/regression of liver disease in the subject.

In another aspect of the invention, a method of distinguishing less severe liver disease from more severe liver disease in a subject having liver disease comprises analyzing a biological sample from a subject to determine the level(s) of one or more biomarkers for liver disease in the sample, wherein the one or more biomarkers are selected from Tables 19 and/or 20, and comparing the level(s) of the one or more biomarkers in the sample to less severe liver disease and/or more severe liver disease reference levels of the one or more biomarkers in order to determine the severity of the subject's liver disease.

In yet another aspect, a method of aiding in distinguishing NASH from NAFLD in a subject having been diagnosed with a liver disease comprises analyzing a biological sample from a subject to determine the level(s) of one or more biomarkers for liver disease in the sample, wherein the one or more biomarkers are selected from Table 19, and comparing the level(s) of the one or more biomarkers in the sample to liver disease reference levels of the one or more biomarkers in order to distinguish between NASH and NAFLD in the subject.

In a further aspect, a method of aiding in distinguishing NASH from fibrosis in a subject having been diagnosed with a liver disease comprises analyzing a biological sample from a subject to determine the level(s) of one or more biomarkers for liver disease in the sample, wherein the one or more biomarkers are selected from Table 19 and/or 20, and comparing the level(s) of the one or more biomarkers in the sample to liver disease reference levels of the one or more biomarkers in order to distinguish between NASH and fibrosis in the subject.

In yet another embodiment, the disclosure provides methods of determining a Liver Disease Score.

### BRIEF DESCRIPTION OF THE DRAWINGS

FIG. 1 is a graphical illustration showing mean R-square values (Y-axis) of MRI PDFF correlation as a function of the number of metabolites (X-axis).

FIG. 2 is a graphical illustration showing range of calculated areas under the curve (AUC) for separating fibrosis stage 0-1 from fibrosis stage 2-4 by fitting all possible model combinations for the eight metabolites with an AUC>0.6663.

FIG. 3 is a graphical illustration showing range of calculated areas under the curve (AUC) for separating fibrosis stage 0-1 from fibrosis stage 3-4 by fitting all possible model combinations for the seven metabolites with an AUC>0.7217.

### DETAILED DESCRIPTION

Biomarkers of NASH, NAFLD, and fibrosis, methods for diagnosis (or aiding in the diagnosis) of NAFLD, NASH and/or fibrosis, methods of distinguishing between NAFLD and NASH, methods of classifying the stage of fibrosis, methods of determining the severity of liver disease, methods of determining the severity of liver disease or fibrosis, methods of monitoring progression/regression of NASH, NAFLD, and/or fibrosis, as well as other methods based on biomarkers of liver disease are described herein.

Prior to describing this invention in further detail, however, the following terms will first be defined.

### Definitions:

“Biomarker” means a compound, preferably a metabolite, that is differentially present (i.e., increased or decreased) in a biological sample from a subject or a group of subjects having a first phenotype (e.g., having a disease) as compared to a biological sample from a subject or group of subjects having a second phenotype (e.g., not having the disease). A biomarker may be differentially present at any level, but is generally present at a level that is increased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent). A biomarker is preferably differentially present at a level that is statistically significant (i.e., a p-value less than 0.05 and/or a q-value of less than 0.10 as determined using either Welch's T-test or Wilcoxon's rank-sum Test).

The “level” of one or more biomarkers means the absolute or relative amount or concentration of the biomarker in the sample.

“Sample” or “biological sample” means biological material isolated from a subject. The biological sample may contain any biological material suitable for detecting the desired biomarkers, and may comprise cellular and/or non-cellular material from the subject. The sample can be isolated from any suitable biological fluid such as, for example, blood, blood plasma, blood serum, urine, or cerebral spinal fluid (CSF).

“Subject” means any animal, but is preferably a mammal, such as, for example, a human, monkey, non-human primate, mouse, or rabbit.

A “reference level” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. For example, a “NASH-positive reference level” of a biomarker means a level of a biomarker that is indicative of a positive diagnosis of NASH in a subject, and a “NASH-negative reference level” of a biomarker means a level of a biomarker that is indicative of a negative diagnosis of NASH in a subject. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in biological samples (e.g., LC-MS, GC-MS, etc.), where the levels of biomarkers may differ based on the specific technique that is used.

“Non-biomarker compound” means a compound that is not differentially present in a biological sample from a subject or a group of subjects having a first phenotype (e.g., having a first disease) as compared to a biological sample from a subject or group of subjects having a second phenotype (e.g., not having the first disease). Such non-biomarker compounds may, however, be biomarkers in a biological sample from a subject or a group of subjects having a third phenotype (e.g., having a second disease) as compared to the first phenotype (e.g., having the first disease) or the second phenotype (e.g., not having the first disease).

“Metabolite”, or “small molecule”, means organic and inorganic molecules which are present in a cell. The term does not include large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weights of over 2,000, 3,000, 4,000, 5,000, 6,000, 7,000, 8,000, 9,000, or 10,000). The small molecules of the cell are generally found free in solution in the cytoplasm or in other organelles, such as the mitochondria, where they form a pool of intermediates which can be metabolized further or used to generate large molecules, called macromolecules. The term “small molecules” includes signaling molecules and intermediates in the chemical reactions that transform energy derived from food into usable forms. Examples of small molecules include sugars, fatty acids, amino acids, nucleotides, intermediates formed during cellular processes, and other small molecules found within the cell.

“Metabolic profile”, or “small molecule profile”, means a complete or partial inventory of small molecules within a targeted cell, tissue, organ, organism, or fraction thereof (e.g., cellular compartment). The inventory may include the quantity and/or type of small molecules present. The “small molecule profile” may be determined using a single technique or multiple different techniques.

“Metabolome” means all of the small molecules present in a given organism.

“Steatosis” refers to fatty liver disease without the presence of inflammation. The condition can occur with the use of alcohol or in the absence of alcohol use.

“Non-alcoholic fatty liver disease” (NAFLD) refers to fatty liver disease (steatosis) that occurs in subjects even in the absence of consumption of alcohol in amounts considered harmful to the liver.

“Steatohepatitis” refers to fatty liver disease that is associated with inflammation. Steatohepatitis can progress to cirrhosis and can be associated with hepatocellular carcinoma. The condition can occur with the use of alcohol or in the absence of alcohol use.

“Non-alcoholic steatohepatitis” (NASH) refers to steatohepatitis that occurs in subjects even in the absence of consumption of alcohol in amounts considered harmful to the liver. NASH can progress to cirrhosis and can be associated with hepatocellular carcinoma.

“Fibrosis” refers to the accumulation of extracellular matrix proteins in the liver as a result of ongoing inflammation. Fibrosis is classified histologically in a liver biopsy sample into five stages, 0-4. Stage 0 means no fibrosis, Stage 1 refers to mild fibrosis, Stage 2 refers to moderate fibrosis, Stage 3 refers to severe fibrosis, and Stage 4 refers to cirrhosis.

“Liver disease”, as used herein refers to NAFLD, NASH, fibrosis, and cirrhosis.

“NAFLD Activity Score” or “NAS” refers to a histological scoring system for NAFLD. The score is comprised of evaluation of changes in histological features such as steatosis, lobular inflammation, absence of lipogranulomas, and hepatocyte ballooning. Fibrosis is assessed independently of the NAS.

“Severity” of liver disease refers to the degree of liver disease on the spectrum of non-alcoholic liver disease activity, ranging from low severity disease associated with fat accumulation in the liver (NAFLD), with an increased severity associated with low levels of inflammation and/or fibrosis in addition to fat accumulation (i.e., borderline NASH), and a further increase in severity associated with higher levels of inflammation and fibrosis (i.e., NASH). Severity may be based on fibrosis stages or may also be assessed using the NAS.

With respect to the nomenclature for select fatty acid lipid metabolites used herein, fatty acids labeled with a prefix “CE”, “DAG”, “FFA”, “PC”, “PE”, “LPC”, “LPE”, “O-PC”, “P-PE”, “PI”, “SM”, “TAG”, “CER”, “DCER”, “LCER”, or “TL” refer to the indicated fatty acids present within cholesteryl esters, diacylglycerols (diglycerides), free fatty acids, phosphatidylcholines, phosphatidylethanolamines, lysophosphatidylcholines, lysophosphatidylethanolamines, 1-ether linked phosphatidylcholines, 1-vinyl ether linked phosphatidylethanolamines (plasmalogens), phosphoinositols, sphingomyelins, triacylglycerols (triglycerides), ceramides, dihydroceramides, lactoceramides, and total lipids, respectively, in a sample. “TL” refer to the indicated fatty acids present within total lipids in a sample. In some embodiments, the indicated fatty acid components are quantified as a proportion of the total fatty acids within the lipid class indicated by the prefix. For example, the abbreviation “TL16:0” indicates the percentage of total lipid in the sample comprised on palmitic acid (16:0). The term “TLTL” or “Total Total Lipid” indicates the absolute amount (e.g., in n Moles per gram) of total lipid present in the sample. In some embodiments, the indicated fatty acid components are quantified as a proportion of total fatty acids within the lipid class indicated by the prefix. References to fatty acids without a prefix or other indication of a particular lipid class generally indicate fatty acids present within total lipids in a sample. The term “LC” following a prefix “CE”, “DAG”, “FFA”, “PC”, “PE”, “LPC”, “LPE”, “O-PC”, “P-PE”, “PI”, “SM”, “TAG”, “CER”, “DCER”, or “LCER” refers to the amount of the total lipid class indicated by the prefix in the sample (e.g., the concentration of lipids of that class expressed as n Moles per gram of serum or plasma). For example, with respect to a measurement taken from plasma or serum, the abbreviation “PC 18:2n6” indicates the percentage of plasma or serum phosphatidylcholine comprised of linoleic acid (18:2n6), and the term “TGLC” indicates the absolute amount (e.g., in n Moles per gram) of triglyceride present in plasma or serum. For triaclyglycerols, the metabolite name refers to the parent mass of the compound (e.g., TAG53:6-FA18:2 indicates that the metabolite is a triacylglycerol with attached fatty acids having 53 total carbons and 6 total double bonds.—FA18:2 refers to the fragment identified on the mass spectrometer (i.e., one of the three fatty acids of the TAG in this example is 18:2)). “MUFA”, “PUFA”, and “SFA” refer to monounsaturated fatty acid, polyunsaturated fatty acid, and saturated fatty acid, respectively.

### I. Biomarkers

The NAFLD, NASH, and fibrosis biomarkers described herein were discovered using metabolomic profiling techniques. Such metabolomic profiling techniques are described in more detail in the Examples set forth below as well as in U.S. Pat. Nos. 7,005,255; 7,329,489; 7,550,258; 7,550,260; 7,553,616; 7,635,556; 7,682,783; 7,682,784; 7,910,301 and 7,947,453 the entire contents of which are hereby incorporated herein by reference.

Generally, metabolic profiles were determined for biological samples from human subjects diagnosed with NAFLD, NASH, or fibrosis as well as from one or more other groups of human subjects (e.g., control subjects not diagnosed with NAFLD, NASH, or fibrosis). The metabolic profile for biological samples from a subject having NAFLD, NASH, or fibrosis was compared to the metabolic profile for biological samples from the one or more other groups of subjects. Those molecules differentially present, including those molecules differentially present at a level that is statistically significant, in the metabolic profile of samples from subjects with NAFLD, NASH, or fibrosis as compared to another group (e.g., control subjects not diagnosed with NAFLD, NASH, or fibrosis) were identified as biomarkers to distinguish those groups. In addition, those molecules differentially present, including those molecules differentially present at a level that is statistically significant, in the metabolic profile of samples from subjects with NAFLD, NASH, or fibrosis as compared to another group were also identified as biomarkers to distinguish those groups.

The biomarkers are discussed in more detail herein. The biomarkers that were discovered correspond with the following group(s):

Biomarkers for distinguishing subjects having NAFLD vs. subjects not diagnosed with NAFLD (see Tables 2, 3, 4, 5);

Biomarkers for distinguishing subjects having NASH vs. subjects having NAFLD (see Tables 7, 8);

Biomarkers for distinguishing subjects having fibrosis vs. control subjects not having fibrosis (see Tables 10, 11, 12, 14, 16, 18, 20);

Biomarkers for distinguishing stages of fibrosis (see Tables 10, 11, 12, 14, 16, 18).

Biomarkers for distinguishing subjects having NASH vs. control subjects not having NASH (see Table 20)

### II. Methods

### A. Diagnosis of Liver Disease

The identification of biomarkers for NAFLD, NASH, and fibrosis allows for the diagnosis of (or aiding in the diagnosis of) liver disease in subjects presenting with one or more symptoms consistent with the presence of liver disease and includes the initial diagnosis of liver disease in a subject not previously identified as having liver disease and diagnosis of recurrence of liver disease in a subject previously treated for liver disease. A method of diagnosing (or aiding in diagnosing) whether a subject has liver disease comprises (1) analyzing a biological sample from a subject to determine the level(s) of one or more biomarkers of liver disease in the sample and (2) comparing the level(s) of the one or more biomarkers in the sample to liver disease-positive and/or liver disease-negative reference levels of the one or more biomarkers in order to diagnose (or aid in the diagnosis of) whether the subject has liver disease. The one or more biomarkers that are used are selected from Tables 2, 3, 4, 5, 7, 8, 10, 11, 12, 14, 16, 18, 19, and/or 20 and combinations thereof. When such a method is used to aid in the diagnosis of liver disease, the results of the method may be used along with other methods (or the results thereof) useful in the clinical determination of whether a subject has liver disease.

Any suitable method may be used to analyze the biological sample in order to determine the level(s) of the one or more biomarkers in the sample. Suitable methods include chromatography (e.g., HPLC, gas chromatography, liquid chromatography), mass spectrometry (e.g., MS, MS-MS), enzyme-linked immunosorbent assay (ELISA), antibody linkage, other immunochemical techniques, and combinations thereof. Further, the level(s) of the one or more biomarkers may be measured indirectly, for example, by using an assay that measures the level of a compound (or compounds) that correlates with the level of the biomarker(s) that are desired to be measured.

The levels of one or more of the biomarkers in Tables 2, 3, 4, 5, 7, 8, 10, 11, 12, 14, 16, 18, 19, and/or 20 including a combination of all of the biomarkers in Tables 2, 3, 4, 5, 7, 8, 10, 11, 12, 14, 16, 18, 19, and/or 20 and combinations thereof or any fraction thereof, may be determined and used in methods of aiding in diagnosing whether a subject has liver disease. Determining levels of combinations of the biomarkers may allow greater sensitivity and specificity in diagnosing liver disease and aiding in the diagnosis of liver disease. For example, ratios of the levels of certain biomarkers (and non-biomarker compounds) in biological samples may allow greater sensitivity and specificity in diagnosing liver disease and aiding in the diagnosis of liver disease.

In one example, the levels of one or more biomarkers in Tables 2, 3, 4, 5, 7, 8, 10, and/or 11, and any combination thereof including a combination of all of the biomarkers may be determined in the methods of diagnosing or aiding in diagnosing whether a subject has NAFLD. For example, one or more of the following biomarkers may be used alone or in combination to diagnose or aid in diagnosing NAFLD: epiandrosterone sulfate, androsterone sulfate, I-urobilinogen, 16-hydroxypalmitate, fucose, taurine, 3-hydroxydecanoate, 3-hydroxyoctanoate, 16a-hydroxy DHEA 3-sulfate, dehydroisoandrosterone sulfate (DHEA-S), 5-methylthioadenosine (MTA), gamma-glutamylhistidine, valylglycine, 3-hydroxyisobutyrate, cyclo (L-phe-L-pro), 2-aminoadipate, 4-methyl-2-oxopentanoate, 2-hydroxybutyrate, prolylproline, and tauro-beta-muricholate. In another example, one or more additional biomarkers may optionally be selected from the group consisting of: isoleucine, glutamate, alpha-ketoglutarate, TL16:1n7 (16:1n7, palmitoleic acid), TL16:0 (16:0, palmitic acid), taurocholate, glycocholate, taurochenodeoxycholate, glycochenodeoxycholate, glycine, serine, leucine, deoxycholate, 3-methyl-2-oxovalerate, valine, 3-methyl-2-oxobutyrate, and lanosterol and may be used in combination with the one or more biomarkers.

In another example, the levels of one or more biomarkers in Tables 7, 8, 10, 11 and/or 20 and any combination thereof including a combination of all of the biomarkers may be determined in the methods of diagnosing or aiding in diagnosing whether a subject has NASH. For example, one or more of the following biomarkers may be used alone or in combination to diagnose or aid in diagnosing NASH: epiandrosterone sulfate, androsterone sulfate, I-urobilinogen, 16-hydroxypalmitate, 3-hydroxydecanoate, 3-hydroxyoctanoate, 16a-hydroxy DHEA 3-sulfate, dehydroisoandrosterone sulfate (DHEA-S), 5-methylthioadenosine (MTA), valylglycine, cyclo (L-phe-L-pro), fucose, taurine, gamma-glutamylhistidine, 3-hydroxyisobutyrate, CE(24:1), PE(P-16:0/14:1), LPC(14:0), SM(18:1), PE(15:0/22:4), FFA(20:0), LPC(12:0), LCER(26:0), LPE(14:1), PI(16:0/16:0), LPE(20:4), DCER(20:0), LCER(14:0), PE(15:0/18:4), PI(18:0/16:1), PE(16:0/22:2), PE(P-14:1/18:1), PC(16:0/14:1), PE(18:0/17:0), PE(P-16:0/18:0), PE(P-18:0/16:1), PE(O-18:0/18:0), CER(26:0), PE(16:0/16:0), LPE(18:4), and PE(O-18:0/14:1). One or more additional biomarkers may optionally be selected from the group consisting of: TL16:1n7 (16:1n7, palmitoleic acid), TL16:0 (16:0, palmitic acid), taurocholate, glycocholate, taurochenodeoxycholate, glycochenodeoxycholate, glutamate, LPE(18:2), LPE(20:3), PE(14:0/14:1), PC(14:0/22:4), PC(15:0/16:1), PC(20:0/14:1), PC(17:0/22:6), PE(15:0/18:3), PE(17:0/20:2), PE(18:2/20:2), PE(18:2/20:3), PC(18:1/22:6), PC(18:1/22:5), PC(14:0/18:4), SM(16:0), CE(24:0), PC(14:0/20:2), PC(14:0/20:3), PC(18:1/18:4), SM(18:0), PC(14:0/18:2), and PC(14:0/16:1).

In another example, the levels of one or more biomarkers in Tables 2, 3, 4, 5, 7, 8, 10, 11, and/or 20 may be determined in the methods of distinguishing NASH from NAFLD in a subject. For example, one or more of the following biomarkers may be used alone or in combination to distinguish NASH from NAFLD: epiandrosterone sulfate, androsterone sulfate, I-urobilinogen, 16-hydroxypalmitate, fucose, taurine, 3-hydroxydecanoate, 3-hydroxyoctanoate, 16a-hydroxy DHEA 3-sulfate, dehydroisoandrosterone sulfate (DHEA-S), 5-methylthioadenosine (MTA), gamma-glutamylhistidine, valylglycine, 3-hydroxyisobutyrate, cyclo (L-phe-L-pro), 2-aminoadipate, 4-methyl-2-oxopentanoate, 2-hydroxybutyrate, prolylproline, tauro-beta-muricholate, CE(24:1), PE(P-16:0/14:1), LPC(14:0), SM(18:1), PE(15:0/22:4), FFA(20:0), LPC(12:0), LCER(26:0), LPE(14:1), PI(16:0/16:0), LPE(20:4), DCER(20:0), LCER(14:0), PE(15:0/18:4), PI(18:0/16:1), PE(16:0/22:2), PE(P-14:1/18:1), PC(16:0/14:1), PE(18:0/17:0), PE(P-16:0/18:0), PE(P-18:0/16:1), PE(O-18:0/18:0), CER(26:0), PE(16:0/16:0), LPE(18:4), and PE(O-18:0/14:1). One or more additional biomarkers may optionally be selected from the group consisting of: isoleucine, glutamate, alpha-ketoglutarate, TL16:1n7 (16:1n7, palmitoleic acid), TL16:0 (16:0, palmitic acid), taurocholate, glycocholate, taurochenodeoxycholate, glycochenodeoxycholate, glycine, serine, leucine, deoxycholate, 3-methyl-2-oxovalerate, valine, 3-methyl-2-oxobutyrate, lanosterol, LPE(18:2), LPE(20:3), PE(14:0/14:1), PC(14:0/22:4), PC(15:0/16:1), PC(20:0/14:1), PC(17:0/22:6), PE(15:0/18:3), PE(17:0/20:2), PE(18:2/20:2), PE(18:2/20:3), PC(18:1/22:6), PC(18:1/22:5), PC(14:0/18:4), SM(16:0), CE(24:0), PC(14:0/20:2), PC(14:0/20:3), PC(18:1/18:4), SM(18:0), PC(14:0/18:2), and PC(14:0/16:1).

In another example, the levels of one or more biomarkers in Tables 10, 11, 12, 14, 16, 18, and/or 20 may be determined in the methods of diagnosing or aiding in diagnosing whether a subject has fibrosis. For example, one or more of the following biomarkers may be used alone or in combination to diagnose or aid in diagnosing whether a subject has fibrosis: glutarate (pentanedioate), epiandrosterone sulfate, androsterone sulfate, I-urobilinogen, 16-hydroxypalmitate, fucose, taurine, 3-hydroxydecanoate, 3-hydroxyoctanoate, 16a-hydroxy DHEA 3-sulfate, dehydroisoandrosterone sulfate (DHEA-S), 2-aminoheptanoate, 5-methylthioadenosine (MTA), gamma-glutamylhistidine, valylglycine, cyclo(L-phe-L-pro), CER(14:0), DCER(14:0), LPE(12:0), DCER(18:0), PE(18:0/22:2), PE(P-18:0/18:3), LPC(17:0), LPC(22:0), CER(18:1), LCER(22:0), PE(16:0/20:1), CE(15:0), PE(16:0/22:4), PE(O-18:0/20:2), LPC(20:0), LPE(24:0), PC(12:0/14:1), PE(17:0/22:2), SM(18:1), CER(16:0), LCER(24:0), PE(O-18:0/20:3), CE(17:0), PE(P-16:0/18:3), PE(P-16:0/16:1), LPE(14:1), FFA(24:0), PE(O-16:0/18:4), FFA(15:0), SM(14:0), LPC(20:2), PE(P-14:1/18:1), SM(24:1), PI(18:0/20:2), LPC(15:0), PE(O-18:0/18:1), PI(18:1/20:3), PE(16:0/16:1), DAG(18:1/20:3)X-19561, X-18889, X-21471, X-11871, and X-12850. One or more additional biomarkers may optionally be selected from the group consisting of: taurocholate, glycocholate, taurochenodeoxycholate, glycochenodeoxycholate, glutamate, TL16:1n7 (16:1n7, palmitoleate), TL16:0 (16:0, palmitic acid), isoleucine, alpha-ketoglutarate, PE(18:2/20:2), PE(14:0/16:1), PE(14:0/14:1), PE(16:0/18:1), PE(18:1/18:1), PE(17:0/20:4), PE(14:0/20:5), PE(16:0/22:5), PE(18:2/20:3), PE(16:0/20:4), PE(14:0/18:2), PE(18:1/18:4), PE(15:0/22:6), PE(16:0/14:0), LPC(18:3), TAG55:7-FA20:3, TAG53:6-FA18:2, TAG55:7-FA20:4, TAG53:5-FA18:2, TAG53:7-FA18:3, TAG55:8-FA20:4, TAG53:5-FA18:1, TAG55:6-FA20:3, TAG57:9-FA22:6, TAG53:6-FA18:3, TAG55:6-FA18:1, TAG53:6-FA18:1, TAG53:4-FA18:1, TAG53:4-FA18:0, TAG51:4-FA16:0, TAG53:3-FA18:0, TAG51:3-FA16:0, TAG51:4-FA18:1, TAG56:5-FA20:4, TAG56:5-FA18:0, TAG56:4-FA20:4, PE(14:0/18:1), PC(14:0/18:4), PC(18:2/22:5), PC(20:0/22:5), SM(18:0), CE(18:0), PC(18:2/18:4), and PC(14:0/20:2).

In another example, the levels of one or more biomarkers in Tables 10, 11, 12, 14, 16, and/or 18 may be determined in the methods of determining the stage of fibrosis in a subject. For example, one or more of the following biomarkers may be used alone or in combination to diagnose or aid in diagnosing whether a subject has fibrosis: glutarate (pentanedioate), epiandrosterone sulfate, androsterone sulfate, I-urobilinogen, 16-hydroxypalmitate, fucose, taurine, 3-hydroxydecanoate, 3-hydroxyoctanoate, 16a-hydroxy DHEA 3-sulfate, dehydroisoandrosterone sulfate (DHEA-S), 2-aminoheptanoate, 5-methylthioadenosine (MTA), gamma-glutamylhistidine, valylglycine, and cyclo(L-phe-L-pro). One or more additional biomarkers may optionally be selected from the group consisting of: taurocholate, glycocholate, taurochenodeoxycholate, glycochenodeoxycholate, glutamate, TL16:1n7 (16:1n7, palmitoleate), TL16:0 (16:0, palmitic acid), isoleucine, and alpha-ketoglutarate.

After the level(s) of the one or more biomarkers in the sample are determined, the level(s) are compared to liver disease-positive and/or liver disease-negative reference levels to diagnose or aid in diagnosing whether the subject has liver disease. Levels of the one or more biomarkers in a sample matching the liver disease-positive reference levels (e.g., levels that are the same as the reference levels, substantially the same as the reference levels, above and/or below the minimum and/or maximum of the reference levels, and/or within the range of the reference levels) are indicative of a diagnosis of liver disease in the subject. Levels of the one or more biomarkers in a sample matching the liver disease-negative reference levels (e.g., levels that are the same as the reference levels, substantially the same as the reference levels, above and/or below the minimum and/or maximum of the reference levels, and/or within the range of the reference levels) are indicative of a diagnosis of no liver disease in the subject. In addition, levels of the one or more biomarkers that are differentially present (especially at a level that is statistically significant) in the sample as compared to liver disease-negative reference levels are indicative of a diagnosis of liver disease in the subject. Levels of the one or more biomarkers that are differentially present (especially at a level that is statistically significant) in the sample as compared to liver disease-positive reference levels are indicative of a diagnosis of no liver disease in the subject.

The level(s) of the one or more biomarkers may be compared to liver disease-positive and/or liver disease-negative reference levels using various techniques, including a simple comparison (e.g., a manual comparison) of the level(s) of the one or more biomarkers in the biological sample to liver disease-positive and/or liver disease-negative reference levels. The level(s) of the one or more biomarkers in the biological sample may also be compared to liver disease-positive and/or liver disease-negative reference levels using one or more statistical analyses (e.g., t-test, Welch's T-test, Wilcoxon's rank sum test, Random Forest, T-score, Z-score) or using a mathematical model (e.g., algorithm, statistical model, mixed effects model).

For example, a mathematical model comprising a single algorithm or multiple algorithms may be used to determine whether a subject has liver disease. A mathematical model may also be used to distinguish between types of liver disease (e.g., NASH and NAFLD) or between fibrosis stages. An exemplary mathematical model may use the measured levels of any number of biomarkers (for example, 2, 3, 5, 7, 9, etc.) from a subject to determine, using an algorithm or a series of algorithms based on mathematical relationships between the levels of the measured biomarkers, whether a subject has liver disease, whether liver disease is progressing or regressing in a subject, whether a subject has more advanced or less advanced liver disease, etc. In one example, the mathematical model is logistic regression modeling. In another example, the mathematical model is multiple logistic regression modeling.

The results of the method may be used along with other methods (or the results thereof) useful in the diagnosis of liver disease in a subject. For example, the results of the method may provide an indication of patients who warrant invasive follow-up testing (e.g., liver biopsy) to confirm the diagnosis of NAFLD, NASH, fibrosis or cirrhosis.

In one aspect, the biomarkers provided herein can be used to provide a physician with a Liver Disease Score (e.g., NASH Score, NAFLD Score, Fibrosis Score) indicating the existence and/or severity of liver disease in a subject. The Score is based upon clinically significantly changed reference level(s) for a biomarker and/or combination of biomarkers. The reference level can be derived from an algorithm. The Score can be used to place the subject in a severity range of liver disease from normal (i.e. no liver disease) to severe. The Score can be used in multiple ways: for example, disease progression, regression, or remission can be monitored by periodic determination and monitoring of the Score; response to therapeutic intervention can be determined by monitoring the Score; and drug efficacy can be evaluated using the Score.

Methods for determining a subject's liver disease Score may be performed using one or more of the liver disease biomarkers identified in Tables 2, 3, 4, 5, 7, 8, 10, 11, 12, 14, 16, 18, 19, and/or 20 in a biological sample. The method may comprise comparing the level(s) of the one or more liver disease biomarkers in the sample to liver disease reference levels of the one or more biomarkers in order to determine the subject's liver disease score. The method may employ any number of markers selected from those listed in Tables 2, 3, 4, 5, 7, 8, 10, 11, 12, 14, 16, 18, 19, and/or 20, including 1, 2, 3, 4, 5, 6, 7, 8, 9, 10, or more markers. Multiple biomarkers may be correlated with liver disease, by any method, including statistical methods such as regression analysis.

After the level(s) of the one or more biomarker(s) is determined, the level(s) may be compared to liver disease reference level(s) or reference curves of the one or more biomarker(s) to determine a rating for each of the one or more biomarker(s) in the sample. The rating(s) may be aggregated using any algorithm to create a score, for example, an liver disease score, for the subject. The algorithm may take into account any factors relating to liver disease including the number of biomarkers, the correlation of the biomarkers to liver disease, etc.

In an embodiment, a mathematical model or formula containing one or more biomarkers as variables is established using regression analysis, e.g., multiple linear regressions. By way of non-limiting example, the developed formulas may include the following:

A+B(Biomarker₁)+C(Biomarker₂)+D(Biomarker₃)+E(Biomarker₄)=RScor e

A+B*1n(Biomarker₁)+C*1n(Biomarker₂)+D*1n(Biomarker₃)+E*1n(Biomar ker₄)=1nRScore

wherein A, B, C, D, E are constant numbers; Biomarker₁, Biomarker₂, Biomarker₃, Biomarker₄ are the measured values of the analyte (Biomarker) and RScore is the measure of liver disease presence or absence or severity.

The formulas may include one or more biomarkers as variables, such as 1, 2, 3, 4, 5, 10, 15, 20 or more biomarkers.

### B. Methods of Monitoring Progression/Regression of Liver Disease

The identification of biomarkers for liver disease also allows for monitoring progression/regression of liver disease in a subject. A method of monitoring the progression/regression of liver disease in a subject comprises (1) analyzing a first biological sample from a subject to determine the level(s) of one or more biomarkers for liver disease selected from Tables 2, 3, 4, 5, 7, 8, 10, 11, 12, 14, 16, 18, 19, and/or 20, the first sample obtained from the subject at a first time point, (2) analyzing a second biological sample from a subject to determine the level(s) of the one or more biomarkers, the second sample obtained from the subject at a second time point, and (3) comparing the level(s) of one or more biomarkers in the first sample to the level(s) of the one or more biomarkers in the second sample in order to monitor the progression/regression of liver disease in the subject. The results of the method are indicative of the course of liver disease (i.e., progression or regression, if any change) in the subject.

The levels of one or more of the biomarkers of Tables 2, 3, 4, 5, 7, 8, 10, 11, 12, 14, 16, 18, 19, and/or 20 including a combination of all of the biomarkers in Tables 2, 3, 4, 5, 7, 8, 10, 11, 12, 14, 16, 18, 19, and/or 20 and combinations thereof or any fraction thereof, may be determined and used in methods of monitoring the progression/regression of liver disease in a subject. For example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 2, 3, 4, 5, 7, 8, 10, 11, 12, 14, 16, 18, 19, and/or 20 or any fraction thereof, may be determined and used in methods of monitoring the progression/regression of liver disease of a subject.

In one example, the levels of one or more biomarkers in Tables 2, 3, 4, 5, 7, 8, 10, and/or 11, may be determined in the methods of monitoring the progression/regression of NAFLD in a subject. For example, one or more of the following biomarkers may be used alone or in combination to monitor the progression/regression of NAFLD: epiandrosterone sulfate, androsterone sulfate, I-urobilinogen, 16-hydroxypalmitate, fucose, taurine, 3-hydroxydecanoate, 3-hydroxyoctanoate, 16a-hydroxy DHEA 3-sulfate, dehydroisoandrosterone sulfate (DHEA-S), 5-methylthioadenosine (MTA), gamma-glutamylhistidine, valylglycine, 3-hydroxyisobutyrate, cyclo (L-phe-L-pro), 2-aminoadipate, 4-methyl-2-oxopentanoate, 2-hydroxybutyrate, prolylproline, and tauro-beta-muricholate. One or more additional biomarkers may optionally be selected from the group consisting of: isoleucine, glutamate, alpha-ketoglutarate, TL16:1n7 (16:1n7, palmitoleic acid), TL16:0 (16:0, palmitic acid), taurocholate, glycocholate, taurochenodeoxycholate, glycochenodeoxycholate, glycine, serine, leucine, deoxycholate, 3-methyl-2-oxovalerate, valine, 3-methyl-2-oxobutyrate, and lanosterol.

In another example, the levels of one or more biomarkers in Tables 7, 8, 10, 11, and/or 20 and any combination thereof including a combination of all of the biomarkers may be determined in the methods of monitoring the progression/regression of NASH in a subject. For example, one or more of the following biomarkers may be used alone or in combination to diagnose or aid in diagnosing NASH: epiandrosterone sulfate, androsterone sulfate, I-urobilinogen, 16-hydroxypalmitate, 3-hydroxydecanoate, 3-hydroxyoctanoate, 16a-hydroxy DHEA 3-sulfate, dehydroisoandrosterone sulfate (DHEA-S), 5-methylthioadenosine (MTA), valylglycine, cyclo (L-phe-L-pro), fucose, taurine, gamma-glutamylhistidine, 3-hydroxyisobutyrate, CE(24:1), PE(P-16:0/14:1), LPC(14:0), SM(18:1), PE(15:0/22:4), FFA(20:0), LPC(12:0), LCER(26:0), LPE(14:1), PI(16:0/16:0), LPE(20:4), DCER(20:0), LCER(14:0), PE(15:0/18:4), PI(18:0/16:1), PE(16:0/22:2), PE(P-14:1/18:1), PC(16:0/14:1), PE(18:0/17:0), PE(P-16:0/18:0), PE(P-18:0/16:1), PE(O-18:0/18:0), CER(26:0), PE(16:0/16:0), LPE(18:4), and PE(O-18:0/14:1). One or more additional biomarkers may optionally be selected from the group consisting of: TL16:1n7 (16:1n7, palmitoleic acid), TL16:0 (16:0, palmitic acid), taurocholate, glycocholate, taurochenodeoxycholate, glycochenodeoxycholate, glutamate, LPE(18:2), LPE(20:3), PE(14:0/14:1), PC(14:0/22:4), PC(15:0/16:1), PC(20:0/14:1), PC(17:0/22:6), PE(15:0/18:3), PE(17:0/20:2), PE(18:2/20:2), PE(18:2/20:3), PC(18:1/22:6), PC(18:1/22:5), PC(14:0/18:4), SM(16:0), CE(24:0), PC(14:0/20:2), PC(14:0/20:3), PC(18:1/18:4), SM(18:0), PC(14:0/18:2), and PC(14:0/16:1).

In another example, the levels of one or more biomarkers in Tables 10, 11, 12, 14, 16, 18, and/or 20 may be determined in the methods of monitoring the progression/regression of fibrosis in a subject. For example, one or more of the following biomarkers may be used alone or in combination to monitor progression/regression of fibrosis in a subject: glutarate (pentanedioate), epiandrosterone sulfate, androsterone sulfate, I-urobilinogen, 16-hydroxypalmitate, fucose, taurine, 3-hydroxydecanoate, 3-hydroxyoctanoate, 16a-hydroxy DHEA 3-sulfate, dehydroisoandrosterone sulfate (DHEA-S), 2-aminoheptanoate, 5-methylthioadenosine (MTA), gamma-glutamylhistidine, valylglycine, cyclo(L-phe-L-pro), CER(14:0), DCER(14:0), LPE(12:0), DCER(18:0), PE(18:0/22:2), PE(P-18:0/18:3), LPC(17:0), LPC(22:0), CER(18:1), LCER(22:0), PE(16:0/20:1), CE(15:0), PE(16:0/22:4), PE(O-18:0/20:2), LPC(20:0), LPE(24:0), PC(12:0/14:1), PE(17:0/22:2), SM(18:1), CER(16:0), LCER(24:0), PE(O-18:0/20:3), CE(17:0), PE(P-16:0/18:3), PE(P-16:0/16:1), LPE(14:1), FFA(24:0), PE(O-16:0/18:4), FFA(15:0), SM(14:0), LPC(20:2), PE(P-14:1/18:1), SM(24:1), PI(18:0/20:2), LPC(15:0), PE(O-18:0/18:1), PI(18:1/20:3), PE(16:0/16:1), DAG(18:1/20:3)X-19561, X-18889, X-21471, X-11871, and X-12850. One or more additional biomarkers may optionally be selected from the group consisting of: taurocholate, glycocholate, taurochenodeoxycholate, glycochenodeoxycholate, glutamate, TL16:1n7 (16:1n7, palmitoleate), TL16:0 (16:0, palmitic acid), isoleucine, alpha-ketoglutarate, PE(18:2/20:2), PE(14:0/16:1), PE(14:0/14:1), PE(16:0/18:1), PE(18:1/18:1), PE(17:0/20:4), PE(14:0/20:5), PE(16:0/22:5), PE(18:2/20:3), PE(16:0/20:4), PE(14:0/18:2), PE(18:1/18:4), PE(15:0/22:6), PE(16:0/14:0), LPC(18:3), TAG55:7-FA20:3, TAG53:6-FA18:2, TAG55:7-FA20:4, TAG53:5-FA18:2, TAG53:7-FA18:3, TAG55:8-FA20:4, TAG53:5-FA18:1, TAG55:6-FA20:3, TAG57:9-FA22:6, TAG53:6-FA18:3, TAG55:6-FA18:1, TAG53:6-FA18:1, TAG53:4-FA18:1, TAG53:4-FA18:0, TAG51:4-FA16:0, TAG53:3-FA18:0, TAG51:3-FA16:0, TAG51:4-FA18:1, TAG56:5-FA20:4, TAG56:5-FA18:0, TAG56:4-FA20:4, PE(14:0/18:1), PC(14:0/18:4), PC(18:2/22:5), PC(20:0/22:5), SM(18:0), CE(18:0), PC(18:2/18:4), and PC(14:0/20:2).

The change (if any) in the level(s) of the one or more biomarkers over time may be indicative of progression or regression of liver disease in the subject. In order to characterize the course of liver disease in the subject, the level(s) of the one or more biomarkers in the first sample, the level(s) of the one or more biomarkers in the second sample, and/or the results of the comparison of the levels of the biomarkers in the first and second samples may be compared to liver disease-positive and liver disease-negative reference levels. If the comparisons indicate that the level(s) of the one or more biomarkers are increasing or decreasing over time (e.g., in the second sample as compared to the first sample) to become more similar to the liver disease-positive reference levels (or less similar to the liver disease-negative reference levels), then the results are indicative of liver disease progression. If the comparisons indicate that the level(s) of the one or more biomarkers are increasing or decreasing over time to become more similar to the liver disease-negative reference levels (or less similar to the liver disease-positive reference levels), then the results are indicative of liver disease regression.

In one embodiment, the assessment may be based on a liver disease Score (e.g., NASH Score, NAFLD Score, Fibrosis Score) which is indicative of liver disease in the subject and which can be monitored over time. By comparing the liver disease Score from a first time point sample to the liver disease Score from at least a second time point sample the progression or regression of liver disease can be determined. Such a method of monitoring the progression/regression of liver disease in a subject comprises (1) analyzing a first biological sample from a subject to determine a liver disease score for the first sample obtained from the subject at a first time point, (2) analyzing a second biological sample from a subject to determine a second liver disease score, the second sample obtained from the subject at a second time point, and (3) comparing the liver disease score in the first sample to the liver disease score in the second sample in order to monitor the progression/regression of liver disease in the subject.

The biomarkers and algorithms described herein may guide or assist a physician in deciding a treatment path, for example, whether to implement procedures such as surgical procedures (e.g., full or partial nephrectomy), treat with drug therapy, or employ a watchful waiting approach.

As with the other methods described herein, the comparisons made in the methods of monitoring progression/regression of liver disease in a subject may be carried out using various techniques, including simple comparisons, one or more statistical analyses, mathematical models (algorithms) and combinations thereof.

The results of the method may be used along with other methods (or the results thereof) useful in the clinical monitoring of progression/regression of liver disease in a subject.

As described above in connection with methods of diagnosing (or aiding in the diagnosis of) liver disease, any suitable method may be used to analyze the biological samples in order to determine the level(s) of the one or more biomarkers in the samples. In addition, the level(s) one or more biomarkers, including a combination of all of the biomarkers in Tables 2, 3, 4, 5, 7, 8, 10, 11, 12, 14, 16, 18, 19, and/or 20 or any fraction thereof, may be determined and used in methods of monitoring progression/regression of liver disease in a subject.

Such methods could be conducted to monitor the course of liver disease in subjects having liver disease or could be used in subjects not having liver disease (e.g., subjects suspected of being predisposed to developing liver disease) in order to monitor levels of predisposition to liver disease.

C. Methods of Staging Liver Fibrosis

The identification of biomarkers for liver disease also allows for the determination of the liver fibrosis stage of a subject. A method of determining the stage of fibrosis comprises (1) analyzing a biological sample from a subject to determine the level(s) of one or more biomarkers listed in Tables 10 11, 12, 14, 16, and/or 18 in the sample and (2) comparing the level(s) of the one or more biomarkers in the sample to high stage fibrosis and/or low stage fibrosis reference levels of the one or more biomarkers in order to determine the stage of the subject's liver fibrosis. The results of the method may be used along with other methods (or the results thereof) useful in the clinical determination of the stage of a subject's liver disease. For example, the results of the method may provide an indication of patients who warrant invasive follow-up testing (e.g., liver biopsy) when a diagnosis is NAFLD or NASH is suspected based on the stage of liver fibrosis.

As described above in connection with methods of diagnosing (or aiding in the diagnosis of) liver disease, any suitable method may be used to analyze the biological sample in order to determine the level(s) of the one or more biomarkers in the sample.

The levels of one or more biomarkers listed in Tables 10, 11, 12, 14, 16, and/or 18 and combinations thereof may be determined in the methods of determining the stage of a subject's liver fibrosis. For example, the level(s) of one biomarker, two or more biomarkers, three or more biomarkers, four or more biomarkers, five or more biomarkers, six or more biomarkers, seven or more biomarkers, eight or more biomarkers, nine or more biomarkers, ten or more biomarkers, etc., including a combination of all of the biomarkers in Tables 10, 11, 12, 14, 16, and/or 18 or any fraction thereof, may be determined and used in methods of determining the stage of liver disease of a subject. For example, one or more of the following biomarkers may be used alone or in combination to diagnose or aid in diagnosing whether a subject has fibrosis: glutarate (pentanedioate), epiandrosterone sulfate, androsterone sulfate, I-urobilinogen, 16-hydroxypalmitate, fucose, taurine, 3-hydroxydecanoate, 3-hydroxyoctanoate, 16a-hydroxy DHEA 3-sulfate, dehydroisoandrosterone sulfate (DHEA-S), 2-aminoheptanoate, 5-methylthioadenosine (MTA), gamma-glutamylhistidine, valylglycine, and cyclo(L-phe-L-pro). One or more additional biomarkers may optionally be selected from the group consisting of: taurocholate, glycocholate, taurochenodeoxycholate, glycochenodeoxycholate, glutamate, TL16:1n7 (16:1n7, palmitoleate), TL16:0 (16:0, palmitic acid), isoleucine, and alpha-ketoglutarate.

After the level(s) of the one or more biomarkers in a sample are determined, the level(s) are compared to low stage liver fibrosis and/or high stage liver fibrosis reference levels in order to predict the stage of liver fibrosis of a subject. Levels of the one or more biomarkers in a sample matching the high stage liver fibrosis reference levels (e.g., levels that are the same as the reference levels, substantially the same as the reference levels, above and/or below the minimum and/or maximum of the reference levels, and/or within the range of the reference levels) are indicative of the subject having high stage liver fibrosis. Levels of the one or more biomarkers in a sample matching the low stage liver fibrosis reference levels (e.g., levels that are the same as the reference levels, substantially the same as the reference levels, above and/or below the minimum and/or maximum of the reference levels, and/or within the range of the reference levels) are indicative of the subject having low stage liver fibrosis. In addition, levels of the one or more biomarkers that are differentially present (especially at a level that is statistically significant) in the sample as compared to low stage liver fibrosis reference levels are indicative of the subject not having low stage liver fibrosis. Levels of the one or more biomarkers that are differentially present (especially at a level that is statistically significant) in the sample as compared to high stage liver fibrosis reference levels are indicative of the subject not having high stage liver fibrosis.

Studies were carried out to identify a set of biomarkers that can be used to determine the liver fibrosis stage of a subject. In another embodiment, the biomarkers provided herein can be used to provide a physician with a Fibrosis Score indicating the stage of liver fibrosis in a subject. The score is based upon clinically significantly changed reference level(s) for a biomarker and/or combination of biomarkers. The reference level can be derived from an algorithm. The Fibrosis Score can be used to determine the stage of liver fibrosis in a subject from normal (i.e. no liver fibrosis, Stage 0) to high stage liver fibrosis (i.e., Stage 3-4).

As with the methods described above, the level(s) of the one or more biomarkers may be compared to high stage liver fibrosis and/or low stage liver fibrosis reference levels using various techniques, including a simple comparison, one or more statistical analyses, and combinations thereof.

D. Methods of Distinguishing Less Severe Liver Disease from More Severe Liver Disease

The identification of biomarkers for liver disease also allows for the identification of biomarkers for distinguishing less severe liver disease from more severe liver disease. A method of distinguishing less severe liver disease from more severe liver disease in a subject having liver disease comprises (1) analyzing a biological sample from a subject to determine the level(s) of one or more biomarkers listed in Tables 2, 3, 4, 5, 7, 8, 10, 11, 12, 14, 16, 18, 19, and/or 20 in the sample and (2) comparing the level(s) of the one or more biomarkers in the sample to less severe liver disease and/or more severe liver disease reference levels of the one or more biomarkers in order to determine the severity of the subject's liver disease. The results of the method may be used along with other methods (or the results thereof) useful in the clinical determination of the severity of a subject's liver disease.

As described above in connection with methods of diagnosing (or aiding in the diagnosis of) liver disease, any suitable method may be used to analyze the biological sample in order to determine the level(s) of the one or more biomarkers in the sample.

In one example, the levels of one or more biomarkers listed in Tables 2, 3, 4, 5, 7, 8, 10, 11, 12, 14, 16, 18, 19, and/or 20, and any combination thereof including a combination of all of the biomarkers may be determined in the methods of determining the severity of a subject's liver disease. In one example, NAFLD is liver disease of low severity, borderline NASH is liver disease of moderate severity, and NASH is liver disease of high severity. In another example, Stage 0 liver fibrosis is liver disease of low severity, Stage 1-2 liver fibrosis is liver disease of moderate severity, and Stage 3-4 fibrosis is liver disease of high severity. In another example, NASH is a liver disease of high severity, and non-NASH is a liver disease of low severity. In another example, fibrosis is a liver disease of high severity, and non-fibrosis is a liver disease of low severity. In another example, NAFLD is a liver disease of higher severity than non-NAFLD.

After the level(s) of the one or more biomarkers in the sample are determined, the level(s) are compared to less severe liver disease and/or more severe liver disease reference levels in order to determine the aggressiveness of liver disease of a subject. Levels of the one or more biomarkers in a sample matching the more severe liver disease reference levels (e.g., levels that are the same as the reference levels, substantially the same as the reference levels, above and/or below the minimum and/or maximum of the reference levels, and/or within the range of the reference levels) are indicative of the subject having more severe liver disease. Levels of the one or more biomarkers in a sample matching the less severe liver disease reference levels (e.g., levels that are the same as the reference levels, substantially the same as the reference levels, above and/or below the minimum and/or maximum of the reference levels, and/or within the range of the reference levels) are indicative of the subject having less severe liver disease. In addition, levels of the one or more biomarkers that are differentially present (especially at a level that is statistically significant) in the sample as compared to less severe liver disease reference levels are indicative of the subject not having less severe liver disease. Levels of the one or more biomarkers that are differentially present (especially at a level that is statistically significant) in the sample as compared to more severe liver disease reference levels are indicative of the subject not having more severe liver disease.

Studies were carried out to identify a set of biomarkers that can be used to distinguish less severe liver disease from more severe liver disease. In another embodiment, the biomarkers provided herein can be used to provide a physician with a liver disease Score indicating the severity of liver disease in a subject. The score is based upon clinically significantly changed reference level(s) for a biomarker and/or combination of biomarkers. The reference level can be derived from an algorithm. The liver disease Score can be used to determine the severity of liver disease in a subject from normal (i.e. no liver disease) to more severe liver disease.

As with the methods described above, the level(s) of the one or more biomarkers may be compared to more severe liver disease and/or less severe liver disease reference levels using various techniques, including a simple comparison, one or more statistical analyses, and combinations thereof.

As with the methods of diagnosing (or aiding in diagnosing) whether a subject has liver disease, the methods of determining the severity of liver disease of a subject may further comprise analyzing the biological sample to determine the level(s) of one or more non-biomarker compounds.

### III. Other Methods

Other methods of using the biomarkers discussed herein are also contemplated. For example, the methods described in U.S. Pat. No. 7,005,255, U.S. Pat. No. 7,329,489, U.S. Pat. No. 7,553,616, U.S. Pat. No. 7,550,260, U.S. Pat. No. 7,550,258, U.S. Pat. No. 7,635,556, U.S. patent application Ser. No. 11/728,826, U.S. patent application Ser. No. 12/463,690 and U.S. patent application Ser. No. 12/182,828 may be conducted using a small molecule profile comprising one or more of the biomarkers disclosed herein.

In any of the methods listed herein, the biomarkers that are used may be selected from those biomarkers in Tables 2, 3, 4, 5, 7, 8, 10, 11, 12, 14, 16, and/or 18 having p-values of less than 0.05. The biomarkers that are used in any of the methods described herein may also be selected from those biomarkers in Tables 2, 3, 4, 5, 7, 8, 10, 11, 12, 14, 16, and/or 18 that are decreased in liver disease (as compared to the control) or that are decreased in high stage fibrosis (as compared to control or low stage fibrosis) or that are decreased in more severe (as compared to control or less severe) by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by 100% (i.e., absent); and/or those biomarkers in Tables 2, 3, 4, 5, 7, 8, 10, 11, 12, 14, 16, and/or 18 that are increased in the liver disease (as compared to the control) or that are increased high stage fibrosis (as compared to control or low stage fibrosis) or that are increased in more severe (as compared to control or less severe) by at least 5%, by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%, by at least 110%, by at least 120%, by at least 130%, by at least 140%, by at least 150%, or more.

### EXAMPLES

The invention will be further explained by the following illustrative examples that are intended to be non-limiting.

### I. General Methods

### A. Sample Preparation.

Samples were prepared using the automated MicroLab STAR® system from Hamilton Company. Recovery standards were added prior to the first step in the extraction process for QC purposes. Sample preparation was conducted using a methanol extraction to remove the protein fraction while allowing maximum recovery of small molecules. The resulting extract was divided into five fractions: one for analysis by UPLC-MS/MS with positive ion mode electrospray ionization, one for analysis by UPLC-MS/MS with negative ion mode electrospray ionization, one for LC polar platform, one for analysis by GC-MS, and one sample was reserved for backup. Samples were placed briefly on a TurboVap® (Zymark) under nitrogen to remove the organic solvent. For LC, the samples were stored under nitrogen overnight. For GC, the samples were dried under vacuum overnight. Samples were then prepared for the appropriate instrument, either LC/MS or GC/MS.

### B. Ultrahigh Performance Liquid Chromatography-Tandem Mass Spectroscopy (UPLC-MS/MS).

LC/MS analysis used a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The sample extract was dried then reconstituted in acidic or basic LC-compatible solvents, each of which contained 8 or more injection standards at fixed concentrations to ensure injection and chromatographic consistency. One aliquot was analyzed using acidic positive ion optimized conditions and the other using basic negative ion optimized conditions in two independent injections using separate dedicated columns (Waters UPLC BEH C18-2.1×100 mm, 1.7 μm). Extracts reconstituted in acidic conditions were gradient eluted from a C18 column using water and methanol containing 0.1% formic acid. The basic extracts were similarly eluted from C18 using methanol and water containing with 6.5mM Ammonium Bicarbonate. The third aliquot was analyzed via negative ionization following elution from a HILIC column (Waters UPLC BEH Amide 2.1×150 mm, 1.7 μm) using a gradient consisting of water and acetonitrile with 10mM Ammonium Formate. The MS analysis alternated between MS and data-dependent MS2 scans using dynamic exclusion, and the scan range was from 80-1000 m/z.

### C. Gas Chromatography/Mass Spectrometry (GC/MS).

For GC/MS analysis, samples were re-dried under vacuum desiccation for a minimum of 24 hours prior to being derivatized under dried nitrogen using bistrimethyl-silyl-trifluoroacetamide (BSTFA). The GC column was a 20 m×0.18 mm ID, with 5% phenyl; 95% dimethylsilicone phase. The temperature ramp was from 60° to 340° C. in an 18 minute period. Samples were analyzed on a Thermo-Finnigan Trace DSQ fast-scanning single-quadrupole mass spectrometer using electron impact ionization at unit mass resolution. The instrument was tuned and calibrated for mass resolution and mass accuracy on a daily basis.

### D. Lipid Analysis.

### GC-FID

In some examples, lipids were extracted in the presence of authentic internal standards by the method of Folch et al. (J Biol Chem 226:497-509) using chloroform:methanol (2:1 v/v). Lipids were transesterified in 1% sulfuric acid in methanol in a sealed vial under a nitrogen atmosphere at 100° C. for 45 minutes. The resulting fatty acid methyl esters were extracted from the mixture with hexane containing 0.05% butylated hydroxytoluene and prepared for GC by sealing the hexane extracts under nitrogen. Fatty acid methyl esters were separated and quantified by capillary GC (Agilent Technologies 6890 Series GC) equipped with a 30 m DB 88 capillary column (Agilent Technologies) and a flame ionization detector. The absolute concentration of each lipid is determined by comparing the peak area to that of the internal standard.

### TRUEMASS Complex Lipid Panel

In some examples, lipids were extracted from samples in methanol:dichloromethane in the presence of internal standards. The extracts were concentrated under nitrogen and reconstituted in 0.25 mL of 10 MM ammonium acetate dichloromethane:methanol (50:50). The extracts were transferred to inserts and placed in vials for infusion-MS analysis, performed on a Shimazdu LC with nano PEEk tubing and a Sciex Selexlon-5500 QTRAP. The samples were analyzed via both positiove and negative mode electorspray. The 5500 QTRAP scan is performed in MRM mode with the total of more than 1,100 MRMs. Individual lipid species were quantified by taking the peak area ratios of target compounds and their assigned internal standards, then multiplying by the concentration of internal standard added to the sample. Lipid class concentrations were calculated from the sum of all molecular species within a class, and fatty acid compositions were determined by calculating the proportion of each class comprised by individual fatty acids.

### E. Data Processing and Analysis.

For each biological matrix data set on each instrument (except for GC-FID), relative standard deviations (RSDs) of peak area were calculated for each internal standard to confirm extraction efficiency, instrument performance, column integrity, chromatography, and mass calibration. Several of these internal standards serve as retention index (RI) markers and were checked for retention time and alignment. Modified versions of the software accompanying the UPLC-MS and GC-MS systems were used for peak detection and integration. The output from this processing generated a list of m/z ratios, retention times and area under the curve values. Software specified criteria for peak detection including thresholds for signal to noise ratio, height and width.

The biological data sets, including QC samples, were chromatographically aligned based on a retention index that utilizes internal standards assigned a fixed RI value. The RI of the experimental peak is determined by assuming a linear fit between flanking RI markers whose values do not change. The benefit of the RI is that it corrects for retention time drifts that are caused by systematic errors such as sample pH and column age. Each compound's RI was designated based on the elution relationship with its two lateral retention markers. Using an in-house software package, integrated, aligned peaks were matched against an in-house library (a chemical library) of authentic standards and routinely detected unknown compounds, which is specific to the positive, negative or GC-MS data collection method employed. Matches were based on retention index values within 150 RI units of the prospective identification and experimental precursor mass match to the library authentic standard within 0.4 m/z for the LTQ and DSQ data. The experimental MS/MS was compared to the library spectra for the authentic standard and assigned forward and reverse scores. A perfect forward score would indicate that all ions in the experimental spectra were found in the library for the authentic standard at the correct ratios and a perfect reverse score would indicate that all authentic standard library ions were present in the experimental spectra and at correct ratios. The forward and reverse scores were compared and a MS/MS fragmentation spectral score was given for the proposed match. All matches were then manually reviewed by an analyst that approved or rejected each call based on the criteria above. However, manual review by an analyst is not required. In some embodiments the matching process is completely automated.

Further details regarding a chemical library, a method for matching integrated aligned peaks for identification of named compounds and routinely detected unknown compounds, and computer-readable code for identifying small molecules in a sample may be found in U.S. Pat. No. 7,561,975, which is incorporated by reference herein in its entirety.

### F. Quality Control.

From the biological samples, aliquots of each of the individual samples were combined to make technical replicates, which were extracted as described above. Extracts of this pooled sample were injected six times for each data set on each instrument to assess process variability. As an additional quality control, five water aliquots were also extracted as part of the sample set on each instrument to serve as process blanks for artifact identification. All QC samples included the instrument internal standards to assess extraction efficiency, and instrument performance and to serve as retention index markers for ion identification. The standards were isotopically labeled or otherwise exogenous molecules chosen so as not to obstruct detection of intrinsic ions.

### G. Statistical Analysis.

Missing values, if any, were imputed with the observed minimum for that particular compound. A mixed-effects model was used to analyze differences between the NAFLD and non-NAFLD groups, and correlations between metabolites and clinical parameters were also assessed with a mixed-effects model. Statistical analyses were performed on natural log-transformed data. Random forest (RF) analysis was carried out to determine the ability of the global biochemical profile to separate the NAFLD and non-NAFLD groups and to separate groups based on fibrosis stage. Logistic regression and area under the curve (AUC) were used to assess the performance of individual metabolite biomarkers and several clinical parameters for distinguishing NAFLD from non-NAFLD and for distinguishing fibrosis stage. Logistic regression with Chi-square analysis and AUC were used to assess the performance of individual metabolite biomarkers for distinguishing fibrosis from no fibrosis and NASH from no NASH. Multiple logistic regression modeling was performed to analyze the performance of combinations of multiple biomarkers (biomarker panels).

### Example 1

### Metabolite Biomarkers of NAFLD in Human Serum

Serum samples from 36 subjects with NAFLD (as defined by >5% steatosis by MRI imaging) and 118 subjects without NAFLD were analyzed using four global metabolic profiling mass spectrometry platforms, as well as the GC-FID analysis for fatty acids, cholesterol metabolism lipids, and Vitamin E. A total of 770 named metabolites were detected in the patient samples. Clinical parameters including Age, Gender, Race, Ethnicity, Height/Weight/Body mass index (BMI), Smoking history, Diabetes history, Glucose, Albumin, Bilirubin, Aspartate aminotransferase (AST), Alanine aminotransferase (ALT), Alkaline phosphatase, Total cholesterol, High-density lipoprotein cholesterol (HDL), Low-density lipoprotein cholesterol (LDL), Triglycerides, Ferritin, Gamma-glutamyl transferase (GGT), HBA1c, White blood cell (WBC) count, Hemoglobin (HGB), Hematocrit (HCT), Platelet count, Prothrombin time, International normalized ratio (INR), Insulin, and Hepatic imaging parameters including MRI Proton Density Fat Fraction (MRI PDFF) and MRE (Elastography) were provided for the subjects. Data from MRI PDFF were used in the clinical determination of NAFLD or non-NAFLD.

Random forest (RF) analysis was carried out to determine the ability of the global biochemical profile to separate the NAFLD and non-NAFLD groups. RF is an unbiased and supervised classification technique based on a large number of decision trees. Using the groupings of NAFLD and non-NAFLD, RF classification analysis based on the serum metabolic profile of the entire study cohort (n=154) differentiated the two groups with 83.1% accuracy. Using all named metabolites, 83.9% (99 of 118) non-NAFLD and 80.6% (29 of 36) NAFLD subjects were correctly classified for an overall predictive accuracy of 83.1%.

Logistic regression and area under the curve (AUC) were used to assess the performance of several of the clinical parameters for distinguishing NAFLD from non-NAFLD. The results are shown in Table 1. Since MRI PDFF was used to diagnose NAFLD in this patient cohort, the AUC for that parameter is 1.000.

TABLE 1
AUC values for select clinical parameters

| Clinical Paramenter | AUC |
|---|---|
| Age | 0.643 |
| ALT | 0.734 |
| AST | 0.627 |
| BMI | 0.822 |
| Gender | 0.612 |
| Glucose | 0.715 |
| Insulin | 0.827 |
| MRE | 0.821 |
| MRI PDFF | 1.000 |

Logistic regression models and area under the curve (AUC) were used to assess how well individual metabolites discriminated the NAFLD and non-NAFLD groups. Logistic regression analysis was performed using the measured values obtained for all 770 named metabolites that were detected in the sample. The metabolites with an AUC of >0.700 for distinguishing NAFLD from non-NAFLD patient samples are presented in Table 2.

TABLE 2
AUC of individual metabolites for distinguishing NAFLD from non-NAFLD

| Metabolite | AUC | Metabolite | AUC |
|---|---|---|---|
| 5-methylthioadenosine (MTA) | 0.7606 | allantoin | 0.7403 |
| 3-hydroxyisobutyrate | 0.7232 | gamma-glutamylglutamate | 0.7388 |
| cyclo (L-phe-L-pro) | 0.7959 | 3-hydroxy-3-methylglutarate | 0.7378 |
| 2-aminoadipate | 0.8145 | S-adenosylhomocysteine (SAH) | 0.7368 |
| isoleucine | 0.7385 | 6-oxopiperidine-2-carboxylic | 0.7361 |
| | | acid | |
| glutamate | 0.7884 | erythronate | 0.7355 |
| alpha-ketoglutarate | 0.7394 | TL18:0 (stearic acid) | 0.7354 |
| TL16:1n7 (palmitoleic acid) | 0.7526 | N-acetyltyrosine | 0.7349 |
| TL16:0 (palmitic acid) | 0.7780 | N-formylmethionine | 0.7349 |
| succinylcarnitine | 0.7979 | urate | 0.7349 |
| N-acetylleucine | 0.7959 | TLTL (Total Total Lipid) | 0.7331 |
| N6- | 0.7943 | N2-methylguanosine | 0.7301 |
| carbamoylthreonyladenosine | | | |
| gamma-glutamylisoleucine | 0.7940 | C-glycosyltryptophan | 0.7275 |
| N-acetylphenylalanine | 0.7933 | arabinose | 0.7255 |
| N1-methyladenosine | 0.7903 | 3-methyl-2-oxobutyrate | 0.7243 |
| TL18:1n9 (oleic acid) | 0.7860 | N-acetylisoleucine | 0.7239 |
| gamma-glutamylvaline | 0.7839 | kynurenine | 0.7236 |
| gamma-glutamylleucine | 0.7805 | TL18:1n7 (avaccenic acid) | 0.7232 |
| N2,N2-dimethylguanosine | 0.7801 | glycerophosphorylcholine (GPC) | 0.7223 |
| 3-(4-hydroxyphenyl)lactate | 0.7779 | 3-methyl-2-oxovalerate | 0.7223 |
| N-acetylvaline | 0.7713 | lactate | 0.7186 |
| gamma-glutamyltyrosine | 0.7686 | 2-methylmalonyl carnitine | 0.7185 |
| xanthosine | 0.7677 | 7alpha-hydroxycholesterol | 0.7175 |
| N1-methylguanosine | 0.7669 | 1,7-dimethylurate | 0.7170 |
| TL14:0 (myristic acid) | 0.7648 | caffeine | 0.7150 |
| pseudouridine | 0.7571 | prolylproline | 0.7149 |
| cyclo(leu-pro) | 0.7562 | propionylcarnitine | 0.7145 |
| 4-hydroxyphenylpyruvate | 0.7549 | N4-acetylcytidine | 0.7142 |
| N-acetylalanine | 0.7527 | hydantoin-5-propionic acid | 0.7133 |
| TL14:1n5 (myristoleic acid) | 0.7514 | 7-methylguanine | 0.7129 |
| N-acetylserine | 0.7500 | indolelactate | 0.7121 |
| allo-isoleucine | 0.7498 | TL20:3n6 (di-homo-g-linoleic | 0.7119 |
| | | acid) | |
| glucose | 0.7495 | cystine | 0.7112 |
| maleate (cis-Butenedioate) | 0.7485 | valine | 0.7105 |
| 1,3,7-trimethylurate | 0.7479 | 3-hydroxy-2-ethylpropionate | 0.7063 |
| gamma-glutamylphenylalanine | 0.7475 | glucuronate | 0.7055 |
| alanine | 0.7459 | mannose | 0.7047 |
| glycine | 0.7456 | orotidine | 0.7039 |
| N6-acetyllysine | 0.7455 | gulonic acid | 0.7032 |
| pregnanediol-3-glucuronide | 0.7434 | 2-methylbutyrylcarnitine (C5) | 0.7032 |
| erythritol | 0.7432 | phenylcarnitine | 0.7023 |
| N-acetyltryptophan | 0.7419 | 4-methyl-2-oxopentanoate | 0.7023 |
| N6-succinyladenosine | 0.7411 | leucine | 0.7022 |

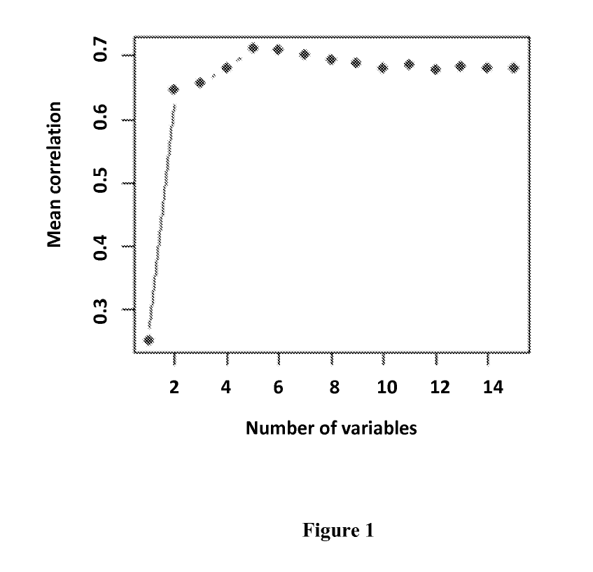

Multiple logistic regression modeling was performed to analyze the performance of various combinations of biomarkers (“biomarker panels”). The leave one out cross validation method was used to determine a number of variables (e.g., metabolite biomarkers) to include in the model. In this method one sample is removed from the data set, the model is fit on the remaining data and then the fitted model is used to predict the sample that was left out. The method provides an estimate of future performance. Here the clinical parameter MRI Proton Density Fat Fraction (MRI PDFF) was used to assess the change in the correlation as more variables are added to the model. As the number of compounds increases, the mean R² value for the correlation increases until an optimal number is reached, indicating that variable selection is more or less stable. In this analysis models with at least 2 variables increased the correlation and the correlation peaked at five variables. FIG. 1 shows the graph of the results of the correlation analysis. The number of markers is plotted on the X-axis and the mean correlation with MRI PDFF is plotted on the y-axis. Based on this analysis, the performance of 4-variable and 5-variable models were assessed. Models using 4 and 5 variables are exemplified below. It is apparent from the results illustrated in FIG. 1 that models may be comprised of more than 5 variables.

In one example, multiple logistic regression modeling with 4 and 5 variable models was performed using the measured values obtained for 13 metabolite biomarkers for distinguishing patients with NAFLD from individuals without NAFLD. These biomarkers included glycine, serine, leucine, 4-methyl-2-oxopentanoate, 3-methyl-2-oxovalerate, valine, 3-methyl-2-oxobutyrate, 2-hydroxybutyrate, 5-methylthioadenosine, prolylproline, lanosterol, tauro-beta-muricholate, and deoxycholate. There were 715 4-variable models generated using the listed 13 metabolites. The AUC was >0.800 for 204 of these models. There were 1287 5-variable models generated using the 13 listed metabolites. The AUC was >0.800 for 493 of these models. Table 3 shows the 30 4-variable models having the highest AUC. Table 4 shows the top 30 5-variable models. Table 5 shows the 13 metabolites used in the 4- and 5-variable models and the prevalence (in percentage) of the given metabolite in the models with AUC >0.800. For example, 5-methylthioadenosine (MTA) was identified in 92.2% of all 204 4-variable models with an AUC>0.800 and in 93.5% of all 493 5-variable models with an AUC>0.800.

TABLE 3
Top 30 4-variable models

| MODEL | AUC |
|---|---|
| 3-methyl-2-oxobutyrate + 5-methylthioadenosine (MTA) + prolylproline + | 0.885829 |
| lanosterol | |
| 3-methyl-2-oxovalerate + 5-methylthioadenosine (MTA) + prolylproline + | 0.884887 |
| lanosterol | |
| 4-methyl-2-oxopentanoate + 5-methylthioadenosine (MTA) + prolylproline + | 0.884181 |
| lanosterol | |
| glycine + 3-methyl-2-oxovalerate + 5-methylthioadenosine (MTA) + lanosterol | 0.882062 |
| glycine + 5-methylthioadenosine (MTA) + prolylproline + lanosterol | 0.879473 |
| glycine + 4-methyl-2-oxopentanoate + 5-methylthioadenosine (MTA) + | 0.876883 |
| lanosterol | |
| 3-methyl-2-oxovalerate + 5-methylthioadenosine (MTA) + lanosterol + tauro- | 0.876648 |
| beta-muricholate | |
| serine + 3-methyl-2-oxobutyrate + 5-methylthioadenosine (MTA) + | 0.874529 |
| prolylproline | |
| serine + 4-methyl-2-oxopentanoate + 5-methylthioadenosine (MTA) + lanosterol | 0.874294 |
| glycine + 3-methyl-2-oxobutyrate + 5-methylthioadenosine (MTA) + | 0.873823 |
| prolylproline | |
| serine + 3-methyl-2-oxobutyrate + 5-methylthioadenosine (MTA) + lanosterol | 0.872411 |
| serine + 3-methyl-2-oxovalerate + 5-methylthioadenosine (MTA) + prolylproline | 0.871704 |
| 3-methyl-2-oxobutyrate + 5-methylthioadenosine (MTA) + prolylproline + | 0.871704 |
| tauro-beta-muricholate | |
| glycine + 3-methyl-2-oxobutyrate + 5-methylthioadenosine (MTA) + lanosterol | 0.871469 |
| serine + 3-methyl-2-oxovalerate + 5-methylthioadenosine (MTA) + lanosterol | 0.86935 |
| 3-methyl-2-oxovalerate + 3-methyl-2-oxobutyrate + 5-methylthioadenosine | 0.86935 |
| (MTA) + lanosterol | |
| 4-methyl-2-oxopentanoate + 5-methylthioadenosine (MTA) + lanosterol + | 0.869115 |
| deoxycholate | |
| 3-methyl-2-oxovalerate + 5-methylthioadenosine (MTA) + lanosterol + | 0.869115 |
| deoxycholate | |
| glycine + 4-methyl-2-oxopentanoate + 5-methylthioadenosine (MTA) + | 0.868173 |
| prolylproline | |
| 3-methyl-2-oxobutyrate + 5-methylthioadenosine (MTA) + lanosterol + tauro- | 0.867938 |
| beta-muricholate | |
| 4-methyl-2-oxopentanoate + 5-methylthioadenosine (MTA) + lanosterol + tauro- | 0.867232 |
| beta-muricholate | |
| glycine + leucine + 5-methylthioadenosine (MTA) + lanosterol | 0.866996 |
| glycine + 3-methyl-2-oxovalerate + 5-methylthioadenosine (MTA) + | 0.866996 |
| prolylproline | |
| 4-methyl-2-oxopentanoate + 3-methyl-2-oxobutyrate + 5-methylthioadenosine | 0.866996 |
| (MTA) + lanosterol | |
| leucine + 3-methyl-2-oxobutyrate + 5-methylthioadenosine (MTA) + lanosterol | 0.866525 |
| 4-methyl-2-oxopentanoate + 3-methyl-2-oxovalerate + 5-methylthioadenosine | 0.866525 |
| (MTA) + lanosterol | |
| 5-methylthioadenosine (MTA) + prolylproline + lanosterol + tauro-beta- | 0.86629 |
| muricholate | |
| leucine + 4-methyl-2-oxopentanoate + 5-methylthioadenosine (MTA) + | 0.866055 |
| lanosterol | |
| leucine + 5-methylthioadenosine (MTA) + prolylproline + lanosterol | 0.865819 |
| glycine + 5-methylthioadenosine (MTA) + lanosterol + tauro-beta-muricholate | 0.865584 |

TABLE 4
Top 30 5-variable models

| MODEL | AUC |
|---|---|
| glycine + 4-methyl-2-oxopentanoate + 5-methylthioadenosine (MTA) + | 0.896893 |
| prolylproline + lanosterol | |
| glycine + 3-methyl-2-oxovalerate + 5-methylthioadenosine (MTA) + | 0.896186 |
| prolylproline + lanosterol | |
| 3-methyl-2-oxovalerate + 5-methylthioadenosine (MTA) + prolylproline + | 0.895951 |
| lanosterol + tauro-beta-muricholate | |
| glycine + 3-methyl-2-oxobutyrate + 5-methylthioadenosine (MTA) + | 0.890772 |
| prolylproline + lanosterol | |
| glycine + 3-methyl-2-oxovalerate + 5-methylthioadenosine (MTA) + lanosterol + | 0.889831 |
| tauro-beta-muricholate | |
| 3-methyl-2-oxobutyrate + 5-methylthioadenosine (MTA) + prolylproline + | 0.888889 |
| lanosterol + tauro-beta-muricholate | |
| serine + 3-methyl-2-oxobutyrate + 5-methylthioadenosine (MTA) + | 0.887947 |
| prolylproline + lanosterol | |
| serine + 3-methyl-2-oxovalerate + 5-methylthioadenosine (MTA) + | 0.887476 |
| prolylproline + lanosterol | |
| 3-methyl-2-oxovalerate + 5-methylthioadenosine (MTA) + prolylproline + | 0.886064 |
| lanosterol + deoxycholate | |
| glycine + 5-methylthioadenosine (MTA) + prolylproline + lanosterol + tauro- | 0.885593 |
| beta-muricholate | |
| glycine + leucine + 5-methylthioadenosine (MTA) + prolylproline + lanosterol | 0.885358 |
| 3-methyl-2-oxovalerate + 3-methyl-2-oxobutyrate + 5-methylthioadenosine | 0.884181 |
| (MTA) + prolylproline + lanosterol | |
| 4-methyl-2-oxopentanoate + 5-methylthioadenosine (MTA) + prolylproline + | 0.883239 |
| lanosterol + tauro-beta-muricholate | |
| serine + 3-methyl-2-oxovalerate + 5-methylthioadenosine (MTA) + lanosterol + | 0.882533 |
| tauro-beta-muricholate | |
| glycine + 4-methyl-2-oxopentanoate + 5-methylthioadenosine (MTA) + | 0.882298 |
| lanosterol + tauro-beta-muricholate | |
| serine + 4-methyl-2-oxopentanoate + 5-methylthioadenosine (MTA) + | 0.882062 |
| prolylproline + lanosterol | |
| leucine + 3-methyl-2-oxovalerate + 5-methylthioadenosine (MTA) + | 0.882062 |
| prolylproline + lanosterol | |
| 3-methyl-2-oxobutyrate + 5-methylthioadenosine (MTA) + prolylproline + | 0.881827 |
| lanosterol + deoxycholate | |
| leucine + 3-methyl-2-oxobutyrate + 5-methylthioadenosine (MTA) + | 0.881591 |
| prolylproline + lanosterol | |
| glycine + 3-methyl-2-oxovalerate + 5-methylthioadenosine (MTA) + lanosterol + | 0.88065 |
| deoxycholate | |
| glycine + 2-hydroxybutyrate (AHB) + 5-methylthioadenosine (MTA) + | 0.880414 |
| prolylproline + lanosterol | |
| 4-methyl-2-oxopentanoate + 3-methyl-2-oxobutyrate + 5-methylthioadenosine | 0.880414 |
| (MTA) + prolylproline + lanosterol | |
| 4-methyl-2-oxopentanoate + 5-methylthioadenosine (MTA) + prolylproline + | 0.879944 |
| lanosterol + deoxycholate | |
| 3-methyl-2-oxovalerate + 2-hydroxybutyrate (AHB) + 5-methylthioadenosine | 0.879944 |
| (MTA) + prolylproline + lanosterol | |
| glycine + serine + 3-methyl-2-oxovalerate + 5-methylthioadenosine (MTA) + | 0.879473 |
| lanosterol | |
| leucine + 4-methyl-2-oxopentanoate + 5-methylthioadenosine (MTA) + | 0.879473 |
| prolylproline + lanosterol | |
| valine + 3-methyl-2-oxobutyrate + 5-methylthioadenosine (MTA) + | 0.879473 |
| prolylproline + lanosterol | |
| glycine + serine + 4-methyl-2-oxopentanoate + 5-methylthioadenosine (MTA) + | 0.879237 |
| lanosterol | |
| glycine + valine + 5-methylthioadenosine (MTA) + prolylproline + lanosterol | 0.879237 |
| 3-methyl-2-oxobutyrate + 2-hydroxybutyrate (AHB) + 5-methylthioadenosine | 0.879237 |
| (MTA) + prolylproline + lanosterol | |

TABLE 5
Prevalence of metabolites in the 4- and
5- variable models with an AUC > 0.800.

| Compound | n = 4 | n = 5 |
|---|---|---|
| 5-methylthioadenosine | 92.2% | 93.5% |
| lanosterol | 34.0% | 40.0% |
| glycine | 33.0% | 39.6% |
| 3-methyl-2-oxobutyrate | 29.6% | 35.9% |
| prolylproline | 29.6% | 37.7% |
| 3-methyl-2-oxovalerate | 26.7% | 32.5% |
| serine | 25.2% | 35.3% |
| 4-methyl-2-oxopentanoate | 23.3% | 32.3% |
| valine | 23.3% | 31.4% |
| leucine | 21.8% | 31.4% |
| tauro-beta-muricholate | 21.4% | 29.8% |
| 2-hydroxybutyrate | 20.4% | 30.4% |
| deoxycholate | 19.4% | 30.2% |

### Example 2

### Metabolite Biomarkers of NASH in Human Serum

Serum samples from 116 subjects with NASH, 18 subjects with NAFLD, and 18 subjects with borderline NASH were analyzed using four global metabolic profiling mass spectrometry platforms, as well as the GC-FID analysis for fatty acids, cholesterol metabolism lipids, and Vitamin E. All diagnoses were determined by a trained pathologist using histological analysis of patient biopsy samples. A total of 721 named metabolites were detected in the samples from this cohort. Clinical parameters including Age, Gender, Height/Weight/Body mass index (BMI), Diabetes history, Glucose, Insulin, HBA1c, Aspartate aminotransferase (AST), Alanine aminotransferase (ALT), Total cholesterol, High-density lipoprotein cholesterol (HDL), Low-density lipoprotein cholesterol (LDL), Triglycerides, Gamma-glutamyl transferase (GGT), Steatosis, Lobular Inflammation, Portal Inflammation, Ballooning, and NAFLD Activity Score (NAS) were provided for the subjects.

Logistic regression and area under the curve (AUC) were used to assess the performance of several of the clinical parameters for distinguishing NASH from borderline NASH and NAFLD. The results are shown in Table 6.

TABLE 6
AUC values for select clinical parameters

| Clinical Parameter | AUC |
|---|---|
| NAS | 0.905 |
| AST | 0.706 |
| Insulin | 0.671 |
| ALT | 0.620 |
| BMI | 0.611 |
| HbA1c | 0.593 |
| HDL | 0.589 |
| GGT | 0.578 |
| Age | 0.547 |
| Glucose | 0.539 |
| Cholesterol | 0.529 |
| LDL | 0.525 |
| Triglycerides | 0.520 |

All 721 named metabolites were analyzed using a mixed effects model. Metabolites that were significantly altered (p<0.05, q<0.1) in the comparison of NASH to NAFLD samples are presented in Table 7. Other comparisons presented in Table 7 are Baseline (BL) NASH vs. NAFLD, and NASH vs. BL NASH. Table 7 includes, for each metabolite, the biochemical name of the metabolite, the internal identifier for the biomarker compound in the in-house chemical library of authentic standards (CompID), the fold change (FC) of the biomarker for each comparison, which is the ratio of the mean level of the biomarker in one sample type as compared to the mean level in a different sample type (e.g. NASH versus NAFLD), and the p-value determined in the statistical analysis of the data concerning the biomarkers.

TABLE 7
Metabolite biomarkers in comparisons of NASH, BL NASH, and
NAFLD samples.

| | | NASH/ | | BL NASH/ | | NASH/BL | |
|---|---|---|---|---|---|---|---|
| | Comp | NAFLD | | NAFLD | | NASH | |
| Biochemical Name | ID | FC | p-value | FC | p-value | FC | p-value |
| epiandrosterone sulfate | 33973 | 0.55 | 1.42E−05 | 0.7 | 0.0728 | 0.79 | 0.4457 |
| androsterone sulfate | 31591 | 0.61 | 4.86E−05 | 0.76 | 0.0849 | 0.79 | 0.5539 |
| I-urobilinogen | 32426 | 7.03 | 0.0088 | 4.74 | 0.3162 | 1.48 | 0.4613 |
| 16-hydroxypalmitate | 39609 | 1.35 | 0.0013 | 1.15 | 0.1749 | 1.17 | 0.0561 |
| 3-hydroxyoctanoate | 22001 | 1.58 | 0.006 | 1.34 | 0.248 | 1.17 | 0.4393 |
| dehydroisoandrosterone | 32425 | 0.65 | 0.0008 | 0.82 | 0.1463 | 0.79 | 0.541 |
| sulfate (DHEA-S) | | | | | | | |
| 5-methylthioadenosine | | 1.81 | 0.02679 | | | 1.43 | 0.11437 |
| (MTA) | | | | | | | |
| valylglycine | 40475 | 0.47 | 0.0007 | 0.78 | 0.3215 | 0.6 | 0.0439 |
| cyclo(L-phe-L-pro) | 44875 | 2.8 | 0.0042 | 1.21 | 0.8317 | 2.31 | 0.0133 |
| TL16:1n7 (palmitoleic acid) | 48798 | 1.4 | 0.0003 | 1.42 | 0.0071 | 0.98 | 0.9867 |
| palmitoleate (16:1n7) | 33447 | 1.58 | 0.0047 | 1.6 | 0.0386 | 0.98 | 0.9292 |
| TL16:0 (palmitic acid) | 48792 | 1.07 | 0.0018 | 1.02 | 0.5393 | 1.05 | 0.0255 |
| isoleucylglycine | 40008 | 0.53 | 1.94E−10 | 1 | 0.8351 | 0.52 | 6.46E−08 |
| 1- | 44682 | 0.76 | 8.04E−05 | 0.85 | 0.0596 | 0.9 | 0.4609 |
| margaroylglycerophosphocholine | | | | | | | |
| (17:0) | | | | | | | |
| glycerol | 15122 | 1.56 | 0.0002 | 1.45 | 0.0055 | 1.08 | 0.7537 |
| 5alpha-androstan- | 37192 | 0.54 | 0.0003 | 0.74 | 0.1478 | 0.73 | 0.2129 |
| 3beta,17beta-diol | | | | | | | |
| monosulfate | | | | | | | |
| hydroxybutyrylcarnitine | 43264 | 1.75 | 0.0003 | 1.2 | 0.497 | 1.46 | 0.0392 |
| caprate (10:0) | 1642 | 1.3 | 0.0004 | 1.02 | 0.9104 | 1.28 | 0.0033 |
| 4-androsten-3alpha,17alpha- | 37209 | 0.73 | 0.0006 | 0.78 | 0.075 | 0.93 | 0.7875 |
| diol monosulfate | | | | | | | |
| 4-androsten-3beta,17beta-diol | 37211 | 0.61 | 0.0006 | 0.79 | 0.1702 | 0.78 | 0.3384 |
| monosulfate | | | | | | | |
| 5alpha-androstan- | 37187 | 0.66 | 0.0006 | 0.63 | 0.0523 | 1.05 | 0.8537 |
| 3beta,17alpha-diol disulfate | | | | | | | |
| isoleucylvaline | 40049 | 0.7 | 0.0006 | 0.94 | 0.7747 | 0.74 | 0.0025 |
| 1- | 45456 | 0.55 | 0.0008 | 0.89 | 0.3435 | 0.62 | 0.1665 |
| arachidoylglycerophosphocholine | | | | | | | |
| (20:0) | | | | | | | |
| hypoxanthine | 3127 | 0.83 | 0.0009 | 1.12 | 0.1671 | 0.75 | 5.67E−06 |
| N-acetylmethionine | 1589 | 0.83 | 0.0015 | 1.07 | 0.5666 | 0.77 | 0.0002 |
| 3-hydroxybutyrate (BHBA) | 542 | 2 | 0.0016 | 1.05 | 0.6822 | 1.9 | 0.0075 |
| cyclohexanebutanoic acid | 48776 | 1.62 | 0.0016 | 1.29 | 0.1212 | 1.26 | 0.2151 |
| 5alpha-pregnan- | 37200 | 0.75 | 0.0017 | 10.11 | 0.7814 | 0.07 | 0.0576 |
| 3beta,20alpha-diol | | | | | | | |
| monosulfate (2) | | | | | | | |
| myristoleate (14:1n5) | 32418 | 1.71 | 0.0017 | 1.5 | 0.0685 | 1.15 | 0.6199 |
| pregn steroid monosulfate | 32619 | 0.59 | 0.0018 | 0.7 | 0.0917 | 0.84 | 0.6145 |
| myristate (14:0) | 1365 | 1.31 | 0.002 | 1.19 | 0.2867 | 1.1 | 0.2985 |
| valylleucine | 39994 | 0.86 | 0.002 | 1.33 | 0.9075 | 0.65 | 0.0638 |
| 21-hydroxypregnenolone | 46115 | 0.78 | 0.0023 | 0.99 | 0.3019 | 0.79 | 0.5101 |
| disulfate | | | | | | | |
| laurate (12:0) | 1645 | 1.36 | 0.0023 | 1.14 | 0.5484 | 1.19 | 0.2259 |
| 2- | 48259 | 0.79 | 0.003 | 0.95 | 0.5983 | 0.83 | 0.0125 |
| oleoylglycerophosphocholine | | | | | | | |
| phenylalanylvaline | 41393 | 0.48 | 0.0032 | 0.77 | 0.2671 | 0.62 | 0.2273 |
| 10-heptadecenoate (17:1n7) | 33971 | 1.41 | 0.0036 | 1.39 | 0.1116 | 1.01 | 0.7728 |
| catechol sulfate | 35320 | 0.73 | 0.0041 | 0.92 | 0.2475 | 0.79 | 0.3594 |
| xanthine | 3147 | 1.34 | 0.0044 | 1.16 | 0.0742 | 1.16 | 0.2822 |
| 2- | 34258 | 0.81 | 0.0051 | 0.84 | 0.1479 | 0.97 | 0.8186 |
| docosahexaenoylglycerophosphoethanolamine | | | | | | | |
| glutarylcarnitine (C5) | 44664 | 0.79 | 0.0051 | 0.85 | 0.1547 | 0.93 | 0.3914 |
| pregnanediol-3-glucuronide | 40708 | 0.76 | 0.0052 | 3.61 | 0.8622 | 0.21 | 0.0782 |
| pregnenolone sulfate | 38170 | 0.69 | 0.0064 | 0.89 | 0.2264 | 0.77 | 0.5967 |
| 5-dodecenoate (12:1n7) | 33968 | 1.59 | 0.0066 | 1.2 | 0.3041 | 1.32 | 0.2053 |
| 1- | 44563 | 0.51 | 0.0069 | 0.66 | 0.2206 | 0.77 | 0.1912 |
| eicosapentaenoylglycerophosphocholine | | | | | | | |
| (20:5n3) | | | | | | | |
| malate | 1303 | 1.3 | 0.0069 | 1.13 | 0.3127 | 1.15 | 0.1207 |
| docosatrienoate (22:3n3) | 32417 | 1.41 | 0.0071 | 1.1 | 0.7902 | 1.29 | 0.0468 |
| leucylglycine | 40045 | 0.92 | 0.0073 | 1.7 | 0.2712 | 0.54 | 0.0037 |
| biliverdin | 2137 | 0.74 | 0.0087 | 0.77 | 0.1179 | 0.96 | 0.6056 |
| dodecanedioate | 32388 | 1.24 | 0.0095 | 1.25 | 0.0514 | 0.99 | 0.7643 |
| 1- | 44633 | 0.8 | 0.0104 | 0.84 | 0.1075 | 0.95 | 0.855 |
| docosahexaenoylglycerophosphoethanolamine | | | | | | | |
| 3-methoxytyramine sulfate | 44618 | 1.3 | 0.0104 | 1.3 | 0.0885 | 1 | 0.9362 |
| 1- | 35628 | 0.8 | 0.0114 | 0.97 | 0.8286 | 0.82 | 0.0235 |
| oleoylglycerophosphoethanolamine | | | | | | | |
| carnitine | 15500 | 0.89 | 0.012 | 1 | 0.9666 | 0.9 | 0.0091 |

Logistic regression models and area under the curve (AUC) were used to assess how well individual metabolites distinguished the NASH from borderline NASH and NAFLD groups. Logistic regression analysis was performed for all 721 named metabolites. Metabolites with an AUC of >0.620 for distinguishing NASH from borderline NASH and NAFLD patient samples are presented in Table 8. Metabolites in bold are significant with p<0.05, q<0.1 in NASH compared to NAFLD patient samples.

TABLE 8
AUC of individual metabolites for distinguishing
NASH from borderline NASH and NAFLD

| Biochemical Name | AUC | Biochemical Name | AUC |
|---|---|---|---|
| epiandrosterone sulfate | 0.677 | myristate (14:0) | 0.651 |
| androsterone sulfate | 0.661 | 2′-deoxyuridine | 0.651 |
| 16-hydroxypalmitate | 0.71 | 3-methyl-2-oxobutyrate | 0.649 |
| fucose | 0.653 | 1- | 0.648 |
| | | oleoylglycerophosphoethanolamine | |
| taurine | 0.665 | threonylphenylalanine | 0.648 |
| 3-hydroxyoctanoate | 0.626 | adrenate (22:4n6) | 0.647 |
| dehydroisoandrosterone sulfate | 0.654 | 3-(4-hydroxyphenyl)lactate | 0.646 |
| (DHEA-S) | | | |
| 5-methylthioadenosine (MTA) | 0.635 | 5-methyluridine (ribothymidine) | 0.645 |
| gamma-glutamylhistidine | 0.684 | pregnanediol-3-glucuronide | 0.643 |
| valylglycine | 0.719 | 15-methylpalmitate (isobar with 2- | 0.642 |
| | | methylpalmitate) | |
| 3-hydroxyisobutyrate | 0.708 | glycerol 3-phosphate (G3P) | 0.642 |
| cyclo(L-phe-L-pro) | 0.731 | 10-nonadecenoate (19:1n9) | 0.641 |
| taurocholate | 0.627 | eicosanodioate | 0.641 |
| TL16:1n7 (palmitoleic acid) | 0.631 | 1-linolenoylglycerophosphocholine | 0.641 |
| | | (18:3n3) | |
| TL16:0 (palmitic acid) | 0.692 | leucylalanine | 0.64 |
| palmitate (16:0) | 0.653 | guanosine | 0.638 |
| isoleucylglycine | 0.804 | 1-palmitoylplasmenylethanolamine | 0.638 |
| hypoxanthine | 0.728 | 1- | 0.637 |
| | | arachidoylglycerophosphocholine | |
| | | (20:0) | |
| 2-oleoylglycerophosphocholine | 0.705 | xanthine | 0.637 |
| phenylalanylvaline | 0.696 | N-methylproline | 0.636 |
| valylleucine | 0.694 | 1,6-anhydroglucose | 0.636 |
| isoleucylvaline | 0.694 | phenylalanylleucine | 0.636 |
| 5alpha-pregnan-3beta,20alpha- | 0.689 | glycerol | 0.635 |
| diol monosulfate | | | |
| 4-androsten-3alpha,17alpha-diol | 0.687 | threonate | 0.635 |
| monosulfate | | | |
| scyllo-inositol | 0.687 | 1-linoleoylglycerophosphocholine | 0.633 |
| | | (18:2n6) | |
| hydroxybutyrylcarnitine | 0.684 | etiocholanolone glucuronide | 0.633 |
| docosatrienoate (22:3n3) | 0.681 | uridine | 0.633 |
| alpha-hydroxyisovalerate | 0.679 | oxalate (ethanedioate) | 0.632 |
| tryptophylleucine | 0.678 | 5-dodecenoate (12:1n7) | 0.631 |
| cyclo(leu-pro) | 0.678 | 10-heptadecenoate (17:1n7) | 0.631 |
| cysteine | 0.674 | myristoleate (14:1n5) | 0.631 |
| 3-hydroxybutyrate (BHBA) | 0.672 | oleoyl-linoleoyl- | 0.63 |
| | | glycerophosphocholine | |
| malate | 0.672 | N-acetylvaline | 0.63 |
| maleate (cis-Butenedioate) | 0.669 | pregnenolone sulfate | 0.63 |
| malonate (propanedioate) | 0.668 | glutarylcarnitine (C5) | 0.629 |
| phenylalanylglycine | 0.668 | stearate (18:0) | 0.629 |
| caprate (10:0) | 0.668 | 21-hydroxypregnenolone disulfate | 0.628 |
| 1- | 0.667 | stearoyl-linoleoyl- | 0.627 |
| margaroylglycerophosphocholine | | glycerophosphocholine | |
| (17:0) | | | |
| N-acetylmethionine | 0.666 | o-cresol sulfate | 0.627 |
| carnitine | 0.663 | 2-hydroxyglutarate | 0.625 |
| 4-androsten-3beta,17beta-diol | 0.661 | 2-hydroxy-30-methylvalerate | 0.625 |
| monosulfate | | | |
| leucylleucine | 0.659 | alpha-ketobutyrate | 0.625 |
| leucylglycine | 0.659 | 1- | 0.623 |
| | | eicosapentaenoylglycerophosphocholine | |
| | | (20:5n3) | |
| cyclohexanebutanoic acid | 0.659 | catechol sulfate | 0.623 |
| pregn steroid monosulfate | 0.657 | serylalanine | 0.623 |
| methyl glucopyranoside (alpha + | 0.654 | 1- | 0.621 |
| beta) | | arachidonoylglycerophosphocholine | |
| | | (20:4n6) | |
| 5alpha-androstan-3beta,17beta- | 0.653 | 1- | 0.621 |
| diol monosulfate | | nonadecanoylglycerophosphocholine | |
| | | (19:0) | |
| inosine | 0.652 | betonicine | 0.62 |

### Example 3

### Metabolite Biomarkers of Fibrosis in Human Serum

Serum samples from 152 subjects with liver biopsy-diagnosed NASH or NAFLD were used in the analysis. All diagnoses were determined by a trained pathologist using histological analysis of patient biopsy samples. Patient samples were classified into three groups according to disease severity based on the fibrosis stage (stage 0, least severe; stage 1-2, moderate severity; stage 3-4, high severity). All samples were analyzed using four global metabolic profiling mass spectrometry platforms, as well as the GC-FID analysis for fatty acids, cholesterol metabolism lipids, and Vitamin E. A total of 721 named metabolites were detected in the sample cohort. Clinical parameters including Age, Gender, Height/Weight/Body mass index (BMI), Diabetes history, Glucose, Insulin, HBA1c, Aspartate aminotransferase (AST), Alanine aminotransferase (ALT), Total cholesterol, High-density lipoprotein cholesterol (HDL), Low-density lipoprotein cholesterol (LDL), Triglycerides, Gamma-glutamyl transferase (GGT), Steatosis, Lobular Inflammation, Portal Inflammation, Ballooning, and NAFLD Activity Score (NAS) were provided for the subjects.

Logistic regression and area under the curve (AUC) were used to assess the performance of several of the clinical parameters for distinguishing fibrosis stages 3-4 (high severity) from stages 1-2 (moderate severity) and stage 0 (low severity). The results are shown in Table 9.

TABLE 9
AUC values for select clinical parameters

| Clinical Parameter | AUC |
|---|---|
| GGT | 0.712 |
| AST | 0.71 |
| HbA1c | 0.663 |
| Age | 0.66 |
| BMI | 0.658 |
| NAS | 0.59 |
| Cholesterol | 0.578 |
| Triglycerides | 0.571 |
| Insulin | 0.57 |
| LDL | 0.57 |
| ALT | 0.537 |
| Glucose | 0.534 |
| HDL | 0.507 |

The measured levels of the 721 named metabolites detected in the samples were analyzed using a mixed effects model. Metabolites that were significantly altered (p<0.05, q<0.1) in the comparison of Stage 3+4 (high severity) fibrosis to Stage 0 (low severity) fibrosis samples are presented in Table 10. Other comparisons presented in Table 10 are Stage 3+4 (high severity) vs. Stage 1+2 (moderate severity), and Stage 1+2 vs. Stage 0. Table 10 includes, for each metabolite, the biochemical name of the metabolite, the internal identifier for the biomarker compound in the in-house chemical library of authentic standards (ComplD), the fold change (FC) of the biomarker for each comparison, which is the ratio of the mean level of that biomarker in one sample type as compared to the mean level in a different sample type, and the p-value determined in the statistical analysis of the data concerning the biomarkers.

TABLE 10
Biomarkers of Fibrosis and the Stage of Fibrosis.

| | | STAGE | | STAGE | | | |
|---|---|---|---|---|---|---|---|
| | | 3 + 4/ | | 3 + 4/ | | STAGE 1 + 2/ | |
| | Comp | Stage 0 | | Stage 1 + 2 | | Stage 0 | |
| Biochemical Name | ID | FC | p-value | FC | p-value | FC | p-value |
| epiandrosterone sulfate | 33973 | 0.31 | 5.87E−06 | 0.51 | 0.0003 | 0.6 | 0.0011 |
| androsterone sulfate | 31591 | 0.32 | 7.62E−06 | 0.54 | 0.0005 | 0.59 | 0.001 |
| I-urobilinogen | 32426 | 6.77 | 0.0079 | 4.4 | 0.0855 | 1.54 | 0.0549 |
| 16-hydroxypalmitate | 39609 | 1.41 | 0.0001 | 1.22 | 0.0176 | 1.15 | 0.0105 |
| fucose | 15821 | 1.58 | 7.24E−05 | 1.51 | 0.0002 | 1.05 | 0.7392 |
| taurine | 2125 | 0.72 | 7.39E−06 | 0.83 | 0.0056 | 0.87 | 0.0035 |
| 3-hydroxydecanoate | 22053 | 1.52 | 0.0219 | 1.1 | 0.5042 | 1.38 | 0.0071 |
| 3-hydroxyoctanoate | 22001 | 1.62 | 0.0244 | 1 | 0.727 | 1.62 | 0.0017 |
| 16a-hydroxy DHEA 3-sulfate | 38168 | 1.77 | 0.0037 | 1.54 | 0.0224 | 1.15 | 0.4092 |
| dehydroisoandrosterone sulfate | 32425 | 0.32 | 1.27E−05 | 0.53 | 0.0025 | 0.61 | 0.0051 |
| (DHEA-S) | | | | | | | |
| 5-methylthioadenosine (MTA) | | 1.93 | 0.00022 | 1.28 | 0.1176 | | |
| gamma-glutamylhistidine | 18245 | 1.52 | 0.0018 | 1.12 | 0.3935 | 1.35 | 0.0014 |
| valylglycine | 40475 | 0.51 | 0.0109 | 0.89 | 0.6421 | 0.57 | 0.0039 |
| cyclo(L-phe-L-pro) | 44875 | 2.46 | 0.0057 | 2.1 | 0.0854 | 1.17 | 0.0479 |
| taurocholate | 18497 | 7.57 | 0.0007 | 6.99 | 0.0018 | 1.08 | 0.5309 |
| glycocholate | 18476 | 3.61 | 0.0002 | 4.07 | 0.0004 | 0.89 | 0.7232 |
| taurochenodeoxycholate | 18494 | 13 | 0.0026 | 9.89 | 0.0041 | 1.32 | 0.7243 |
| glycochenodeoxycholate | 32346 | 3.81 | 0.0003 | 4.38 | 0.0003 | 0.87 | 0.9724 |
| glutamate | 57 | 1.21 | 0.022 | 1.05 | 0.3109 | 1.16 | 0.162 |
| palmitoleate (16:1n7) | 33447 | 1.67 | 5.60E−05 | 1.55 | 0.0005 | 1.08 | 0.3881 |
| TL16:0 (palmitic acid) | 48792 | 1.07 | 0.0016 | 1.03 | 0.1505 | 1.04 | 0.0174 |
| 5alpha-androstan- | 37192 | 0.29 | 4.28E−07 | 0.58 | 0.0006 | 0.5 | 0.0002 |
| 3beta,17beta-diol monosulfate | | | | | | | |
| pregnanediol-3-glucuronide | 40708 | 0.18 | 4.40E−06 | 0.43 | 0.0001 | 0.42 | 0.0381 |
| 5alpha-pregnan-3beta,20alpha- | 37200 | 0.08 | 7.20E−06 | 0.43 | 0.0059 | 0.19 | 0.0007 |
| diol monosulfate | | | | | | | |
| 5alpha-androstan- | 37186 | 0.24 | 1.19E−05 | 0.43 | 0.0014 | 0.56 | 0.0943 |
| 3alpha,17beta-diol | | | | | | | |
| monosulfate | | | | | | | |
| tryptophylleucine | 40080 | 0.42 | 3.25E−05 | 0.55 | 0.0069 | 0.77 | 0.0249 |
| 4-androsten-3beta,17beta-diol | 37211 | 0.31 | 4.03E−05 | 0.53 | 0.004 | 0.58 | 0.0029 |
| monosulfate | | | | | | | |
| 5alpha-androstan- | 37190 | 0.41 | 0.0001 | 0.51 | 0.0005 | 0.82 | 0.31 |
| 3beta,17beta-diol disulfate | | | | | | | |
| phenylalanylvaline | 41393 | 0.29 | 0.0001 | 0.52 | 0.0107 | 0.57 | 0.0296 |
| 4-androsten-3alpha,17alpha- | 37209 | 0.43 | 0.0002 | 0.62 | 0.0035 | 0.69 | 0.0237 |
| diol monosulfate | | | | | | | |
| etiocholanolone glucuronide | 47112 | 0.37 | 0.0002 | 0.49 | 0.0003 | 0.74 | 0.4714 |
| isoleucylglycine | 40008 | 0.45 | 0.0002 | 0.75 | 0.2434 | 0.6 | 0.0001 |
| isoleucylvaline | 40049 | 0.42 | 0.0002 | 0.57 | 0.0926 | 0.75 | 0.0011 |
| uridine | 606 | 0.86 | 0.0002 | 0.93 | 0.1212 | 0.93 | 0.0136 |
| 5alpha-androstan- | 37187 | 0.55 | 0.0003 | 0.89 | 0.0046 | 0.61 | 0.0358 |
| 3beta,17alpha-diol disulfate | | | | | | | |
| myristoleate (14:1n5) | 32418 | 1.8 | 0.0003 | 1.59 | 0.0029 | 1.13 | 0.347 |
| pregn steroid monosulfate | 32619 | 0.44 | 0.0003 | 0.71 | 0.0707 | 0.61 | 0.0017 |
| valylleucine | 39994 | 0.39 | 0.0003 | 0.44 | 0.0276 | 0.89 | 0.0277 |
| xanthurenate | 15679 | 0.39 | 0.0003 | 0.42 | 0.0005 | 0.92 | 0.7952 |
| ADSGEGDFXAEGGGVR | 33084 | 1.64 | 0.0004 | 1.28 | 0.0133 | 1.27 | 0.2566 |
| serine | 1648 | 1.22 | 0.0004 | 1.12 | 0.0244 | 1.09 | 0.1271 |
| theanine | 22206 | 0.07 | 0.0005 | 0.11 | 0.0035 | 0.65 | 0.664 |
| alpha-hydroxyisovalerate | 33937 | 1.45 | 0.0006 | 1.39 | 0.0023 | 1.04 | 0.4508 |
| pyridoxate | 31555 | 0.26 | 0.0007 | 0.55 | 0.031 | 0.48 | 0.0853 |
| docosatrienoate (22:3n3) | 32417 | 1.42 | 0.0009 | 1.21 | 0.0409 | 1.17 | 0.0918 |
| 10-heptadecenoate (17:1n7) | 33971 | 1.43 | 0.001 | 1.31 | 0.0094 | 1.09 | 0.3403 |
| isovalerylglycine | 35107 | 0.7 | 0.0014 | 0.76 | 0.0197 | 0.92 | 0.2048 |
| 7-alpha-hydroxy-3-oxo-4- | 36776 | 1.39 | 0.0015 | 1.45 | 0.0005 | 0.96 | 0.4887 |
| cholestenoate (7-Hoca) | | | | | | | |
| glycerol | 15122 | 1.49 | 0.0015 | 1.35 | 0.0109 | 1.1 | 0.3094 |
| hypoxanthine | 3127 | 0.63 | 0.0015 | 0.85 | 0.2255 | 0.75 | 2.86E−05 |
| glycyltryptophan | 38151 | 0.56 | 0.0018 | 0.58 | 0.0027 | 0.98 | 0.8778 |
| inosine | 1123 | 0.42 | 0.0021 | 0.63 | 0.2525 | 0.66 | 0.0038 |
| 5,6-dihydrothymine | 1418 | 1.5 | 0.0022 | 1.57 | 0.0006 | 0.96 | 0.327 |
| malate | 1303 | 1.36 | 0.0022 | 1.14 | 0.1548 | 1.19 | 0.0062 |
| N-acetylcarnosine | 43488 | 0.69 | 0.0024 | 0.7 | 0.0064 | 0.98 | 0.6376 |
| 5-dodecenoate (12:1n7) | 33968 | 1.91 | 0.0025 | 1.58 | 0.014 | 1.21 | 0.4188 |
| tauro-beta-muricholate | 33983 | 11.69 | 0.0029 | 6.07 | 0.0164 | 1.93 | 0.1987 |
| 2- | 35257 | 0.78 | 0.0031 | 0.93 | 0.2649 | 0.84 | 0.0023 |
| linoleoylglycerophosphocholine | | | | | | | |
| ergothioneine | 37459 | 0.63 | 0.0032 | 0.85 | 0.0228 | 0.75 | 0.071 |
| 5-methyluridine | 35136 | 0.88 | 0.0033 | 0.93 | 0.1568 | 0.94 | 0.0374 |
| (ribothymidine) | | | | | | | |
| cis-vaccenate (18:1n7) | 33970 | 1.38 | 0.0034 | 1.27 | 0.02 | 1.09 | 0.3785 |
| nicotinamide | 594 | 0.69 | 0.0037 | 0.59 | 0.0389 | 1.17 | 0.4404 |
| xylitol | 4966 | 1.53 | 0.0038 | 1.25 | 0.1618 | 1.23 | 0.0149 |
| malonate (propanedioate) | 15872 | 1.28 | 0.0039 | 1.11 | 0.2588 | 1.15 | 0.0074 |
| 1- | 44621 | 0.66 | 0.004 | 0.84 | 0.2622 | 0.79 | 0.0138 |
| oleoylplasmenylethanolamine | | | | | | | |
| 7-methylguanine | 35114 | 1.18 | 0.004 | 1.08 | 0.1101 | 1.1 | 0.1208 |
| alliin | 41494 | 0.36 | 0.0041 | 0.48 | 0.0918 | 0.75 | 0.0813 |
| 3-methylglutarylcarnitine | 46547 | 1.43 | 0.0042 | 1.03 | 0.4483 | 1.39 | 0.0069 |
| N2-methylguanosine | 35133 | 1.22 | 0.0044 | 1.18 | 0.012 | 1.03 | 0.6387 |
| sorbitol | 15053 | 1.72 | 0.0046 | 1.45 | 0.0259 | 1.19 | 0.2653 |
| phenylalanylglycine | 41370 | 0.58 | 0.0047 | 0.8 | 0.1813 | 0.72 | 0.0143 |
| TL16:1n7 (palmitoleic acid) | 48798 | 1.19 | 0.0047 | 1.05 | 0.2829 | 1.13 | 0.0315 |
| TL18:1n7 (avaccenic acid) | 48799 | 1.14 | 0.0049 | 1.07 | 0.1664 | 1.07 | 0.0092 |
| hydroxybutyrylcarnitine | 43264 | 1.76 | 0.005 | 1.4 | 0.1011 | 1.25 | 0.0338 |
| scyllo-inositol | 32379 | 0.68 | 0.005 | 0.74 | 0.0403 | 0.92 | 0.2233 |
| N-palmitoyl glycine | 42092 | 1.4 | 0.0051 | 1.46 | 0.0028 | 0.96 | 0.7616 |
| isobutyrylglycine | 35437 | 0.74 | 0.0055 | 0.75 | 0.0197 | 0.99 | 0.5413 |
| adrenate (22:4n6) | 32980 | 1.35 | 0.0057 | 1.22 | 0.0718 | 1.11 | 0.1285 |
| tyramine O-sulfate | 48408 | 0.47 | 0.0057 | 0.97 | 0.0287 | 0.48 | 0.4445 |
| pregnenolone sulfate | 38170 | 0.55 | 0.0062 | 0.86 | 0.502 | 0.64 | 0.0018 |
| leucylleucine | 36756 | 0.66 | 0.0063 | 0.81 | 0.5802 | 0.82 | 0.0061 |
| phenylacetylcarnitine | 48425 | 1.92 | 0.0064 | 1.47 | 0.0905 | 1.3 | 0.1363 |
| 1- | 44563 | 0.56 | 0.0067 | 0.66 | 0.1019 | 0.85 | 0.1143 |
| eicosapentaenoylglycerophosphocholine | | | | | | | |
| (20:5n3) | | | | | | | |
| cyclohexanebutanoic acid | 48776 | 1.73 | 0.0071 | 1.33 | 0.1644 | 1.3 | 0.0282 |
| palmitate (16:0) | 1336 | 1.14 | 0.0076 | 1.1 | 0.0549 | 1.04 | 0.3236 |
| 1- | 44682 | 0.8 | 0.0082 | 0.91 | 0.2296 | 0.88 | 0.0207 |
| margaroylglycerophosphocholine | | | | | | | |
| (17:0) | | | | | | | |
| myristate (14:0) | 1365 | 1.22 | 0.0082 | 1.14 | 0.0618 | 1.07 | 0.2853 |
| phenylacetate | 15958 | 1.38 | 0.0084 | 1.25 | 0.2351 | 1.11 | 0.029 |
| arabinose | 575 | 1.31 | 0.0086 | 1.18 | 0.0538 | 1.11 | 0.3997 |
| gamma-glutamylleucine | 18369 | 0.82 | 0.0095 | 0.81 | 0.0013 | 1.01 | 0.4517 |
| N-oleoyltaurine | 39732 | 1.96 | 0.0098 | 1.85 | 0.0027 | 1.06 | 0.3723 |
| 1- | 34061 | 0.79 | 0.01 | 0.85 | 0.0529 | 0.93 | 0.2319 |
| arachidonoylglycerophosphocholine | | | | | | | |
| (20:4n6) | | | | | | | |
| 4-androsten-3beta,17beta-diol | 37202 | 0.59 | 0.0102 | 0.64 | 0.0357 | 0.91 | 0.3385 |
| disulfate | | | | | | | |
| suberate (octanedioate) | 15730 | 0.77 | 0.0106 | 0.83 | 0.2659 | 0.93 | 0.153 |
| N-acetylmethionine | 1589 | 0.78 | 0.0109 | 1.02 | 0.5751 | 0.76 | 0.0002 |
| N1-Methyl-2-pyridone-5- | 40469 | 0.72 | 0.0111 | 0.63 | 0.0356 | 1.15 | 0.7019 |
| carboxamide | | | | | | | |
| docosadienoate (22:2n6) | 32415 | 1.21 | 0.0113 | 1.16 | 0.0279 | 1.04 | 0.7524 |
| indole-3-carboxylic acid | 38116 | 1.38 | 0.0113 | 1.2 | 0.1573 | 1.15 | 0.1229 |
| TL20:5n3 (eicosapentaenoic | 48816 | 0.65 | 0.0119 | 0.73 | 0.0865 | 0.88 | 0.2709 |
| acid) | | | | | | | |
| piperine | 33935 | 0.46 | 0.012 | 0.64 | 0.081 | 0.72 | 0.2392 |
| stigmasterol | 45499 | 1.42 | 0.0122 | 1.43 | 0.0028 | 0.99 | 0.4191 |
| leucylglycine | 40045 | 0.59 | 0.0125 | 0.74 | 0.4031 | 0.8 | 0.009 |
| 2- | 48259 | 0.83 | 0.0127 | 0.96 | 0.8894 | 0.86 | 0.0114 |
| oleoylglycerophosphocholine | | | | | | | |
| gamma-glutamyltyrosine | 2734 | 1.31 | 0.0127 | 1.21 | 0.0899 | 1.08 | 0.1248 |
| palmitoylcarnitine | 44681 | 1.42 | 0.0132 | 1.47 | 0.0038 | 0.97 | 0.3971 |
| fumarate | 1643 | 1.24 | 0.0134 | 1.2 | 0.0237 | 1.03 | 0.7979 |
| 4-allylphenol sulfate | 37181 | 0.48 | 0.0135 | 0.56 | 0.2315 | 0.85 | 0.1264 |
| myristoleoylcarnitine | 48182 | 1.63 | 0.0137 | 1.44 | 0.0184 | 1.13 | 0.9751 |
| 1- | 34419 | 0.78 | 0.0138 | 0.95 | 0.6177 | 0.82 | 0.0042 |
| linoleoylglycerophosphocholine | | | | | | | |
| (18:2n6) | | | | | | | |
| 4-guanidinobutanoate | 15681 | 0.6 | 0.0145 | 0.63 | 0.004 | 0.95 | 0.705 |
| 1- | 39270 | 0.72 | 0.0146 | 0.79 | 0.079 | 0.9 | 0.2601 |
| palmitoylplasmenylethanolamine | | | | | | | |
| 10-nonadecenoate (19:1n9) | 33972 | 1.25 | 0.0147 | 1.13 | 0.1825 | 1.11 | 0.1333 |
| valerate | 33443 | 1.22 | 0.015 | 1.21 | 0.02 | 1.01 | 0.8939 |
| urate | 1604 | 0.86 | 0.0159 | 0.86 | 0.0249 | 0.99 | 0.7664 |
| dopamine sulfate | 48406 | 1.12 | 0.0162 | 0.87 | 0.3111 | 1.28 | 0.1256 |
| 21-hydroxypregnenolone | 46115 | 0.68 | 0.0171 | 0.85 | 0.1504 | 0.8 | 0.0669 |
| disulfate | | | | | | | |
| TL18:3n6 (g-linolenic acid) | 48806 | 0.74 | 0.0175 | 0.84 | 0.157 | 0.88 | 0.1712 |
| 3-(4-hydroxyphenyl)lactate | 32197 | 1.31 | 0.0181 | 1.21 | 0.0961 | 1.08 | 0.2041 |
| eicosenoate (20:1n9 or 11) | 33587 | 1.27 | 0.0183 | 1.19 | 0.0588 | 1.07 | 0.5506 |
| palmitoyl ethanolamide | 38165 | 1.11 | 0.0183 | 1.11 | 0.0186 | 1 | 0.893 |
| 1- | 45951 | 0.68 | 0.0187 | 0.92 | 0.6393 | 0.73 | 0.0092 |
| linolenoylglycerophosphocholine | | | | | | | |
| (18:3n3) | | | | | | | |
| histidine | 59 | 0.87 | 0.0188 | 0.95 | 0.1617 | 0.91 | 0.0985 |
| 1-linolenoylglycerol | 34393 | 0.74 | 0.0189 | 0.8 | 0.1149 | 0.93 | 0.2253 |
| mannose | 48153 | 1.3 | 0.0192 | 1.15 | 0.3083 | 1.12 | 0.0081 |
| myristoylcarnitine | 33952 | 1.65 | 0.0194 | 1.42 | 0.0494 | 1.16 | 0.658 |
| valine | 1649 | 0.86 | 0.0197 | 0.88 | 0.0214 | 0.98 | 0.8514 |
| pantothenate | 1508 | 0.71 | 0.0202 | 0.77 | 0.0884 | 0.92 | 0.3954 |
| pimelate (heptanedioate) | 15704 | 0.75 | 0.0212 | 0.78 | 0.0619 | 0.97 | 0.412 |
| thyroxine | 46079 | 1.62 | 0.0216 | 1.49 | 0.0685 | 1.09 | 0.3924 |
| 1- | 37231 | 0.72 | 0.0217 | 0.77 | 0.0387 | 0.93 | 0.6217 |
| docosapentaenoylglycerophosphocholine | | | | | | | |
| (22:5n3) | | | | | | | |
| glycolithocholate | 31912 | 4.26 | 0.0222 | 3.74 | 0.0767 | 1.14 | 0.2914 |
| proline | 1898 | 1.21 | 0.0224 | 1.04 | 0.4474 | 1.16 | 0.044 |
| 5alpha-pregnan-3beta,20alpha- | 37198 | 0.3 | 0.0227 | 0.63 | 0.279 | 0.48 | 0.1064 |
| diol disulfate | | | | | | | |
| 5-pregnen-3b, 17-diol-20-one | 37482 | 0.52 | 0.0231 | 0.82 | 0.7805 | 0.63 | 0.0053 |
| 3-sulfate | | | | | | | |
| glutarylcarnitine (C5) | 44664 | 0.82 | 0.0236 | 0.9 | 0.4804 | 0.9 | 0.0581 |
| cyclo(leu-pro) | 37104 | 1.66 | 0.024 | 1.63 | 0.1444 | 1.02 | 0.1666 |
| guanosine | 1573 | 0.45 | 0.0244 | 0.74 | 0.8207 | 0.6 | 0.0056 |
| beta-sitosterol | 27414 | 1.47 | 0.0246 | 1.77 | 0.0052 | 0.83 | 0.4066 |
| oleic ethanolamide | 38102 | 1.22 | 0.0267 | 1.17 | 0.0728 | 1.04 | 0.5567 |
| N-delta-acetylornithine | 43249 | 0.58 | 0.0271 | 0.58 | 0.0268 | 1 | 0.9961 |
| tyrosine | 1299 | 1.2 | 0.0274 | 1.16 | 0.0573 | 1.03 | 0.6583 |
| oleoylcarnitine | 35160 | 1.41 | 0.0275 | 1.47 | 0.006 | 0.96 | 0.2847 |
| leucine | 60 | 0.86 | 0.0279 | 0.89 | 0.0363 | 0.97 | 0.7174 |
| 3beta,7alpha-dihydroxy-5- | 36803 | 1.19 | 0.0284 | 1.26 | 0.0125 | 0.94 | 0.914 |
| cholestenoate | | | | | | | |
| 11-ketoetiocholanolone | 47135 | 0.6 | 0.0287 | 0.71 | 0.2834 | 0.84 | 0.1198 |
| glucuronide | | | | | | | |
| 2- | 34258 | 0.74 | 0.0296 | 0.8 | 0.056 | 0.93 | 0.4729 |
| docosahexaenoylglycerophosphoethanolamine | | | | | | | |
| pyruvate | 22250 | 1.38 | 0.0298 | 1.32 | 0.0749 | 1.04 | 0.5952 |
| azelate (nonanedioate) | 18362 | 0.81 | 0.0304 | 0.87 | 0.8624 | 0.93 | 0.1426 |
| propionylglycine | 31932 | 0.68 | 0.0309 | 0.87 | 0.598 | 0.78 | 0.0032 |
| isobutyrylcarnitine | 33441 | 0.75 | 0.0311 | 0.77 | 0.026 | 0.97 | 0.9435 |
| sebacate (decanedioate) | 32398 | 0.81 | 0.0319 | 0.87 | 0.7508 | 0.93 | 0.1342 |
| tartronate (hydroxymalonate) | 20693 | 0.74 | 0.0321 | 0.76 | 0.0673 | 0.97 | 0.5884 |
| oxalate (ethanedioate) | 20694 | 0.77 | 0.0325 | 0.91 | 0.3104 | 0.85 | 0.0308 |
| 2′-deoxyuridine | 1412 | 0.86 | 0.0337 | 0.97 | 0.7352 | 0.89 | 0.006 |
| lactate | 527 | 1.12 | 0.034 | 1.08 | 0.1581 | 1.04 | 0.2323 |
| orotate | 1505 | 1.73 | 0.0341 | 1.56 | 0.0364 | 1.11 | 0.961 |
| creatinine | 513 | 0.87 | 0.0343 | 0.87 | 0.0389 | 1.01 | 0.9776 |
| 1,2,3-benzenetriol sulfate | 48762 | 0.27 | 0.0347 | 0.65 | 0.7182 | 0.42 | 0.0153 |
| 1,5-anhydroglucitol (1,5-AG) | 20675 | 0.75 | 0.0347 | 0.87 | 0.2161 | 0.86 | 0.0429 |
| choline | 15506 | 0.91 | 0.0351 | 0.89 | 0.015 | 1.02 | 0.5073 |
| isovalerylcarnitine | 34407 | 0.73 | 0.0355 | 0.77 | 0.0371 | 0.95 | 0.9063 |
| AICA ribonucleotide | 38325 | 0.84 | 0.0356 | 0.83 | 0.0482 | 1.02 | 0.9938 |
| beta-alanine | 35838 | 0.81 | 0.036 | 0.86 | 0.2332 | 0.94 | 0.2184 |
| laurylcarnitine | 34534 | 1.41 | 0.036 | 1.25 | 0.097 | 1.13 | 0.6692 |
| 3-hydroxylaurate | 32457 | 1.3 | 0.0366 | 1.1 | 0.516 | 1.18 | 0.0195 |
| citrate | 1564 | 1.23 | 0.0367 | 1.13 | 0.2187 | 1.08 | 0.1638 |
| kynurenate | 1417 | 0.81 | 0.0373 | 0.85 | 0.1148 | 0.95 | 0.4513 |
| cyclo(pro-pro) | 48787 | 0.77 | 0.039 | 0.91 | 0.3159 | 0.85 | 0.0863 |
| serotonin (5HT) | 2342 | 0.71 | 0.0392 | 0.7 | 0.0609 | 1.01 | 0.742 |
| gentisate | 18280 | 0.73 | 0.0394 | 0.76 | 0.3672 | 0.96 | 0.0913 |
| andro steroid monosulfate | 32827 | 1.6 | 0.0396 | 1.58 | 0.0388 | 1.02 | 0.9318 |
| 5alpha-pregnan-3(alpha or | 46172 | 0.22 | 0.0405 | 0.4 | 0.0541 | 0.55 | 0.7487 |
| beta), 20beta-diol disulfate | | | | | | | |
| TL18:3n3 (a-linolenic acid) | 48813 | 0.86 | 0.0408 | 0.9 | 0.1618 | 0.96 | 0.3735 |
| 2-methylmalonyl carnitine | 35482 | 0.83 | 0.0409 | 0.85 | 0.1684 | 0.97 | 0.4287 |
| DSGEGDFXAEGGGVR | 31548 | 1.38 | 0.0416 | 1.12 | 0.142 | 1.24 | 0.5516 |
| linoleate (18:2n6) | 1105 | 1.17 | 0.0418 | 1.15 | 0.0602 | 1.02 | 0.8693 |
| N-acetylalliin | 45404 | 0.57 | 0.0422 | 0.6 | 0.3595 | 0.95 | 0.1056 |
| N4-acetylcytidine | 35130 | 1.26 | 0.0427 | 1.14 | 0.1228 | 1.11 | 0.6173 |
| laurate (12:0) | 1645 | 1.23 | 0.0436 | 0.99 | 0.4022 | 1.24 | 0.1722 |
| 1- | 33822 | 0.79 | 0.0438 | 0.83 | 0.1718 | 0.94 | 0.2527 |
| docosahexaenoylglycerophosphocholine | | | | | | | |
| (22:6n3) | | | | | | | |
| pyroglutamylglycine | 31522 | 1.46 | 0.045 | 1.19 | 0.1144 | 1.22 | 0.6691 |
| tyrosylglutamine | 41459 | 0.71 | 0.046 | 0.8 | 0.248 | 0.89 | 0.2104 |
| deoxycarnitine | 36747 | 0.84 | 0.0468 | 0.86 | 0.1809 | 0.98 | 0.3374 |
| threonylphenylalanine | 31530 | 0.73 | 0.0469 | 1.01 | 0.6211 | 0.72 | 0.0195 |
| creatine | 27718 | 0.81 | 0.0476 | 0.77 | 0.0421 | 1.05 | 0.8569 |
| N-acetylglycine | 27710 | 0.78 | 0.0477 | 0.84 | 0.2125 | 0.93 | 0.3496 |
| 2- | 46203 | 0.67 | 0.0482 | 0.75 | 0.1679 | 0.89 | 0.3325 |
| docosahexaenoylglycerophosphocholine | | | | | | | |
| oleate (18:1n9) | 1359 | 1.25 | 0.0491 | 1.28 | 0.0348 | 0.98 | 0.8262 |
| quinolinate | 1899 | 1.19 | 0.0497 | 1.11 | 0.2899 | 1.07 | 0.2012 |
| ribose | 12083 | 0.74 | 0.0497 | 0.84 | 0.1975 | 0.89 | 0.3929 |

Logistic regression models and area under the curve (AUC) were used to assess how well individual metabolites distinguished the stage 3-4 fibrosis from stage 1-2 and stage 0 fibrosis groups. Logistic regression analysis was performed on the measured values obtained for all 721 named metabolites detected in the samples.

Metabolites with an AUC of >0.620 for distinguishing stage 3-4 fibrosis from stage 1-2 and stage 0 fibrosis patient samples are presented in Table 11.

TABLE 11
AUC of individual metabolites for distinguishing severity of fibrosis

| Biochemical Name | AUC | Biochemical Name | AUC |
|---|---|---|---|
| epiandrosterone sulfate | 0.837 | valerate | 0.663 |
| androsterone sulfate | 0.833 | 4-androsten-3beta,17beta-diol | 0.663 |
| | | disulfate | |
| I-urobilinogen | 0.648 | inosine | 0.662 |
| 16-hydroxypalmitate | 0.66 | 4-hydroxyphenylpyruvate | 0.662 |
| fucose | 0.747 | AICA ribonucleotide | 0.662 |
| taurine | 0.767 | adrenate (22:4n6) | 0.661 |
| 16a-hydroxy DHEA 3-sulfate | 0.724 | orotate | 0.661 |
| dehydroisoandrosterone sulfate | 0.772 | 1- | 0.661 |
| (DHEA-S) | | palmitoylplasmenylemanolamine | |
| 5-methylthioadenosine (MTA) | 0.673 | docosadienoate (22:2n6) | 0.661 |
| gamma-glutamylhistidine | 0.646 | phenylalanylglycine | 0.66 |
| cyclo(L-phe-L-pro) | 0.738 | eicosenoate (20:1n9 or 11) | 0.66 |
| taurocholate | 0.737 | creatinine | 0.658 |
| glycocholate | 0.752 | gamma-glutamylvaline | 0.657 |
| taurochenodeoxycholate | 0.724 | linoleate (18:2n6) | 0.657 |
| glycochenodeoxycholate | 0.755 | phenylacetylcarnitine | 0.657 |
| palmitoleate (16:1n7) | 0.753 | N-delta-acetylornithine | 0.657 |
| TL16:1n7 (palmitoleic acid) | 0.635 | piperine | 0.656 |
| TL16:0 (palmitic acid) | 0.66 | 5-methyluridine (ribothymidine) | 0.656 |
| palmitate (16:0) | 0.663 | glycolithocholate | 0.655 |
| pregnanediol-3-glucuronide | 0.814 | oleic ethanolamide | 0.655 |
| 5alpha-androstan-3beta,17beta-diol | 0.792 | maleate (cis-Butenedioate) | 0.655 |
| monosulfate | | | |
| etiocholanolone glucuronide | 0.785 | serotonin (5HT) | 0.655 |
| 5alpha-androstan-3beta,17beta-diol | 0.783 | pyroglutamylglycine | 0.654 |
| disulfate | | | |
| 5alpha-pregnan-3beta,20alpha-diol | 0.768 | pantothenate | 0.654 |
| monosulfate | | | |
| 4-androsten-3alpha,17alpha-diol | 0.765 | isobutyrylcarnitine | 0.653 |
| monosulfate | | | |
| 4-androsten-3beta,17beta-diol | 0.764 | urate | 0.653 |
| monosulfate | | | |
| 5alpha-androstan-3beta,17alpha-diol | 0.749 | pimelate (heptanedioate) | 0.653 |
| disulfate | | | |
| tryptophylleucine | 0.747 | beta-hydroxyisovalerate | 0.651 |
| 5,6-dihydrothymine | 0.745 | 2-methylmalonyl carnitine | 0.651 |
| 7-alpha-hydroxy-3-oxo-4- | 0.74 | caffeine | 0.65 |
| cholestenoate (7-Hoca) | | | |
| alpha-hydroxyisovalerate | 0.734 | 5alpha-pregnan-3(alpha or | 0.65 |
| | | beta),20beta-diol disulfate | |
| myristoleate (14:1n5) | 0.729 | adenosine | 0.649 |
| valylleucine | 0.727 | oleate (18:1n9) | 0.647 |
| xanthurenate | 0.727 | 1-oleoylplasmenylethanolamine | 0.647 |
| palmitoylcarnitine | 0.722 | 5alpha-androstan-3alpha,17beta- | 0.646 |
| | | diol disulfate | |
| phenylalanylvaline | 0.716 | linolenate [alpha or gamma; | 0.645 |
| | | (18:3n3 or 6)] | |
| sorbitol | 0.714 | glycolithocholate sulfate | 0.644 |
| uridine | 0.713 | 7-methylguanine | 0.644 |
| hypoxanthine | 0.71 | suberate (octanedioate) | 0.643 |
| myristoleoylcarnitine | 0.708 | kynurenate | 0.642 |
| N-oleoyltaurine | 0.708 | creatine | 0.641 |
| N-acetylcarnosine | 0.707 | sulfate | 0.64 |
| oleoylcarnitine | 0.707 | lactate | 0.64 |
| gamma-glutamylleucine | 0.706 | andro steroid monosulfate | 0.639 |
| ergothioneine | 0.704 | leucine | 0.639 |
| glycerol | 0.704 | laurylcarnitine | 0.639 |
| isobutyrylglycine | 0.703 | phenylacetate | 0.638 |
| alpha-ketobutyrate | 0.701 | eicosapentaenoate (EPA; 20:5n3) | 0.637 |
| serine | 0.699 | isovalerylcarnitine | 0.637 |
| tyramine O-sulfate | 0.699 | isovalerate | 0.636 |
| myristoylcarnitine | 0.698 | phosphate | 0.636 |
| isoleucylvaline | 0.698 | N-acetylmethionine | 0.634 |
| 10-heptadecenoate (17:1n7) | 0.698 | TL18:3n6 (g-linolenic acid) | 0.634 |
| glycyltryptophan | 0.696 | 3-methylcrotonylglycine | 0.633 |
| | | (tigloylglycine) | |
| 5-dodecenoate (12:1n7) | 0.694 | acetylcarnitine | 0.633 |
| fumarate | 0.691 | 3beta,7alpha-dihydroxy-5- | 0.632 |
| | | cholestenoate | |
| ADSGEGDFXAEGGGVR | 0.689 | urea | 0.632 |
| 5alpha-androstan-3alpha,17beta-diol | 0.689 | dihomo-linoleate (20:2n6) | 0.63 |
| monosulfate | | | |
| linoleoylcarnitine | 0.688 | 4-methyl-2-oxopentanoate | 0.63 |
| TL20:5n3 (eicosapentaenoic acid) | 0.688 | pyruvate | 0.63 |
| isoleucylglycine | 0.687 | 1- | 0.63 |
| | | margaroylglycerophosphocholine | |
| | | (17:0) | |
| tauro-beta-muricholate | 0.686 | arabinose | 0.629 |
| betaine | 0.685 | palmitoyl ethanolamide | 0.629 |
| nicotinamide | 0.684 | taurodeoxycholate | 0.629 |
| docosatrienoate (22:3n3) | 0.682 | 2′-deoxyuridine | 0.629 |
| thyroxine | 0.682 | glycodeoxycholate | 0.629 |
| isovalerylglycine | 0.682 | 2- | 0.628 |
| | | docosahexaenoylglycerophosphoethanolamine | |
| scyllo-inositol | 0.682 | 10-nonadecenoate (19:1n9) | 0.628 |
| TL18:1n7 (avaccenic acid) | 0.678 | 3-carboxy-4-methyl-5-propyl-2- | 0.627 |
| | | furanpropanoate (CMPF) | |
| pregn steroid monosulfate | 0.676 | 3-methylglutarylcarnitine | 0.627 |
| 1-arachidonoylglycerophosphocholine | 0.675 | alpha-hydroxycaproate | 0.627 |
| (20:4n6) | | | |
| 1- | 0.675 | histidine | 0.627 |
| eicosapentaenoylglycerophosphocholine | | | |
| (20:5n3) | | | |
| cis-vaccenate (18:1n7) | 0.674 | TL24:1n9 (nervonic acid) | 0.626 |
| stigmasterol | 0.673 | indole-3-carboxylic acid | 0.626 |
| N2-methylguanosine | 0.672 | gamma-glutamylalanine | 0.625 |
| xylitol | 0.671 | 1-linolenoylglycerol | 0.624 |
| 4-guanidinobutanoate | 0.67 | 2-arachidonoyl glycerol | 0.624 |
| malate | 0.669 | cyclohexanebutanoic acid | 0.623 |
| valine | 0.668 | 3-hydroxybutyrate (BHBA) | 0.623 |
| 1- | 0.668 | campesterol | 0.623 |
| docosapentaenoylglycerophosphocholine | | | |
| (22:5n3) | | | |
| hydroxybutyrylcarnitine | 0.668 | palmitoyl sphingomyelin | 0.623 |
| glycohyocholate | 0.668 | laurate (12:0) | 0.622 |
| myristate (14:0) | 0.667 | tyrosylglutamine | 0.622 |
| N-palmitoyl glycine | 0.667 | sebacate (decanedioate) | 0.622 |
| alliin | 0.665 | gamma-glutamyltyrosine | 0.621 |
| pyridoxate | 0.665 | 1- | 0.621 |
| | | docosahexaenoylglycerophosphocholine | |
| | | (22:6n3) | |
| choline | 0.664 | biliverdin | 0.621 |
| N1-Methyl-2-pyridone-5-carboxamide | 0.664 | arginine | 0.62 |

### Example 4

### Metabolite Biomarkers of Fibrosis in Human Serum

In another example, serum samples from 200 subjects spanning the spectrum of nonalcoholic fatty liver disease were analyzed. Patient samples were classified into five groups according to disease severity based on the fibrosis stage (stage 0, no fibrosis (N=12); stage 1, mild severity (N=38); stage 2, moderate severity (N=100); stage 3, high severity (N=42); stage 4, cirrhosis (N=8)). All samples were analyzed using four global metabolic profiling mass spectrometry platforms, as well as the GC-FID analysis for fatty acids, cholesterol metabolism lipids, and Vitamin E. A total of 790 named metabolites and 361 unnamed metabolites were detected in the sample cohort. Clinical parameters including Age, Gender, Race, Ethnicity, Height/Weight/Body mass index (BMI), Smoking history, Diabetes history, Steatosis, Fibrosis, Lobular Inflammation, Portal Inflammation, Hepatocellular ballooning, NAFLD Activity Score (NAS), Fasting glucose, Fasting insulin, Aspartate aminotransferase (AST), Alanine aminotransferase (ALT), Alkaline phosphatase, Total cholesterol, High-density lipoprotein cholesterol (HDL), Low-density lipoprotein cholesterol (LDL), Triglycerides, HBA1c, and Hemoglobin (HGB) were provided for the subjects.

The statistical significance and predictive performance of metabolites detected in the samples, used individually or in combinations, to stage fibrosis in these subjects was assessed using t-tests, AUC calculations, logistic regression and random forest analysis. For comparison, the performance of Age, Type 2 Diabetes, BMI, HDL Cholesterol, Gender, Fructose, and Past Alcohol Use, which are commonly measured clinical parameters, was also evaluated individually and in combinations. The results of these analyses are presented in this example. These results show that many metabolites alone have an AUC higher than obtained using clinical parameters alone, and in some cases, outperformed combinations of clinical parameters. Further, our analyses identified combinations of metabolites that had better predictive performance than any of the combinations of clinical parameters.

The measured levels of the 1151 metabolites detected in the samples were analyzed using Welch's two-sample t-tests to compare the levels measured in samples collected from subjects with more severe fibrosis to the levels measured in samples collected from subjects with less severe fibrosis or no fibrosis. Metabolites detected in the study are presented in Table 12. Comparisons presented in Table 12 are Stage 2-4 vs. Stage 0-1, Stage 3-4 vs. Stage 1-2, Stage 3-4 vs. Stage 0-1, Stage 4 vs. Stage 0, Stage 3-4 vs. Stage 0, and Stage 1-2 vs. Stage 0, Stage 3-4 vs. Stage 1-2, Stage 3-4 vs. Stage 2, and Stage 2 vs. Stage 0-1. Table 12 includes, for each metabolite, the biochemical name of the metabolite, the internal identifier for the biomarker compound in the in-house chemical library of authentic standards (ComplD), the fold change (FC) of the biomarker for each comparison, which is the ratio of the mean level of that biomarker in one sample type as compared to the mean level in a different sample type, and the p-value determined in the statistical analysis of the data concerning the biomarkers. Fold change values in bold font indicate that the p-value for the given comparison was less than 0.05.

TABLE 12
Biomarkers of fibrosis and the stage of fibrosis

| | STAGE 2-4 | | STAGE 3-4 | | STAGE 3-4 | | STAGE 4 | | STAGE 3-4 | | STAGE 1-2 | | STAGE 3-4 | | STAGE 3-4 | | STAGE 2 | |
|---|---|---|---|---|---|---|---|---|---|---|---|---|---|---|---|---|---|---|
| | STAGE 0-1 | | STAGE 0-2 | | STAGE 0-1 | | STAGE 0 | | STAGE 0 | | STAGE 0 | | STAGE 1-2 | | STAGE 2 | | STAGE 0-1 | |
| Biochemical Name | FC | p-value | FC | p-value | FC | p-value | FC | p-value | FC | p-value | FC | p-value | FC | p-value | FC | p-value | FC | p-value |
| glutarate | 1.45 | 0.001 | 1.39 | 0.003 | 1.7 | p < .001 | 1.7 | 0.058 | 2.01 | 0.005 | 1.49 | 0.071 | 1.35 | 0.007 | 1.27 | 0.044 | 1.33 | 0.011 |
| (pentanedioate) | | | | | | | | | | | | | | | | | | |
| epiandrosterone sulfate | 0.67 | 0.025 | 0.63 | 0.034 | 0.52 | 0.007 | 0.2 | 0.014 | 0.7 | 0.080 | 1.12 | 0.691 | 0.63 | 0.040 | 0.71 | 0.122 | 0.74 | 0.124 |
| androsterone sulfate | 0.73 | 0.037 | 0.62 | 0.039 | 0.55 | 0.010 | 0.2 | 0.005 | 0.66 | 0.104 | 1.07 | 0.690 | 0.62 | 0.047 | 0.67 | 0.134 | 0.82 | 0.162 |
| I-urobilinogen | 2 | p < .001 | 1.73 | p < .001 | 2.56 | <.0001 | 4.26 | 0.022 | 5.87 | 0.001 | 3.6 | 0.056 | 1.63 | p < .001 | 1.49 | 0.006 | 1.72 | 0.004 |
| 16-hydroxypalmitate | 1 | 0.787 | 1.19 | 0.002 | 1.13 | 0.045 | 1.24 | 0.056 | 1.27 | 0.013 | 1.07 | 0.525 | 1.18 | 0.002 | 1.22 | 0.001 | 0.93 | 0.420 |
| fucose | 1.23 | 0.019 | 1.27 | 0.001 | 1.39 | p < .001 | 1.37 | 0.037 | 1.2 | 0.204 | 0.94 | 0.103 | 1.27 | p < .001 | 1.21 | 0.005 | 1.15 | 0.231 |
| taurine | 0.92 | 0.049 | 0.84 | 0.008 | 0.82 | 0.004 | 0.91 | 0.396 | 0.86 | 0.032 | 1.03 | 0.763 | 0.84 | 0.010 | 0.85 | 0.029 | 0.97 | 0.313 |
| 3-hydroxydecanoate | 1.33 | 0.006 | 1.29 | 0.018 | 1.5 | 0.002 | 1.4 | 0.112 | 1.6 | 0.007 | 1.25 | 0.187 | 1.27 | 0.026 | 1.21 | 0.087 | 1.24 | 0.055 |
| 3-hydroxyoctanoate | 1.59 | p < .001 | 1.32 | 0.034 | 1.77 | p < .001 | 1.69 | 0.069 | 1.96 | 0.001 | 1.52 | 0.013 | 1.29 | 0.059 | 1.18 | 0.254 | 1.5 | 0.001 |
| X-11871 | 0.69 | 0.001 | 0.71 | 0.004 | 0.59 | p < .001 | 0.34 | 0.014 | 0.56 | 0.003 | 0.76 | 0.120 | 0.73 | 0.006 | 0.8 | 0.027 | 0.74 | 0.022 |
| X-12850 | 2.15 | p < .001 | 1.66 | p < .001 | 2.65 | <.0001 | 3.98 | 0.013 | 4.33 | p < .001 | 2.75 | 0.007 | 1.57 | 0.002 | 1.4 | 0.019 | 1.9 | 0.002 |
| 16a-hydroxy DHEA 3- | 1.69 | 0.005 | 1.21 | 0.014 | 1.74 | 0.001 | 2.39 | 0.027 | 2.44 | 0.005 | 2.11 | 0.028 | 1.16 | 0.041 | 1.05 | 0.124 | 1.67 | 0.032 |
| sulfate | | | | | | | | | | | | | | | | | | |
| dehydroisoandrosterone | 0.88 | 0.101 | 0.86 | 0.120 | 0.82 | 0.052 | 0.39 | 0.058 | 1.1 | 0.984 | 1.3 | 0.312 | 0.85 | 0.095 | 0.89 | 0.246 | 0.91 | 0.272 |
| sulfate (DHEA-S) | | | | | | | | | | | | | | | | | | |
| 2-aminoheptanoate | 1.24 | 0.001 | 1.18 | 0.165 | 1.34 | 0.009 | 1.08 | 0.753 | 1.21 | 0.302 | 1.02 | 0.716 | 1.18 | 0.182 | 1.12 | 0.611 | 1.2 | 0.003 |
| X-19561 | 1.63 | p < .001 | 1.41 | 0.090 | 1.88 | 0.001 | 1.41 | 0.465 | 1.8 | 0.050 | 1.3 | 0.239 | 1.38 | 0.120 | 1.25 | 0.485 | 1.5 | p < .001 |
| gamma- | 1.06 | 0.148 | 1.54 | 0.061 | 1.42 | 0.047 | 1.47 | 0.550 | 1.8 | 0.132 | 1.18 | 0.678 | 1.52 | 0.070 | 1.61 | 0.120 | 0.88 | 0.387 |
| glutamylhistidine | | | | | | | | | | | | | | | | | | |
| cyclo(L-phe-L-pro) | 1.81 | 0.007 | 0.98 | 0.320 | 1.58 | 0.020 | 1.45 | 0.530 | 1.85 | 0.068 | 1.96 | 0.129 | 0.94 | 0.443 | 0.82 | 0.920 | 1.92 | 0.013 |
| taurocholate | 2.3 | 0.008 | 3.13 | p < .001 | 4.04 | <.0002 | 2.64 | 0.103 | 2.82 | 0.037 | 0.89 | 0.577 | 3.16 | p < .001 | 2.82 | 0.001 | 1.43 | 0.155 |
| glycocholate | 1.85 | 0.002 | 2.08 | p < .001 | 2.67 | p < .001 | 3.13 | 0.099 | 3.1 | 0.010 | 1.54 | 0.235 | 2.02 | 0.001 | 1.87 | 0.004 | 1.43 | 0.039 |
| taurochenodeoxycholate | 2.43 | 0.004 | 3.53 | p < .001 | 4.48 | <.0001 | 2.43 | 0.068 | 3.52 | 0.039 | 1 | 0.449 | 3.53 | p < .001 | 3.19 | 0.001 | 1.4 | 0.096 |
| glycochenodeoxycholate | 2.06 | 0.001 | 2.31 | 0.001 | 3.12 | p < .001 | 2.62 | 0.027 | 4.16 | 0.004 | 1.87 | 0.108 | 2.23 | 0.002 | 2.05 | 0.011 | 1.53 | 0.026 |
| isoleucine | 1.08 | 0.012 | 1.06 | 0.075 | 1.11 | 0.009 | 0.98 | 0.912 | 1.1 | 0.194 | 1.04 | 0.446 | 1.05 | 0.105 | 1.03 | 0.347 | 1.07 | 0.033 |
| glutamate | 1.24 | 0.001 | 1.13 | 0.165 | 1.3 | 0.005 | 1.45 | 0.261 | 1.38 | 0.037 | 1.23 | 0.094 | 1.12 | 0.255 | 1.06 | 0.667 | 1.22 | 0.002 |
| alpha-ketoglutarate | 1.27 | 0.008 | 1.15 | 0.074 | 1.33 | 0.005 | 1.34 | 0.211 | 1.3 | 0.111 | 1.14 | 0.457 | 1.13 | 0.097 | 1.07 | 0.351 | 1.24 | 0.032 |
| 1- | 0.83 | p < .001 | 0.92 | 0.077 | 0.82 | 0.001 | 0.8 | 0.148 | 0.81 | 0.019 | 0.87 | 0.099 | 0.93 | 0.117 | 0.97 | 0.449 | 0.84 | p < .001 |
| stearoylglycerophosphoinositol | | | | | | | | | | | | | | | | | | |
| fumarate | 1.3 | p < .001 | 1.15 | 0.044 | 1.36 | p < .001 | 1.61 | 0.024 | 1.25 | 0.087 | 1.09 | 0.670 | 1.14 | 0.052 | 1.07 | 0.387 | 1.28 | 0.001 |
| malate | 1.31 | p < .001 | 1.13 | 0.017 | 1.34 | p < .001 | 1.73 | 0.008 | 1.47 | 0.001 | 1.32 | 0.016 | 1.11 | 0.037 | 1.04 | 0.214 | 1.29 | 0.001 |
| X-12263 | 5.36 | <.0001 | 1.33 | 0.018 | 5.24 | <.0001 | 6.62 | 0.153 | 6.88 | 0.001 | 5.54 | 0.027 | 1.24 | 0.033 | 0.97 | 0.223 | 5.42 | p < .001 |
| X-14662 | 5.38 | <.0001 | 0.54 | 0.003 | 2.6 | <.0001 | 5.95 | 0.051 | 5.29 | 0.003 | 10.7 | 0.029 | 0.5 | 0.011 | 0.38 | 0.156 | 6.77 | 0.001 |
| X-15497 | 1.44 | p < .001 | 1.34 | 0.059 | 1.64 | 0.001 | 1.21 | 0.655 | 1.48 | 0.091 | 1.12 | 0.548 | 1.32 | 0.070 | 1.22 | 0.352 | 1.34 | 0.001 |
| N-methylproline | 0.53 | p < .001 | 0.86 | 0.334 | 0.58 | 0.006 | 0.48 | 0.065 | 0.57 | 0.015 | 0.64 | 0.025 | 0.9 | 0.502 | 1.14 | 0.871 | 0.51 | p < .001 |
| ribose | 1.59 | p < .001 | 1.13 | 0.357 | 1.58 | 0.006 | 2.3 | 0.122 | 1.54 | 0.123 | 1.39 | 0.203 | 1.11 | 0.473 | 0.99 | 0.780 | 1.59 | 0.001 |
| gamma- | 1.21 | 0.001 | 1.11 | 0.030 | 1.25 | 0.001 | 1.18 | 0.184 | 1.21 | 0.099 | 1.1 | 0.347 | 1.1 | 0.048 | 1.05 | 0.388 | 1.19 | 0.002 |
| glutamylisoleucine | | | | | | | | | | | | | | | | | | |
| X-11537 | 0.72 | 0.001 | 0.76 | 0.005 | 0.63 | p < .001 | 0.38 | 0.018 | 0.64 | 0.010 | 0.83 | 0.248 | 0.77 | 0.007 | 0.84 | 0.033 | 0.76 | 0.015 |
| X-17453 | 2.11 | 0.001 | 1.32 | 0.001 | 2.25 | p < .001 | 4.32 | 0.042 | 5.91 | 0.004 | 4.77 | 0.114 | 1.24 | 0.003 | 1.1 | 0.029 | 2.05 | 0.016 |
| 1-dihomo- | 0.84 | 0.001 | 1.04 | 0.961 | 0.91 | 0.066 | 0.71 | 0.078 | 0.91 | 0.078 | 0.87 | 0.028 | 1.05 | 0.891 | 1.12 | 0.414 | 0.81 | 0.001 |
| linolenylglycerol | | | | | | | | | | | | | | | | | | |
| (alpha, gamma) | | | | | | | | | | | | | | | | | | |
| maleate (cis- | 1.62 | 0.001 | 0.88 | 0.261 | 1.34 | 0.004 | 1.3 | 0.104 | 1.42 | 0.024 | 1.66 | 0.073 | 0.86 | 0.373 | 0.76 | 0.913 | 1.77 | 0.006 |
| Butenedioate) | | | | | | | | | | | | | | | | | | |
| 1-dihomo- | 0.76 | 0.001 | 0.95 | 0.428 | 0.78 | 0.016 | 0.6 | 0.028 | 0.81 | 0.048 | 0.84 | 0.073 | 0.96 | 0.560 | 1.05 | 0.833 | 0.74 | 0.002 |
| linoleoylglycerophosphocholine | | | | | | | | | | | | | | | | | | |
| (20:2n6) | | | | | | | | | | | | | | | | | | |
| 2- | 0.76 | 0.002 | 0.87 | 0.082 | 0.74 | 0.004 | 0.71 | 0.111 | 0.75 | 0.002 | 0.85 | 0.012 | 0.89 | 0.125 | 0.96 | 0.352 | 0.77 | 0.007 |
| stearoylglycerophosphoinositol | | | | | | | | | | | | | | | | | | |
| X-14302 | 1.59 | 0.002 | 1.48 | 0.006 | 1.91 | p < .001 | 1.4 | 0.490 | 1.51 | 0.250 | 1.02 | 0.644 | 1.48 | 0.006 | 1.33 | 0.076 | 1.44 | 0.015 |
| 2-arachidonoylglycerol | 0.84 | 0.003 | 0.9 | 0.091 | 0.81 | 0.012 | 0.67 | 0.043 | 0.81 | 0.017 | 0.9 | 0.120 | 0.91 | 0.114 | 0.95 | 0.238 | 0.86 | 0.023 |
| (20:4) | | | | | | | | | | | | | | | | | | |
| gamma-glutamylvaline | 1.13 | 0.003 | 1.08 | 0.075 | 1.16 | 0.004 | 1.09 | 0.486 | 1.14 | 0.090 | 1.06 | 0.399 | 1.07 | 0.099 | 1.04 | 0.399 | 1.11 | 0.010 |
| X-14658 | 2.64 | 0.003 | 2.4 | 0.002 | 3.97 | p < .001 | 3.13 | 0.036 | 3.84 | 0.007 | 1.65 | 0.090 | 2.32 | 0.004 | 2 | 0.019 | 1.98 | 0.042 |
| dihydroferulic acid | 3.28 | 0.003 | 1.72 | 0.041 | 3.95 | 0.003 | 3.03 | 0.056 | 6.01 | 0.004 | 3.72 | 0.055 | 1.62 | 0.066 | 1.34 | 0.179 | 2.95 | 0.028 |
| phenylalanylserine | 0.71 | 0.004 | 0.92 | 0.473 | 0.73 | 0.034 | 0.31 | 0.042 | 0.49 | 0.002 | 0.5 | 0.001 | 0.99 | 0.707 | 1.05 | 0.929 | 0.69 | 0.007 |
| 2-hydroxydecanoate | 1.37 | 0.004 | 1.37 | 0.022 | 1.59 | 0.002 | 1.37 | 0.209 | 1.75 | 0.015 | 1.31 | 0.152 | 1.34 | 0.036 | 1.27 | 0.104 | 1.25 | 0.031 |
| threonate | 0.79 | 0.004 | 0.83 | 0.026 | 0.73 | 0.003 | 0.87 | 0.802 | 0.82 | 0.510 | 0.99 | 0.732 | 0.83 | 0.022 | 0.89 | 0.117 | 0.83 | 0.030 |
| X-21892 | 1.24 | 0.004 | 1.15 | 0.057 | 1.31 | 0.004 | 1.42 | 0.062 | 1.57 | p < .001 | 1.39 | 0.004 | 1.13 | 0.109 | 1.09 | 0.269 | 1.2 | 0.023 |
| X-18922 | 1.36 | 0.006 | 1.22 | 0.101 | 1.47 | 0.007 | 1.2 | 0.512 | 1.46 | 0.168 | 1.21 | 0.529 | 1.21 | 0.125 | 1.13 | 0.450 | 1.31 | 0.017 |
| X-12739 | 1.34 | 0.006 | 1.05 | 0.141 | 1.3 | 0.009 | 2.29 | 0.004 | 2.01 | 0.005 | 1.98 | 0.018 | 1.01 | 0.256 | 0.96 | 0.630 | 1.36 | 0.013 |
| oxalate (ethanedioate) | 0.79 | 0.006 | 0.82 | 0.028 | 0.72 | 0.003 | 0.85 | 0.834 | 0.84 | 0.602 | 1.04 | 0.647 | 0.81 | 0.023 | 0.87 | 0.124 | 0.82 | 0.041 |
| 2-hydroxy-3- | 1.33 | 0.006 | 1.3 | 0.024 | 1.5 | 0.003 | 1.17 | 0.407 | 1.67 | 0.012 | 1.31 | 0.113 | 1.28 | 0.040 | 1.22 | 0.101 | 1.24 | 0.055 |
| methylvalerate | | | | | | | | | | | | | | | | | | |
| alpha-glutamyltyrosine | 0.62 | 0.008 | 0.68 | 0.055 | 0.53 | 0.005 | 0.32 | 0.095 | 0.44 | 0.028 | 0.61 | 0.143 | 0.72 | 0.088 | 0.8 | 0.243 | 0.67 | 0.033 |
| xylonate | 1.36 | 0.008 | 1.27 | 0.029 | 1.52 | 0.003 | 1.5 | 0.016 | 1.49 | 0.112 | 1.19 | 0.634 | 1.26 | 0.037 | 1.18 | 0.170 | 1.29 | 0.037 |
| sphingomyelin | 0.88 | 0.008 | 0.95 | 0.388 | 0.87 | 0.035 | 0.83 | 0.260 | 0.8 | 0.047 | 0.82 | 0.071 | 0.97 | 0.552 | 1 | 0.942 | 0.88 | 0.015 |
| (d18:1/20:1, | | | | | | | | | | | | | | | | | | |
| d18:2/20:0) | | | | | | | | | | | | | | | | | | |
| 1- | 0.86 | 0.008 | 0.83 | 0.041 | 0.78 | 0.007 | 0.79 | 0.180 | 0.86 | 0.043 | 1.03 | 0.586 | 0.83 | 0.047 | 0.87 | 0.117 | 0.9 | 0.083 |
| pentadecanoylglycerol | | | | | | | | | | | | | | | | | | |
| (15:0) | | | | | | | | | | | | | | | | | | |
| 1- | 0.88 | 0.010 | 0.94 | 0.156 | 0.87 | 0.017 | 0.69 | 0.033 | 0.79 | 0.020 | 0.83 | 0.052 | 0.96 | 0.260 | 0.98 | 0.520 | 0.88 | 0.021 |
| arachidonoylglycerophosphoinositol | | | | | | | | | | | | | | | | | | |
| 1-linoleoylglycerol | 0.79 | 0.010 | 0.97 | 0.713 | 0.82 | 0.070 | 0.87 | 0.278 | 0.92 | 0.047 | 0.94 | 0.028 | 0.98 | 0.842 | 1.07 | 0.633 | 0.77 | 0.010 |
| (18:2) | | | | | | | | | | | | | | | | | | |
| dimethylglycine | 1.19 | 0.010 | 1.11 | 0.046 | 1.23 | 0.006 | 1.26 | 0.087 | 1.16 | 0.245 | 1.05 | 0.985 | 1.11 | 0.050 | 1.06 | 0.289 | 1.17 | 0.027 |
| glycoursodeoxycholate | 2.31 | 0.010 | 0.78 | 0.093 | 1.64 | 0.008 | 1.55 | 0.218 | 2.05 | 0.052 | 2.76 | 0.159 | 0.74 | 0.158 | 0.62 | 0.474 | 2.65 | 0.026 |
| caproate (6:0) | 1.15 | 0.010 | 0.94 | 0.459 | 1.06 | 0.234 | 1.02 | 0.790 | 1.04 | 0.631 | 1.12 | 0.315 | 0.93 | 0.389 | 0.89 | 0.105 | 1.19 | 0.004 |
| 1- | 0.8 | 0.011 | 0.92 | 0.228 | 0.8 | 0.028 | 0.5 | 0.020 | 0.74 | 0.035 | 0.78 | 0.089 | 0.94 | 0.318 | 1 | 0.596 | 0.79 | 0.025 |
| linolenoylglycerophosphocholine | | | | | | | | | | | | | | | | | | |
| (18:3n3) | | | | | | | | | | | | | | | | | | |
| cyclo(leu-pro) | 1.45 | 0.011 | 1.3 | 0.015 | 1.62 | 0.002 | 1.6 | 0.133 | 1.7 | 0.046 | 1.33 | 0.355 | 1.28 | 0.022 | 1.19 | 0.096 | 1.37 | 0.062 |
| tauro-beta-muricholate | 5.04 | 0.011 | 2.59 | 0.108 | 7.47 | 0.019 | 14.2 | 0.061 | 7.39 | 0.084 | 3.01 | 0.426 | 2.45 | 0.128 | 1.95 | 0.261 | 3.83 | 0.070 |
| 1- | 0.88 | 0.012 | 0.96 | 0.287 | 0.88 | 0.038 | 0.69 | 0.052 | 0.88 | 0.203 | 0.91 | 0.401 | 0.97 | 0.343 | 1 | 0.697 | 0.88 | 0.023 |
| linoleoylglycerophosphocholine | | | | | | | | | | | | | | | | | | |
| (18:2n6) | | | | | | | | | | | | | | | | | | |
| 1- | 0.84 | 0.012 | 0.98 | 0.401 | 0.87 | 0.054 | 0.55 | 0.008 | 0.83 | 0.059 | 0.84 | 0.090 | 0.99 | 0.525 | 1.04 | 0.899 | 0.83 | 0.017 |
| eicosatrienoylglycerophosphocholine | | | | | | | | | | | | | | | | | | |
| (20:3) | | | | | | | | | | | | | | | | | | |
| 1-arachidonylglycerol | 0.82 | 0.012 | 0.96 | 0.646 | 0.84 | 0.057 | 0.59 | 0.002 | 0.74 | 0.004 | 0.75 | 0.004 | 0.98 | 0.932 | 1.03 | 0.670 | 0.81 | 0.014 |
| (20:4) | | | | | | | | | | | | | | | | | | |
| pelargonate (9:0) | 1.08 | 0.013 | 1.04 | 0.419 | 1.09 | 0.058 | 1.16 | 0.162 | 1.16 | 0.015 | 1.12 | 0.017 | 1.03 | 0.566 | 1.02 | 0.924 | 1.08 | 0.016 |
| arginine | 0.9 | 0.013 | 0.96 | 0.417 | 0.9 | 0.050 | 0.89 | 0.408 | 0.92 | 0.409 | 0.95 | 0.620 | 0.97 | 0.465 | 1 | 0.933 | 0.9 | 0.021 |
| N-acetylneuraminate | 1.23 | 0.013 | 1.17 | 0.053 | 1.31 | 0.006 | 1.49 | 0.052 | 1.29 | 0.092 | 1.12 | 0.609 | 1.16 | 0.064 | 1.11 | 0.199 | 1.18 | 0.077 |
| sphingomyelin | 0.88 | 0.013 | 1 | 0.903 | 0.91 | 0.146 | 0.78 | 0.092 | 0.82 | 0.043 | 0.81 | 0.020 | 1.02 | 0.644 | 1.06 | 0.317 | 0.86 | 0.007 |
| (d18:1/18:1, | | | | | | | | | | | | | | | | | | |
| d18:2/18:0) | | | | | | | | | | | | | | | | | | |
| homoarginine | 0.71 | 0.014 | 0.85 | 0.431 | 0.69 | 0.043 | 0.64 | 0.346 | 0.72 | 0.293 | 0.84 | 0.432 | 0.86 | 0.515 | 0.95 | 0.954 | 0.72 | 0.021 |
| palmitoyl-palmitoyl- | 1.19 | 0.014 | 1.28 | p < .001 | 1.37 | p < .001 | 1.31 | 0.238 | 1.42 | 0.013 | 1.12 | 0.448 | 1.27 | p < .001 | 1.24 | 0.002 | 1.1 | 0.180 |
| glycerophosphocholine | | | | | | | | | | | | | | | | | | |
| 2-hydroxystearate | 0.92 | 0.015 | 1.03 | 0.556 | 0.96 | 0.284 | 0.92 | 0.245 | 1.01 | 0.976 | 0.99 | 0.702 | 1.03 | 0.531 | 1.06 | 0.157 | 0.9 | 0.005 |
| alpha- | 1.36 | 0.015 | 1.35 | 0.019 | 1.58 | 0.004 | 1.27 | 0.533 | 1.72 | 0.016 | 1.3 | 0.236 | 1.33 | 0.028 | 1.26 | 0.075 | 1.25 | 0.118 |
| hydroxyisovalerate | | | | | | | | | | | | | | | | | | |
| tyrosylglutamine | 0.85 | 0.015 | 0.87 | 0.089 | 0.8 | 0.015 | 0.59 | 0.067 | 0.84 | 0.023 | 0.96 | 0.184 | 0.88 | 0.115 | 0.91 | 0.252 | 0.88 | 0.070 |
| 1- | 0.78 | 0.016 | 0.82 | 0.033 | 0.72 | 0.007 | 0.62 | 0.111 | 0.9 | 0.110 | 1.11 | 0.929 | 0.81 | 0.035 | 0.88 | 0.098 | 0.81 | 0.114 |
| arachidoylglycerophosphocholine | | | | | | | | | | | | | | | | | | |
| (20:0) | | | | | | | | | | | | | | | | | | |
| 2- | 0.76 | 0.017 | 0.87 | 0.653 | 0.74 | 0.231 | 0.79 | 0.323 | 0.76 | 0.053 | 0.86 | 0.009 | 0.88 | 0.480 | 0.96 | 0.216 | 0.77 | 0.009 |
| docosahexaenoylglcyerol | | | | | | | | | | | | | | | | | | |
| inosine | 1.84 | 0.017 | 1.63 | 0.332 | 2.29 | 0.047 | 4.42 | 0.420 | 2.18 | 0.311 | 1.37 | 0.535 | 1.59 | 0.382 | 1.42 | 0.804 | 1.61 | 0.027 |
| X-17145 | 1.32 | 0.018 | 0.86 | 0.706 | 1.11 | 0.086 | 1.07 | 0.843 | 2.1 | 0.046 | 2.56 | 0.041 | 0.82 | 0.977 | 0.78 | 0.637 | 1.42 | 0.018 |
| glycerol 3-phosphate | 4.49 | 0.019 | 1.3 | 0.825 | 4.37 | 0.295 | 0.73 | 0.938 | 3.42 | 0.626 | 2.77 | 0.426 | 1.23 | 0.766 | 0.96 | 0.445 | 4.54 | 0.017 |
| (G3P) | | | | | | | | | | | | | | | | | | |
| tartronate | 0.85 | 0.019 | 0.78 | 0.011 | 0.73 | 0.003 | 1.12 | 0.793 | 0.86 | 0.479 | 1.12 | 0.576 | 0.77 | 0.009 | 0.8 | 0.050 | 0.91 | 0.141 |
| (hydroxymalonate) | | | | | | | | | | | | | | | | | | |
| xylitol | 1.48 | 0.019 | 1.16 | 0.019 | 1.52 | 0.003 | 1.29 | 0.104 | 1.35 | 0.250 | 1.18 | 0.812 | 1.15 | 0.019 | 1.04 | 0.091 | 1.46 | 0.131 |
| xylose | 1.98 | 0.021 | 1.14 | 0.414 | 1.9 | 0.570 | 2.06 | 0.089 | 2.39 | 0.106 | 2.2 | 0.006 | 1.09 | 0.289 | 0.94 | 0.160 | 2.02 | 0.006 |
| pyroglutamylglutamine | 1.26 | 0.021 | 1.12 | 0.359 | 1.29 | 0.046 | 1.13 | 0.613 | 1.18 | 0.381 | 1.06 | 0.529 | 1.11 | 0.437 | 1.05 | 0.999 | 1.24 | 0.025 |
| ursodeoxycholate | 3.1 | 0.022 | 0.66 | 0.152 | 1.87 | 0.021 | 1.63 | 0.369 | 1.83 | 0.160 | 2.91 | 0.321 | 0.63 | 0.220 | 0.5 | 0.526 | 3.72 | 0.047 |
| X-21659 | 1.18 | 0.022 | 1.17 | 0.093 | 1.28 | 0.017 | 0.62 | 0.836 | 1.1 | 0.205 | 0.94 | 0.393 | 1.18 | 0.140 | 1.13 | 0.418 | 1.13 | 0.045 |
| N6-acetyllysine | 1.09 | 0.023 | 1.05 | 0.214 | 1.1 | 0.033 | 1.04 | 0.842 | 1.05 | 0.518 | 1 | 0.869 | 1.05 | 0.214 | 1.02 | 0.583 | 1.08 | 0.050 |
| 1- | 0.85 | 0.023 | 0.85 | 0.027 | 0.78 | 0.006 | 0.73 | 0.158 | 0.82 | 0.179 | 0.96 | 0.785 | 0.85 | 0.031 | 0.89 | 0.111 | 0.88 | 0.108 |
| pentadecanoylglycerophosphocholine | | | | | | | | | | | | | | | | | | |
| (15:0) | | | | | | | | | | | | | | | | | | |
| ribulose | 1.53 | 0.024 | 1.24 | 0.868 | 1.64 | 0.162 | 7.36 | 0.019 | 2.42 | 0.022 | 2.03 | 0.010 | 1.19 | 0.901 | 1.11 | 0.613 | 1.48 | 0.021 |
| 3-(4- | 1.13 | 0.025 | 1.12 | 0.077 | 1.19 | 0.016 | 1.22 | 0.296 | 1.28 | 0.048 | 1.16 | 0.169 | 1.1 | 0.122 | 1.08 | 0.237 | 1.1 | 0.088 |
| hydroxyphenyl)lactate | | | | | | | | | | | | | | | | | | |
| arabinose | 1.25 | 0.026 | 1.08 | 0.373 | 1.26 | 0.055 | 1.4 | 0.374 | 1.19 | 0.659 | 1.1 | 0.844 | 1.07 | 0.371 | 1.01 | 0.900 | 1.24 | 0.038 |
| S- | 1.3 | 0.026 | 0.95 | 0.742 | 1.17 | 0.257 | 1.5 | 0.138 | 1.21 | 0.380 | 1.3 | 0.229 | 0.93 | 0.618 | 0.87 | 0.283 | 1.36 | 0.015 |
| adenosylhomocysteine | | | | | | | | | | | | | | | | | | |
| (SAH) | | | | | | | | | | | | | | | | | | |
| 2-linoleoylglycerol | 0.82 | 0.026 | 0.98 | 0.394 | 0.85 | 0.095 | 0.92 | 0.472 | 1 | 0.195 | 1.03 | 0.370 | 0.98 | 0.429 | 1.06 | 0.732 | 0.81 | 0.041 |
| (18:2) | | | | | | | | | | | | | | | | | | |
| tauroursodeoxycholate | 2.77 | 0.026 | 1.43 | 0.063 | 3 | 0.013 | 1.28 | 0.760 | 1.7 | 0.245 | 1.2 | 0.782 | 1.41 | 0.070 | 1.13 | 0.191 | 2.65 | 0.111 |
| 3-methyl-2-oxovalerate | 1.1 | 0.027 | 1.07 | 0.081 | 1.13 | 0.015 | 1 | 0.910 | 1.19 | 0.065 | 1.12 | 0.206 | 1.07 | 0.128 | 1.05 | 0.285 | 1.08 | 0.083 |
| gamma-CEHC | 0.86 | 0.027 | 0.91 | 0.159 | 0.84 | 0.029 | 0.93 | 0.893 | 0.8 | 0.298 | 0.88 | 0.644 | 0.92 | 0.185 | 0.95 | 0.423 | 0.88 | 0.075 |
| lactate | 1.09 | 0.028 | 1.04 | 0.392 | 1.11 | 0.069 | 1.23 | 0.299 | 1.16 | 0.111 | 1.12 | 0.168 | 1.03 | 0.522 | 1.01 | 0.833 | 1.09 | 0.044 |
| N-acetylphenylalanine | 1.27 | 0.028 | 1.12 | 0.091 | 1.31 | 0.015 | 1.6 | 0.056 | 1.51 | 0.022 | 1.38 | 0.084 | 1.1 | 0.156 | 1.05 | 0.291 | 1.25 | 0.101 |
| imidazole propionate | 1.46 | 0.028 | 1.71 | 0.006 | 1.96 | 0.003 | 0.84 | 0.816 | 1.67 | 0.226 | 0.97 | 0.737 | 1.72 | 0.006 | 1.61 | 0.022 | 1.21 | 0.202 |
| 3-ureidopropionate | 1.19 | 0.028 | 1.09 | 0.137 | 1.22 | 0.023 | 1.76 | 0.001 | 1.76 | 0.001 | 1.66 | 0.003 | 1.06 | 0.336 | 1.04 | 0.459 | 1.17 | 0.063 |
| 1- | 0.77 | 0.028 | 0.91 | 0.577 | 0.77 | 0.090 | 0.77 | 0.315 | 0.76 | 0.104 | 0.82 | 0.126 | 0.93 | 0.721 | 1 | 0.865 | 0.77 | 0.034 |
| margaroylglycerophosphocholine | | | | | | | | | | | | | | | | | | |
| (17:0) | | | | | | | | | | | | | | | | | | |
| alpha-hydroxycaproate | 1.18 | 0.028 | 1.14 | 0.331 | 1.25 | 0.076 | 1.16 | 0.950 | 1.31 | 0.080 | 1.16 | 0.160 | 1.13 | 0.390 | 1.09 | 0.651 | 1.14 | 0.053 |
| 1-linolenoylglycerol | 0.85 | 0.029 | 0.97 | 0.705 | 0.87 | 0.107 | 0.78 | 0.107 | 0.85 | 0.016 | 0.86 | 0.010 | 0.98 | 0.875 | 1.03 | 0.769 | 0.84 | 0.040 |
| (18:3) | | | | | | | | | | | | | | | | | | |
| gamma- | 1.12 | 0.030 | 0.99 | 0.768 | 1.08 | 0.237 | 1.15 | 0.402 | 1.14 | 0.277 | 1.17 | 0.188 | 0.98 | 0.557 | 0.95 | 0.260 | 1.14 | 0.015 |
| glutamyltyrosine | | | | | | | | | | | | | | | | | | |
| 3-methylglutaconate | 0.89 | 0.030 | 0.9 | 0.271 | 0.84 | 0.045 | 0.82 | 0.398 | 0.69 | 0.010 | 0.75 | 0.022 | 0.92 | 0.424 | 0.93 | 0.632 | 0.91 | 0.070 |
| phenyllactate (PLA) | 1.17 | 0.033 | 1.22 | 0.093 | 1.31 | 0.025 | 1.27 | 0.340 | 1.33 | 0.110 | 1.1 | 0.351 | 1.21 | 0.122 | 1.18 | 0.224 | 1.11 | 0.120 |
| cysteine-glutathione | 1.1 | 0.033 | 0.47 | 0.005 | 0.58 | 0.002 | 0.57 | 0.527 | 0.58 | 0.122 | 1.27 | 0.792 | 0.46 | 0.006 | 0.43 | 0.024 | 1.36 | 0.227 |
| disulfide | | | | | | | | | | | | | | | | | | |
| tyrosine | 1.07 | 0.034 | 1 | 0.792 | 1.05 | 0.301 | 1.04 | 0.658 | 1.1 | 0.259 | 1.1 | 0.154 | 0.99 | 0.619 | 0.97 | 0.336 | 1.08 | 0.016 |
| 1- | 0.87 | 0.034 | 1.03 | 0.989 | 0.92 | 0.221 | 0.68 | 0.033 | 0.87 | 0.109 | 0.83 | 0.057 | 1.05 | 0.835 | 1.1 | 0.501 | 0.84 | 0.022 |
| oleoylglycerophosphocholine | | | | | | | | | | | | | | | | | | |
| (18:1) | | | | | | | | | | | | | | | | | | |
| N-acetylmethionine | 0.84 | 0.035 | 0.69 | 0.018 | 0.66 | 0.006 | 0.66 | 0.106 | 0.59 | 0.002 | 0.84 | 0.038 | 0.7 | 0.034 | 0.71 | 0.069 | 0.93 | 0.170 |
| N-acetyltyrosine | 1.21 | 0.035 | 1.07 | 0.446 | 1.22 | 0.083 | 1.48 | 0.102 | 1.35 | 0.093 | 1.29 | 0.130 | 1.05 | 0.595 | 1.01 | 0.903 | 1.21 | 0.053 |
| coprostanol | 1.59 | 0.035 | 1.24 | 0.742 | 1.69 | 0.148 | 0.92 | 0.724 | 1.2 | 0.606 | 0.97 | 0.366 | 1.24 | 0.675 | 1.09 | 0.703 | 1.55 | 0.030 |
| X-21474 | 1.05 | 0.036 | 1.17 | 0.255 | 1.17 | 0.051 | 0.56 | 0.508 | 0.86 | 0.301 | 0.71 | 0.434 | 1.21 | 0.344 | 1.18 | 0.762 | 0.99 | 0.047 |
| TL24:0 (lignoceric | 0.69 | 0.036 | 0.87 | 0.158 | 0.7 | 0.035 | 0.41 | 0.005 | 0.63 | 0.060 | 0.69 | 0.123 | 0.91 | 0.273 | 1 | 0.560 | 0.69 | 0.056 |
| acid) | | | | | | | | | | | | | | | | | | |
| 2- | 0.87 | 0.036 | 1 | 0.727 | 0.9 | 0.153 | 0.76 | 0.180 | 0.96 | 0.584 | 0.95 | 0.673 | 1 | 0.771 | 1.06 | 0.768 | 0.85 | 0.036 |
| linoleoylglycerophosphocholine | | | | | | | | | | | | | | | | | | |
| 1- | 0.81 | 0.037 | 0.9 | 0.146 | 0.79 | 0.033 | 1.08 | 0.869 | 0.9 | 0.671 | 1.01 | 0.806 | 0.9 | 0.137 | 0.96 | 0.413 | 0.82 | 0.081 |
| arachidonoylglyercophosphate | | | | | | | | | | | | | | | | | | |
| adenosine | 0.76 | 0.037 | 0.74 | 0.091 | 0.65 | 0.021 | 0.48 | 0.270 | 0.49 | 0.108 | 0.64 | 0.278 | 0.78 | 0.138 | 0.8 | 0.277 | 0.81 | 0.111 |
| pyroglutamine | 1.31 | 0.038 | 0.87 | 0.208 | 1.11 | 0.624 | 0.94 | 0.901 | 1.08 | 0.948 | 1.26 | 0.432 | 0.85 | 0.181 | 0.78 | 0.044 | 1.41 | 0.009 |
| 2- | 0.81 | 0.038 | 0.95 | 0.768 | 0.83 | 0.135 | 0.7 | 0.112 | 0.84 | 0.174 | 0.87 | 0.169 | 0.97 | 0.911 | 1.03 | 0.652 | 0.81 | 0.037 |
| stearoylglycerophosphocholine | | | | | | | | | | | | | | | | | | |
| tyramine O-sulfate | 1.53 | 0.038 | 1.32 | 0.327 | 1.71 | 0.069 | 1.56 | 0.550 | 1.44 | 0.692 | 1.1 | 0.829 | 1.31 | 0.319 | 1.18 | 0.690 | 1.44 | 0.070 |
| N-acetylkynurenine | 2.24 | 0.039 | 3.45 | 0.116 | 4.14 | 0.031 | 1.46 | 0.103 | 4.04 | 0.085 | 1.19 | 0.214 | 3.41 | 0.175 | 3.19 | 0.310 | 1.3 | 0.097 |
| stachydrine | 0.65 | 0.039 | 0.82 | 0.904 | 0.64 | 0.155 | 0.64 | 0.496 | 0.52 | 0.283 | 0.6 | 0.254 | 0.87 | 0.919 | 0.97 | 0.501 | 0.66 | 0.033 |
| beta-hydroxyisovalerate | 1.18 | 0.040 | 1.17 | 0.270 | 1.27 | 0.068 | 1.06 | 0.707 | 1.4 | 0.059 | 1.22 | 0.121 | 1.16 | 0.364 | 1.13 | 0.561 | 1.13 | 0.075 |
| alpha- | 1.16 | 0.040 | 1.23 | 0.071 | 1.31 | 0.024 | 1.38 | 0.158 | 1.61 | 0.008 | 1.33 | 0.028 | 1.21 | 0.141 | 1.2 | 0.171 | 1.09 | 0.141 |
| hydroxyisocaproate | | | | | | | | | | | | | | | | | | |
| N-acetylglutamine | 1.3 | 0.041 | 1.17 | 0.102 | 1.37 | 0.023 | 1.75 | 0.035 | 1.52 | 0.042 | 1.33 | 0.211 | 1.14 | 0.139 | 1.09 | 0.268 | 1.27 | 0.147 |
| 3-hydroxy-3- | 1.15 | 0.041 | 1.02 | 0.569 | 1.13 | 0.107 | 1.18 | 0.112 | 1.09 | 0.517 | 1.07 | 0.727 | 1.02 | 0.603 | 0.98 | 0.971 | 1.16 | 0.063 |
| methylglutarate | | | | | | | | | | | | | | | | | | |
| 2- | 0.89 | 0.042 | 1.01 | 0.695 | 0.93 | 0.166 | 0.69 | 0.063 | 0.94 | 0.427 | 0.92 | 0.486 | 1.02 | 0.767 | 1.06 | 0.831 | 0.88 | 0.041 |
| eicosatrienoylglycerophosphocholine | | | | | | | | | | | | | | | | | | |
| ribitol | 1.11 | 0.046 | 1.09 | 0.010 | 1.16 | 0.004 | 1.15 | 0.105 | 1.14 | 0.083 | 1.05 | 0.998 | 1.09 | 0.012 | 1.06 | 0.060 | 1.09 | 0.193 |
| 1- | 0.87 | 0.046 | 0.99 | 0.758 | 0.9 | 0.177 | 0.87 | 0.476 | 0.93 | 0.407 | 0.93 | 0.438 | 1 | 0.833 | 1.04 | 0.779 | 0.86 | 0.050 |
| palmitoylglycerophosphoinositol | | | | | | | | | | | | | | | | | | |
| levulinate (4- | 1.22 | 0.047 | 1.11 | 0.336 | 1.26 | 0.077 | 1.08 | 0.566 | 1.33 | 0.080 | 1.22 | 0.156 | 1.09 | 0.432 | 1.05 | 0.631 | 1.2 | 0.118 |
| oxovalerate) | | | | | | | | | | | | | | | | | | |
| gamma- | 1.09 | 0.047 | 1.02 | 0.840 | 1.08 | 0.195 | 1.14 | 0.349 | 1.08 | 0.409 | 1.07 | 0.400 | 1.01 | 0.931 | 0.99 | 0.666 | 1.09 | 0.043 |
| glutamylphenylalanine | | | | | | | | | | | | | | | | | | |
| oleamide | 0.88 | 0.048 | 1.03 | 0.956 | 0.93 | 0.240 | 1.5 | 0.486 | 1.02 | 0.236 | 1 | 0.138 | 1.03 | 0.946 | 1.08 | 0.596 | 0.86 | 0.042 |
| glucose | 1.08 | 0.048 | 1.13 | 0.028 | 1.16 | 0.013 | 1.14 | 0.224 | 1.19 | 0.042 | 1.06 | 0.436 | 1.13 | 0.035 | 1.12 | 0.066 | 1.04 | 0.244 |
| pyroglutamylvaline | 1.16 | 0.048 | 1.15 | 0.160 | 1.24 | 0.044 | 1.19 | 0.568 | 1.15 | 0.521 | 1 | 0.701 | 1.15 | 0.154 | 1.11 | 0.359 | 1.12 | 0.128 |
| homocitrulline | 1.27 | 0.048 | 1.11 | 0.254 | 1.29 | 0.055 | 1.31 | 0.077 | 1.22 | 0.531 | 1.11 | 0.892 | 1.1 | 0.255 | 1.03 | 0.562 | 1.25 | 0.110 |
| propyl 4- | 0.85 | 0.049 | 0.69 | 0.178 | 0.67 | 0.041 | 2.32 | 0.708 | 1.3 | 0.376 | 1.96 | 0.834 | 0.66 | 0.193 | 0.7 | 0.446 | 0.95 | 0.114 |
| hydroxybenzoate | | | | | | | | | | | | | | | | | | |
| sulfate | | | | | | | | | | | | | | | | | | |
| 1,5-anhydroglucitol | 0.87 | 0.049 | 0.71 | 0.001 | 0.69 | 0.001 | 0.67 | 0.083 | 0.69 | 0.001 | 0.96 | 0.254 | 0.71 | 0.002 | 0.72 | 0.005 | 0.96 | 0.406 |
| (1,5-AG) | | | | | | | | | | | | | | | | | | |
| beta-sitosterol | 0.91 | 0.049 | 1.01 | 0.835 | 0.94 | 0.304 | 0.95 | 0.824 | 0.95 | 0.458 | 0.93 | 0.307 | 1.02 | 0.743 | 1.06 | 0.417 | 0.89 | 0.037 |
| 3- | 1.22 | 0.051 | 1.04 | 0.293 | 1.2 | 0.061 | 1.32 | 0.133 | 1.44 | 0.071 | 1.42 | 0.147 | 1.01 | 0.403 | 0.97 | 0.691 | 1.23 | 0.090 |
| methylglutarylcarnitine | | | | | | | | | | | | | | | | | | |
| 7-methylxanthine | 0.84 | 0.051 | 1.17 | 0.084 | 0.99 | 0.029 | 0.63 | 0.293 | 0.88 | 0.005 | 0.73 | 0.035 | 1.2 | 0.126 | 1.29 | 0.198 | 0.77 | 0.165 |
| 3,7-dimethylurate | 0.74 | 0.052 | 0.93 | 0.064 | 0.76 | 0.022 | 0.58 | 0.261 | 0.7 | 0.003 | 0.74 | 0.029 | 0.95 | 0.109 | 1.04 | 0.188 | 0.73 | 0.153 |
| N-acetylcitrulline | 1.7 | 0.052 | 1.52 | 0.063 | 2.05 | 0.019 | 3.11 | 0.318 | 2.08 | 0.152 | 1.4 | 0.680 | 1.49 | 0.074 | 1.35 | 0.172 | 1.52 | 0.193 |
| 1- | 0.88 | 0.052 | 0.94 | 0.237 | 0.87 | 0.058 | 0.56 | 0.001 | 0.78 | 0.016 | 0.82 | 0.038 | 0.96 | 0.362 | 0.98 | 0.562 | 0.89 | 0.096 |
| arachidonoylglycerophosphocholine | | | | | | | | | | | | | | | | | | |
| (20:4n6) | | | | | | | | | | | | | | | | | | |
| guanidinoacetate | 0.91 | 0.052 | 0.93 | 0.228 | 0.88 | 0.060 | 0.89 | 0.642 | 0.99 | 0.727 | 1.07 | 0.723 | 0.93 | 0.213 | 0.96 | 0.474 | 0.92 | 0.124 |
| 5-oxoproline | 1.12 | 0.053 | 1.06 | 0.662 | 1.14 | 0.169 | 1.77 | 0.053 | 1.33 | 0.029 | 1.28 | 0.024 | 1.04 | 0.906 | 1.03 | 0.876 | 1.11 | 0.054 |
| TL22:0 (behenic acid) | 0.8 | 0.053 | 0.91 | 0.324 | 0.79 | 0.072 | 0.72 | 0.034 | 0.9 | 0.211 | 1 | 0.435 | 0.91 | 0.382 | 0.98 | 0.817 | 0.81 | 0.072 |
| 2-hydroxyglutarate | 1.22 | 0.054 | 1.06 | 0.049 | 1.21 | 0.013 | 1.42 | 0.023 | 1.4 | 0.016 | 1.35 | 0.095 | 1.03 | 0.090 | 0.99 | 0.197 | 1.22 | 0.174 |
| 1- | 0.87 | 0.054 | 0.94 | 0.319 | 0.86 | 0.080 | 0.62 | 0.054 | 0.79 | 0.119 | 0.82 | 0.216 | 0.96 | 0.416 | 0.99 | 0.673 | 0.87 | 0.091 |
| docosapentaenoylglycerophosphocholine | | | | | | | | | | | | | | | | | | |
| (22:5n3) | | | | | | | | | | | | | | | | | | |
| orotate | 1.45 | 0.055 | 1.88 | 0.113 | 2.07 | 0.045 | 1.41 | 0.055 | 3.26 | 0.002 | 1.8 | 0.007 | 1.82 | 0.169 | 1.8 | 0.222 | 1.14 | 0.166 |
| urate | 1.1 | 0.055 | 0.98 | 0.881 | 1.06 | 0.231 | 1.04 | 0.634 | 1.1 | 0.300 | 1.14 | 0.230 | 0.97 | 0.693 | 0.94 | 0.358 | 1.12 | 0.041 |
| sorbitol | 1.27 | 0.056 | 0.99 | 0.898 | 1.19 | 0.219 | 1.2 | 0.542 | 1.21 | 0.542 | 1.25 | 0.523 | 0.97 | 0.949 | 0.91 | 0.644 | 1.31 | 0.055 |
| kynurenine | 1.1 | 0.056 | 1.02 | 0.687 | 1.09 | 0.160 | 1.19 | 0.182 | 1.14 | 0.185 | 1.13 | 0.195 | 1.01 | 0.829 | 0.99 | 0.826 | 1.1 | 0.061 |
| 5-hydroxyindoleacetate | 1.13 | 0.058 | 1 | 0.711 | 1.09 | 0.142 | 0.95 | 0.965 | 0.89 | 0.473 | 0.89 | 0.320 | 1.01 | 0.599 | 0.95 | 0.723 | 1.14 | 0.061 |
| creatine | 0.89 | 0.058 | 0.99 | 0.858 | 0.92 | 0.216 | 0.95 | 0.968 | 0.96 | 0.756 | 0.96 | 0.796 | 1 | 0.890 | 1.04 | 0.667 | 0.88 | 0.053 |
| O-methylcatechol | 0.8 | 0.059 | 0.92 | 0.103 | 0.79 | 0.035 | 1.21 | 0.761 | 1.02 | 0.063 | 1.12 | 0.378 | 0.91 | 0.125 | 0.99 | 0.249 | 0.8 | 0.162 |
| sulfate | | | | | | | | | | | | | | | | | | |
| 1- | 0.86 | 0.059 | 0.97 | 0.860 | 0.87 | 0.202 | 0.8 | 0.230 | 0.85 | 0.228 | 0.87 | 0.199 | 0.98 | 0.997 | 1.02 | 0.632 | 0.85 | 0.053 |
| docosahexaenoylglycerophosphocholine | | | | | | | | | | | | | | | | | | |
| (22:6n3) | | | | | | | | | | | | | | | | | | |
| TL18:0 (stearic acid) | 0.95 | 0.060 | 0.98 | 0.545 | 0.95 | 0.122 | 0.9 | 0.114 | 0.95 | 0.277 | 0.96 | 0.362 | 0.99 | 0.647 | 1 | 0.921 | 0.95 | 0.067 |
| N-acetylisoleucine | 1.28 | 0.060 | 1.11 | 0.201 | 1.3 | 0.056 | 1.41 | 0.049 | 1.4 | 0.179 | 1.28 | 0.389 | 1.09 | 0.256 | 1.03 | 0.470 | 1.26 | 0.122 |
| 1- | 0.86 | 0.061 | 0.98 | 0.785 | 0.88 | 0.186 | 0.74 | 0.102 | 0.91 | 0.230 | 0.92 | 0.220 | 0.98 | 0.899 | 1.04 | 0.699 | 0.85 | 0.057 |
| stearoylglycerophosphocholine | | | | | | | | | | | | | | | | | | |
| (18:0) | | | | | | | | | | | | | | | | | | |
| gamma-glutamylleucine | 1.08 | 0.061 | 1.02 | 0.715 | 1.08 | 0.180 | 1.02 | 0.712 | 1.09 | 0.320 | 1.07 | 0.337 | 1.02 | 0.853 | 1 | 0.797 | 1.08 | 0.060 |
| dopamine sulfate | 1.24 | 0.062 | 1.05 | 0.241 | 1.23 | 0.060 | 0.88 | 0.981 | 0.96 | 0.815 | 0.9 | 0.660 | 1.06 | 0.223 | 0.98 | 0.571 | 1.25 | 0.115 |
| mannose | 1.11 | 0.063 | 1.15 | 0.010 | 1.2 | 0.006 | 1.23 | 0.074 | 1.31 | 0.007 | 1.15 | 0.140 | 1.14 | 0.017 | 1.13 | 0.032 | 1.06 | 0.323 |
| 1- | 0.91 | 0.065 | 1.01 | 0.992 | 0.94 | 0.277 | 0.81 | 0.141 | 0.93 | 0.295 | 0.91 | 0.214 | 1.02 | 0.889 | 1.05 | 0.550 | 0.89 | 0.049 |
| palmitoylglycerophosphocholine | | | | | | | | | | | | | | | | | | |
| (16:0) | | | | | | | | | | | | | | | | | | |
| chiro-inositol | 0.55 | 0.067 | 0.88 | 0.798 | 0.6 | 0.191 | 0.74 | 0.655 | 0.45 | 0.118 | 0.47 | 0.102 | 0.96 | 0.949 | 1.14 | 0.634 | 0.52 | 0.056 |
| gluconate | 1.15 | 0.069 | 1.18 | 0.121 | 1.26 | 0.044 | 1.87 | 0.067 | 1.28 | 0.161 | 1.08 | 0.673 | 1.18 | 0.135 | 1.15 | 0.262 | 1.09 | 0.191 |
| X-12812 | 0.5 | 0.069 | 0.62 | 0.009 | 0.43 | 0.008 | 0.78 | 0.335 | 0.34 | 0.429 | 0.5 | 0.842 | 0.67 | 0.008 | 0.81 | 0.037 | 0.53 | 0.256 |
| alanine | 1.07 | 0.069 | 1.01 | 0.711 | 1.06 | 0.185 | 0.96 | 0.745 | 1.04 | 0.557 | 1.03 | 0.625 | 1.01 | 0.778 | 0.99 | 0.820 | 1.07 | 0.073 |
| andro steroid | 1.52 | 0.071 | 1.17 | 0.265 | 1.56 | 0.075 | 1.87 | 0.061 | 2.08 | 0.026 | 1.84 | 0.041 | 1.13 | 0.486 | 1.04 | 0.583 | 1.49 | 0.125 |
| monosulfate | | | | | | | | | | | | | | | | | | |
| X-12063 | 0.9 | 0.071 | 0.83 | 0.301 | 0.8 | 0.085 | 0.57 | 0.137 | 0.71 | 0.200 | 0.84 | 0.418 | 0.84 | 0.355 | 0.85 | 0.570 | 0.95 | 0.156 |
| mannitol | 3.16 | 0.072 | 2.01 | 0.370 | 4.19 | 0.117 | 1.56 | 0.949 | 3.76 | 0.318 | 1.95 | 0.575 | 1.93 | 0.410 | 1.59 | 0.658 | 2.64 | 0.126 |
| caprylate (8:0) | 1.14 | 0.077 | 0.95 | 0.883 | 1.06 | 0.286 | 1.17 | 0.307 | 1.21 | 0.070 | 1.3 | 0.035 | 0.93 | 0.669 | 0.9 | 0.428 | 1.17 | 0.062 |
| citrate | 1.11 | 0.077 | 1.09 | 0.081 | 1.15 | 0.029 | 1.53 | 0.014 | 1.24 | 0.052 | 1.16 | 0.196 | 1.07 | 0.124 | 1.06 | 0.222 | 1.09 | 0.213 |
| arachidonate (20:4n6) | 0.95 | 0.078 | 1 | 0.835 | 0.96 | 0.265 | 0.8 | 0.045 | 0.9 | 0.019 | 0.89 | 0.006 | 1.01 | 0.965 | 1.02 | 0.765 | 0.94 | 0.074 |
| 5alpha-androstan- | 0.7 | 0.078 | 0.64 | 0.011 | 0.54 | 0.009 | 0.2 | 0.031 | 0.7 | 0.483 | 1.11 | 0.538 | 0.63 | 0.009 | 0.69 | 0.037 | 0.78 | 0.315 |
| 3beta,17beta-diol | | | | | | | | | | | | | | | | | | |
| monosulfate | | | | | | | | | | | | | | | | | | |
| ethylmalonate | 1.24 | 0.079 | 1.18 | 0.192 | 1.33 | 0.067 | 1.09 | 0.707 | 1.17 | 0.625 | 0.99 | 0.747 | 1.18 | 0.183 | 1.12 | 0.383 | 1.19 | 0.187 |
| 4-androsten- | 0.91 | 0.079 | 1 | 0.144 | 0.93 | 0.053 | 0.25 | 0.026 | 1.11 | 0.953 | 1.12 | 0.403 | 0.99 | 0.117 | 1.03 | 0.294 | 0.9 | 0.216 |
| 3beta,17beta-diol | | | | | | | | | | | | | | | | | | |
| monosulfate | | | | | | | | | | | | | | | | | | |
| asparagine | 1.06 | 0.079 | 1.02 | 0.755 | 1.06 | 0.230 | 1.19 | 0.030 | 1.14 | 0.073 | 1.13 | 0.060 | 1.01 | 0.981 | 1 | 0.819 | 1.06 | 0.077 |
| 2-hydroxypalmitate | 0.94 | 0.080 | 1.06 | 0.183 | 1 | 0.855 | 0.97 | 0.643 | 1.04 | 0.812 | 0.97 | 0.355 | 1.06 | 0.162 | 1.1 | 0.048 | 0.91 | 0.019 |
| N-acetylleucine | 1.13 | 0.080 | 1.08 | 0.270 | 1.16 | 0.082 | 1.31 | 0.093 | 1.22 | 0.122 | 1.14 | 0.276 | 1.07 | 0.339 | 1.04 | 0.546 | 1.11 | 0.156 |
| tetradecanedioate | 1.12 | 0.082 | 0.97 | 0.425 | 1.06 | 0.108 | 1.34 | 0.442 | 1.38 | 0.057 | 1.46 | 0.107 | 0.94 | 0.547 | 0.93 | 0.897 | 1.15 | 0.112 |
| TL22:5n6 (osbond acid) | 1.13 | 0.083 | 1.06 | 0.347 | 1.14 | 0.100 | 1.27 | 0.288 | 1.35 | 0.052 | 1.3 | 0.080 | 1.04 | 0.527 | 1.02 | 0.715 | 1.12 | 0.128 |
| gamma- | 0.94 | 0.083 | 0.93 | 0.289 | 0.9 | 0.084 | 0.95 | 0.558 | 0.94 | 0.515 | 1.01 | 0.923 | 0.93 | 0.301 | 0.95 | 0.565 | 0.95 | 0.172 |
| glutamylglutamine | | | | | | | | | | | | | | | | | | |
| 3-hydroxy-2- | 1.1 | 0.084 | 1.12 | 0.214 | 1.17 | 0.076 | 0.97 | 0.812 | 1.13 | 0.313 | 1.02 | 0.592 | 1.12 | 0.250 | 1.09 | 0.452 | 1.07 | 0.157 |
| ethylpropionate | | | | | | | | | | | | | | | | | | |
| methyl glucopyranoside | 0.55 | 0.086 | 0.77 | 0.284 | 0.54 | 0.098 | 0.74 | 0.183 | 0.71 | 0.056 | 0.92 | 0.127 | 0.77 | 0.368 | 0.97 | 0.567 | 0.56 | 0.139 |
| (alpha + beta) | | | | | | | | | | | | | | | | | | |
| methionine sulfoxide | 1.12 | 0.086 | 1.03 | 0.837 | 1.11 | 0.259 | 1.46 | 0.054 | 1.26 | 0.083 | 1.25 | 0.055 | 1.01 | 0.986 | 0.99 | 0.731 | 1.12 | 0.078 |
| 2-piperidinone | 1.28 | 0.086 | 1.45 | 0.007 | 1.58 | 0.006 | 1.19 | 0.350 | 1.59 | 0.077 | 1.11 | 0.446 | 1.44 | 0.010 | 1.39 | 0.024 | 1.13 | 0.381 |
| campesterol | 0.91 | 0.086 | 0.99 | 0.668 | 0.92 | 0.406 | 0.99 | 0.900 | 0.97 | 0.828 | 0.98 | 0.614 | 0.99 | 0.623 | 1.03 | 0.319 | 0.9 | 0.062 |
| 5alpha-androstan- | 1.77 | 0.088 | 1.3 | 0.488 | 1.9 | 0.149 | 2.5 | 0.411 | 2.48 | 0.159 | 1.99 | 0.224 | 1.24 | 0.598 | 1.12 | 0.851 | 1.7 | 0.124 |
| 3alpha,17alpha-diol | | | | | | | | | | | | | | | | | | |
| disulfate | | | | | | | | | | | | | | | | | | |
| N-acetyl-1- | 1.25 | 0.088 | 1.18 | 0.092 | 1.34 | 0.034 | 1.77 | 0.028 | 1.65 | 0.039 | 1.43 | 0.156 | 1.16 | 0.142 | 1.12 | 0.233 | 1.2 | 0.247 |
| methylhistidine | | | | | | | | | | | | | | | | | | |
| 5alpha-androstan- | 0.72 | 0.089 | 0.57 | 0.027 | 0.51 | 0.017 | 0.12 | 0.003 | 0.65 | 0.147 | 1.14 | 0.841 | 0.57 | 0.031 | 0.61 | 0.075 | 0.83 | 0.317 |
| 3alpha,17beta-diol | | | | | | | | | | | | | | | | | | |
| monosulfate | | | | | | | | | | | | | | | | | | |
| 1- | 0.92 | 0.090 | 0.92 | 0.175 | 0.88 | 0.062 | 0.92 | 0.515 | 0.94 | 0.357 | 1.03 | 0.945 | 0.92 | 0.184 | 0.94 | 0.372 | 0.94 | 0.206 |
| linoleoylglycerophosphoinositol | | | | | | | | | | | | | | | | | | |
| 7-methylurate | 0.68 | 0.091 | 0.65 | 0.010 | 0.54 | 0.010 | 0.43 | 0.364 | 0.34 | 0.002 | 0.49 | 0.035 | 0.7 | 0.022 | 0.72 | 0.030 | 0.75 | 0.366 |
| 5-acetylamino-6-amino- | 0.76 | 0.092 | 0.9 | 0.088 | 0.76 | 0.040 | 0.4 | 0.202 | 0.66 | 0.155 | 0.71 | 0.468 | 0.93 | 0.113 | 1 | 0.207 | 0.76 | 0.240 |
| 3-methyluracil | | | | | | | | | | | | | | | | | | |
| gamma-tocopherol | 1.18 | 0.095 | 1.33 | 0.001 | 1.39 | 0.003 | 1.2 | 0.632 | 1.48 | 0.056 | 1.13 | 0.521 | 1.32 | 0.002 | 1.3 | 0.005 | 1.08 | 0.452 |
| 5alpha-pregnan- | 1.06 | 0.095 | 0.78 | 0.283 | 0.86 | 0.088 | 1.08 | 0.306 | 1.09 | 0.196 | 1.43 | 0.447 | 0.76 | 0.333 | 0.74 | 0.566 | 1.15 | 0.183 |
| 3beta,20alpha-diol | | | | | | | | | | | | | | | | | | |
| monosulfate | | | | | | | | | | | | | | | | | | |
| 1- | 1.27 | 0.096 | 1.02 | 0.333 | 1.22 | 0.100 | 2.1 | 0.056 | 1.44 | 0.109 | 1.44 | 0.193 | 1 | 0.451 | 0.95 | 0.699 | 1.29 | 0.150 |
| methylimidazoleacetate | | | | | | | | | | | | | | | | | | |
| 1- | 0.79 | 0.096 | 0.78 | 0.215 | 0.69 | 0.072 | 0.56 | 0.154 | 0.61 | 0.148 | 0.77 | 0.318 | 0.79 | 0.286 | 0.83 | 0.485 | 0.83 | 0.183 |
| eicosapentaenoylglycerophosphocholine | | | | | | | | | | | | | | | | | | |
| (20:5n3) | | | | | | | | | | | | | | | | | | |
| scyllo-inositol | 0.83 | 0.097 | 0.93 | 0.405 | 0.82 | 0.132 | 1.11 | 0.956 | 1.08 | 0.269 | 1.18 | 0.488 | 0.91 | 0.455 | 0.99 | 0.747 | 0.83 | 0.144 |
| 2- | 0.88 | 0.099 | 0.97 | 0.581 | 0.89 | 0.188 | 0.72 | 0.091 | 0.88 | 0.303 | 0.9 | 0.383 | 0.98 | 0.670 | 1.02 | 0.982 | 0.88 | 0.115 |
| palmitoylglycerophosphocholine | | | | | | | | | | | | | | | | | | |
| aspartate | 1.15 | 0.100 | 1.04 | 0.940 | 1.15 | 0.338 | 1.39 | 0.418 | 1.31 | 0.129 | 1.28 | 0.079 | 1.02 | 0.887 | 0.99 | 0.679 | 1.16 | 0.083 |
| N6- | 1.11 | 0.101 | 1.06 | 0.228 | 1.12 | 0.079 | 1.06 | 0.371 | 1.14 | 0.403 | 1.09 | 0.895 | 1.05 | 0.244 | 1.02 | 0.543 | 1.1 | 0.168 |
| carbamoylthreonyladenosine | | | | | | | | | | | | | | | | | | |
| ascorbate (Vitamin C) | 0.52 | 0.101 | 0.8 | 0.851 | 0.54 | 0.260 | 0.59 | 0.340 | 0.89 | 0.499 | 1.12 | 0.502 | 0.79 | 0.927 | 1.05 | 0.693 | 0.52 | 0.093 |
| serine | 1.07 | 0.104 | 1.06 | 0.215 | 1.1 | 0.086 | 1.35 | 0.009 | 1.22 | 0.026 | 1.17 | 0.056 | 1.05 | 0.345 | 1.04 | 0.456 | 1.05 | 0.183 |
| sphingomyelin | 0.85 | 0.104 | 0.88 | 0.107 | 0.8 | 0.052 | 0.71 | 0.267 | 0.74 | 0.173 | 0.83 | 0.448 | 0.89 | 0.139 | 0.92 | 0.242 | 0.87 | 0.239 |
| (d18:2/14:0, | | | | | | | | | | | | | | | | | | |
| d18:1/14:1) | | | | | | | | | | | | | | | | | | |
| alpha-CEHC | 0.95 | 0.107 | 0.5 | 0.081 | 0.55 | 0.038 | 1.42 | 0.594 | 0.4 | 0.073 | 0.78 | 0.200 | 0.51 | 0.138 | 0.48 | 0.234 | 1.15 | 0.248 |
| glucuronide | | | | | | | | | | | | | | | | | | |
| glycocholenate sulfate | 1.18 | 0.111 | 1.08 | 0.425 | 1.2 | 0.143 | 1.75 | 0.102 | 1.32 | 0.094 | 1.24 | 0.154 | 1.06 | 0.534 | 1.02 | 0.723 | 1.17 | 0.187 |
| 2-oleoylglycerol (18:1) | 0.87 | 0.115 | 1.03 | 0.977 | 0.92 | 0.346 | 0.77 | 0.289 | 1.02 | 0.554 | 0.99 | 0.469 | 1.03 | 0.910 | 1.09 | 0.575 | 0.85 | 0.097 |
| caprate (10:0) | 1.11 | 0.118 | 0.98 | 0.984 | 1.07 | 0.327 | 1.27 | 0.355 | 1.45 | 0.025 | 1.52 | 0.008 | 0.96 | 0.810 | 0.94 | 0.577 | 1.13 | 0.096 |
| trans-4-hydroxyproline | 1.17 | 0.120 | 1.15 | 0.145 | 1.25 | 0.067 | 1.25 | 0.638 | 1.11 | 0.570 | 0.97 | 0.859 | 1.15 | 0.141 | 1.1 | 0.293 | 1.13 | 0.275 |
| 1-palmitoylglycerol (1- | 0.87 | 0.122 | 1.07 | 0.397 | 0.95 | 0.690 | 0.94 | 0.574 | 1.12 | 0.799 | 1.05 | 0.639 | 1.06 | 0.382 | 1.14 | 0.165 | 0.83 | 0.055 |
| monopalmitin) | | | | | | | | | | | | | | | | | | |
| 3-methylxanthine | 0.75 | 0.125 | 0.88 | 0.108 | 0.74 | 0.058 | 0.49 | 0.352 | 0.42 | 0.024 | 0.43 | 0.083 | 0.98 | 0.175 | 0.98 | 0.216 | 0.75 | 0.310 |
| N6-methyladenosine | 1.18 | 0.125 | 1.11 | 0.080 | 1.23 | 0.034 | 0.92 | 0.759 | 1.13 | 0.405 | 1.02 | 0.944 | 1.11 | 0.082 | 1.06 | 0.182 | 1.16 | 0.386 |
| hexadecanedioate | 1.01 | 0.125 | 1.03 | 0.160 | 1.03 | 0.076 | 1.38 | 0.230 | 1.53 | 0.010 | 1.53 | 0.042 | 1 | 0.255 | 1.03 | 0.402 | 1 | 0.214 |
| hydroxybutyrylcarnitine | 1.2 | 0.127 | 1.26 | 0.029 | 1.36 | 0.025 | 1.31 | 0.126 | 1.68 | 0.110 | 1.37 | 0.498 | 1.23 | 0.040 | 1.22 | 0.080 | 1.12 | 0.359 |
| hypoxanthine | 1.36 | 0.127 | 1.18 | 0.380 | 1.44 | 0.848 | 3.73 | 0.142 | 1.89 | 0.292 | 1.65 | 0.053 | 1.15 | 0.275 | 1.09 | 0.169 | 1.33 | 0.035 |
| piperine | 0.96 | 0.128 | 1.18 | 0.140 | 1.09 | 0.080 | 0.66 | 0.775 | 1.04 | 0.204 | 0.88 | 0.331 | 1.19 | 0.223 | 1.22 | 0.360 | 0.89 | 0.204 |
| 1- | 0.83 | 0.129 | 0.99 | 0.312 | 0.87 | 0.686 | 0.87 | 0.518 | 0.9 | 0.292 | 0.9 | 0.061 | 1 | 0.226 | 1.06 | 0.111 | 0.81 | 0.062 |
| docosahexaenoylglycerol | | | | | | | | | | | | | | | | | | |
| (22:6) | | | | | | | | | | | | | | | | | | |
| 1- | 0.88 | 0.130 | 1.02 | 0.566 | 0.92 | 0.230 | 0.6 | 0.020 | 0.91 | 0.192 | 0.88 | 0.238 | 1.03 | 0.662 | 1.08 | 0.876 | 0.86 | 0.160 |
| palmitoleoylglycerophosphocholine | | | | | | | | | | | | | | | | | | |
| (16:1) | | | | | | | | | | | | | | | | | | |
| 2- | 0.84 | 0.131 | 0.89 | 0.112 | 0.8 | 0.066 | 0.54 | 0.106 | 0.85 | 0.306 | 0.95 | 0.916 | 0.89 | 0.116 | 0.94 | 0.191 | 0.86 | 0.361 |
| myristoylglycerophosphocholine | | | | | | | | | | | | | | | | | | |
| pantothenate | 0.78 | 0.133 | 0.84 | 0.191 | 0.73 | 0.087 | 1.14 | 0.783 | 0.87 | 0.286 | 1.05 | 0.671 | 0.84 | 0.219 | 0.91 | 0.414 | 0.8 | 0.236 |
| etiocholanolone | 0.81 | 0.135 | 0.61 | 0.017 | 0.58 | 0.017 | 0.32 | 0.113 | 0.95 | 0.938 | 1.6 | 0.238 | 0.59 | 0.011 | 0.63 | 0.044 | 0.93 | 0.460 |
| glucuronide | | | | | | | | | | | | | | | | | | |
| phenylalanine | 1.04 | 0.137 | 1 | 0.915 | 1.03 | 0.428 | 1.08 | 0.235 | 1.06 | 0.302 | 1.06 | 0.194 | 1 | 0.789 | 0.99 | 0.544 | 1.05 | 0.100 |
| cys-gly, oxidized | 0.87 | 0.139 | 0.74 | 0.002 | 0.71 | 0.005 | 0.74 | 0.188 | 0.57 | 0.023 | 0.76 | 0.400 | 0.76 | 0.003 | 0.75 | 0.004 | 0.94 | 0.749 |
| 1- | 0.79 | 0.141 | 0.8 | 0.084 | 0.71 | 0.052 | 0.65 | 0.283 | 0.64 | 0.031 | 0.78 | 0.146 | 0.82 | 0.127 | 0.86 | 0.181 | 0.83 | 0.339 |
| docosabentaenoylglycerophosphocholine | | | | | | | | | | | | | | | | | | |
| (22:5n6) | | | | | | | | | | | | | | | | | | |
| N-acetylputrescine | 1.17 | 0.142 | 1.2 | 0.068 | 1.28 | 0.038 | 1.07 | 0.665 | 1.31 | 0.076 | 1.1 | 0.741 | 1.19 | 0.076 | 1.16 | 0.131 | 1.11 | 0.477 |
| dodecanedioate | 1.33 | 0.144 | 0.92 | 0.527 | 1.17 | 0.182 | 1.93 | 0.250 | 1.49 | 0.050 | 1.68 | 0.066 | 0.89 | 0.698 | 0.83 | 0.893 | 1.4 | 0.197 |
| tryptophan | 1.05 | 0.145 | 0.98 | 0.580 | 1.02 | 0.597 | 1 | 0.975 | 1.06 | 0.340 | 1.09 | 0.151 | 0.98 | 0.458 | 0.96 | 0.295 | 1.06 | 0.087 |
| cholate | 2.57 | 0.149 | 3.27 | 0.024 | 4.54 | 0.024 | 1.45 | 0.930 | 3.85 | 0.691 | 1.19 | 0.165 | 3.22 | 0.018 | 2.87 | 0.053 | 1.58 | 0.483 |
| 2- | 0.82 | 0.150 | 0.93 | 0.307 | 0.82 | 0.141 | 0.42 | 0.007 | 0.72 | 0.003 | 0.75 | 0.004 | 0.96 | 0.454 | 1 | 0.552 | 0.82 | 0.231 |
| arachidonoylglycerophosphoinositol | | | | | | | | | | | | | | | | | | |
| TL22:1n9 (erucic acid) | 0.81 | 0.152 | 0.86 | 0.332 | 0.77 | 0.138 | 0.36 | 0.005 | 0.61 | 0.018 | 0.68 | 0.032 | 0.9 | 0.519 | 0.92 | 0.632 | 0.83 | 0.229 |
| azelate (nonanedioate) | 1.12 | 0.157 | 1.03 | 0.954 | 1.11 | 0.486 | 1.32 | 0.168 | 1.3 | 0.125 | 1.28 | 0.068 | 1.01 | 0.752 | 0.99 | 0.642 | 1.12 | 0.111 |
| gamma- | 1.08 | 0.157 | 0.98 | 0.440 | 1.05 | 0.783 | 1.14 | 0.742 | 1.07 | 0.782 | 1.09 | 0.325 | 0.98 | 0.386 | 0.95 | 0.206 | 1.1 | 0.061 |
| glutamyltryptophan | | | | | | | | | | | | | | | | | | |
| saccharin | 0.58 | 0.159 | 0.43 | 0.110 | 0.34 | 0.070 | 1.89 | 0.660 | 0.87 | 0.971 | 2.11 | 0.417 | 0.41 | 0.087 | 0.49 | 0.235 | 0.7 | 0.328 |
| indolelactate | 1.09 | 0.159 | 1.12 | 0.065 | 1.16 | 0.047 | 1.13 | 0.586 | 1.17 | 0.212 | 1.04 | 0.625 | 1.12 | 0.080 | 1.11 | 0.137 | 1.05 | 0.408 |
| trigonelline (N′- | 0.52 | 0.160 | 0.57 | 0.080 | 0.41 | 0.057 | 0.75 | 0.378 | 0.66 | 0.124 | 1.16 | 0.447 | 0.57 | 0.106 | 0.71 | 0.193 | 0.58 | 0.336 |
| methylnicotinate) | | | | | | | | | | | | | | | | | | |
| 4-ureidobutyrate | 0.86 | 0.162 | 0.84 | 0.088 | 0.78 | 0.059 | 0.6 | 0.235 | 0.72 | 0.291 | 0.85 | 0.700 | 0.85 | 0.103 | 0.87 | 0.172 | 0.9 | 0.396 |
| 2- | 0.89 | 0.165 | 1.07 | 0.389 | 0.96 | 0.732 | 0.65 | 0.008 | 0.88 | 0.190 | 0.81 | 0.047 | 1.09 | 0.259 | 1.13 | 0.155 | 0.85 | 0.076 |
| oleoylglycerophosphocholine | | | | | | | | | | | | | | | | | | |
| sphingosine 1- | 0.95 | 0.166 | 0.91 | 0.099 | 0.89 | 0.054 | 1.01 | 0.932 | 0.98 | 0.750 | 1.08 | 0.512 | 0.91 | 0.087 | 0.92 | 0.223 | 0.97 | 0.394 |
| phosphate | | | | | | | | | | | | | | | | | | |
| desmosterol | 1.16 | 0.167 | 1.11 | 0.287 | 1.21 | 0.136 | 1.54 | 0.031 | 1.63 | 0.035 | 1.51 | 0.059 | 1.08 | 0.462 | 1.06 | 0.512 | 1.14 | 0.279 |
| orotidine | 2.13 | 0.172 | 1.54 | 0.402 | 2.51 | 0.192 | 1.36 | 0.436 | 4.36 | 0.036 | 2.98 | 0.052 | 1.46 | 0.504 | 1.29 | 0.616 | 1.94 | 0.280 |
| octadecanedioate | 1.04 | 0.174 | 0.98 | 0.537 | 1.01 | 0.215 | 1.16 | 0.360 | 1.52 | 0.030 | 1.6 | 0.035 | 0.95 | 0.774 | 0.96 | 0.949 | 1.05 | 0.202 |
| allantoin | 1.14 | 0.180 | 0.83 | 0.149 | 0.95 | 0.999 | 1.75 | 0.198 | 1.18 | 0.661 | 1.47 | 0.174 | 0.8 | 0.107 | 0.77 | 0.043 | 1.23 | 0.060 |
| TL20:3n9 (mead acid) | 1.17 | 0.182 | 1.13 | 0.200 | 1.23 | 0.112 | 0.79 | 0.111 | 1.02 | 0.448 | 0.9 | 0.044 | 1.14 | 0.146 | 1.08 | 0.387 | 1.13 | 0.315 |
| alpha-tocopherol | 0.92 | 0.182 | 0.93 | 0.923 | 0.89 | 0.342 | 0.79 | 0.253 | 0.78 | 0.084 | 0.82 | 0.062 | 0.95 | 0.862 | 0.95 | 0.694 | 0.93 | 0.183 |
| pyridoxate | 0.49 | 0.182 | 0.6 | 0.094 | 0.41 | 0.077 | 1.67 | 0.586 | 0.69 | 0.539 | 1.16 | 0.935 | 0.6 | 0.094 | 0.78 | 0.218 | 0.53 | 0.338 |
| 1- | 1.24 | 0.183 | 1.18 | 0.167 | 1.33 | 0.095 | 1.31 | 0.535 | 1.64 | 0.102 | 1.42 | 0.285 | 1.16 | 0.221 | 1.12 | 0.308 | 1.19 | 0.371 |
| palmitoylglycerophosphoglycerol | | | | | | | | | | | | | | | | | | |
| 1- | 0.87 | 0.183 | 1.14 | 0.227 | 0.99 | 0.942 | 0.42 | 0.071 | 0.82 | 0.552 | 0.69 | 0.204 | 1.18 | 0.159 | 1.22 | 0.089 | 0.81 | 0.073 |
| eicosatrienoylglycerophosphoethanolamine | | | | | | | | | | | | | | | | | | |
| flavin adenine | 1.26 | 0.187 | 1.37 | 0.026 | 1.49 | 0.027 | 1.92 | 0.072 | 1.5 | 0.117 | 1.1 | 0.874 | 1.36 | 0.029 | 1.31 | 0.041 | 1.14 | 0.743 |
| dinucleotide (FAD) | | | | | | | | | | | | | | | | | | |
| N-acetyl-3- | 1.3 | 0.188 | 1.65 | 0.174 | 1.74 | 0.109 | 1.91 | 0.113 | 2.32 | 0.058 | 1.44 | 0.130 | 1.61 | 0.268 | 1.6 | 0.302 | 1.08 | 0.365 |
| methylhistidine | | | | | | | | | | | | | | | | | | |
| palmitoyl-oleoyl- | 1.07 | 0.188 | 1.1 | 0.059 | 1.13 | 0.054 | 1.05 | 0.731 | 1.17 | 0.144 | 1.07 | 0.591 | 1.1 | 0.073 | 1.09 | 0.127 | 1.03 | 0.430 |
| glycerophosphocholine | | | | | | | | | | | | | | | | | | |
| cysteine | 0.93 | 0.205 | 0.87 | 0.038 | 0.86 | 0.037 | 1.14 | 0.677 | 0.85 | 0.067 | 0.97 | 0.487 | 0.88 | 0.049 | 0.88 | 0.080 | 0.97 | 0.562 |
| pregnanediol-3- | 1.16 | 0.207 | 0.67 | 0.109 | 0.82 | 0.070 | 0.58 | 0.254 | 0.97 | 0.554 | 1.5 | 0.776 | 0.65 | 0.105 | 0.61 | 0.208 | 1.34 | 0.481 |
| glucuronide | | | | | | | | | | | | | | | | | | |
| N-acetylalanine | 1.04 | 0.209 | 1.02 | 0.442 | 1.04 | 0.209 | 1.1 | 0.070 | 1.06 | 0.235 | 1.05 | 0.436 | 1.02 | 0.498 | 1.01 | 0.727 | 1.04 | 0.288 |
| 4-ethylphenylsulfate | 0.52 | 0.218 | 0.52 | 0.075 | 0.38 | 0.075 | 0.21 | 0.551 | 0.18 | 0.360 | 0.3 | 0.769 | 0.62 | 0.086 | 0.65 | 0.147 | 0.58 | 0.449 |
| 2- | 0.83 | 0.220 | 0.94 | 0.276 | 0.83 | 0.165 | 0.41 | 0.083 | 0.75 | 0.192 | 0.78 | 0.379 | 0.97 | 0.336 | 1.01 | 0.438 | 0.82 | 0.371 |
| linolenoylglycerophosphocholine | | | | | | | | | | | | | | | | | | |
| (18:3n3) | | | | | | | | | | | | | | | | | | |
| alpha-CEHC | 1.1 | 0.225 | 0.26 | 0.018 | 0.34 | 0.031 | 0.58 | 0.511 | 0.33 | 0.060 | 1.31 | 0.277 | 0.25 | 0.034 | 0.23 | 0.055 | 1.48 | 0.549 |
| phenylalanylphenylalanine | 0.95 | 0.234 | 0.92 | 0.327 | 0.91 | 0.168 | 0.84 | 0.311 | 0.8 | 0.059 | 0.86 | 0.130 | 0.93 | 0.423 | 0.93 | 0.516 | 0.98 | 0.410 |
| ornithine | 1.07 | 0.237 | 1.04 | 0.360 | 1.09 | 0.192 | 1.57 | 0.007 | 1.23 | 0.063 | 1.19 | 0.108 | 1.03 | 0.499 | 1.02 | 0.578 | 1.06 | 0.365 |
| 1- | 0.87 | 0.240 | 0.92 | 0.213 | 0.84 | 0.147 | 0.59 | 0.080 | 0.91 | 0.486 | 0.99 | 0.982 | 0.92 | 0.218 | 0.96 | 0.333 | 0.88 | 0.439 |
| myristoylglycerophosphocholine | | | | | | | | | | | | | | | | | | |
| (14:0) | | | | | | | | | | | | | | | | | | |
| glucuronate | 1.01 | 0.241 | 1.04 | 0.249 | 1.03 | 0.161 | 1.16 | 0.330 | 1.31 | 0.037 | 1.28 | 0.113 | 1.02 | 0.335 | 1.04 | 0.451 | 1 | 0.365 |
| threonylphenylalanine | 0.89 | 0.241 | 0.72 | 0.106 | 0.71 | 0.086 | 0.51 | 0.108 | 0.52 | 0.029 | 0.7 | 0.116 | 0.74 | 0.162 | 0.72 | 0.184 | 0.98 | 0.524 |
| succinylcarnitine | 1.19 | 0.242 | 0.93 | 0.455 | 1.08 | 0.220 | 1.42 | 0.036 | 1.11 | 0.517 | 1.22 | 0.848 | 0.91 | 0.479 | 0.86 | 0.732 | 1.25 | 0.339 |
| 3-hydroxybutyrate | 1 | 0.243 | 1.49 | 0.109 | 1.33 | 0.099 | 1.59 | 0.281 | 2.03 | 0.150 | 1.39 | 0.570 | 1.46 | 0.128 | 1.59 | 0.182 | 0.84 | 0.492 |
| (BHBA) | | | | | | | | | | | | | | | | | | |
| homovanillate (HVA) | 1.09 | 0.245 | 1.15 | 0.036 | 1.18 | 0.046 | 1.22 | 0.269 | 1.23 | 0.159 | 1.08 | 0.676 | 1.14 | 0.045 | 1.13 | 0.078 | 1.04 | 0.586 |
| N-palmitoyl glycine | 0.93 | 0.246 | 1.04 | 0.791 | 0.98 | 0.637 | 0.86 | 0.148 | 0.97 | 0.249 | 0.92 | 0.061 | 1.05 | 0.686 | 1.08 | 0.521 | 0.91 | 0.172 |
| 5alpha-androstan- | 0.93 | 0.246 | 0.73 | 0.118 | 0.74 | 0.091 | 0.61 | 0.142 | 1.14 | 0.646 | 1.61 | 0.604 | 0.71 | 0.107 | 0.73 | 0.200 | 1.02 | 0.536 |
| 3beta,17alpha-diol | | | | | | | | | | | | | | | | | | |
| disulfate | | | | | | | | | | | | | | | | | | |
| 3-methoxytyrosine | 0.97 | 0.259 | 0.93 | 0.105 | 0.92 | 0.085 | 1 | 0.955 | 0.95 | 0.418 | 1.02 | 0.895 | 0.93 | 0.106 | 0.93 | 0.178 | 0.99 | 0.585 |
| erythronate | 1.03 | 0.270 | 1.08 | 0.117 | 1.08 | 0.114 | 1.02 | 0.861 | 1.03 | 0.866 | 0.95 | 0.395 | 1.08 | 0.098 | 1.08 | 0.197 | 1 | 0.501 |
| catechol sulfate | 0.95 | 0.273 | 1.13 | 0.300 | 1.06 | 0.205 | 1.02 | 0.675 | 1.08 | 0.092 | 0.95 | 0.218 | 1.14 | 0.366 | 1.18 | 0.465 | 0.9 | 0.407 |
| tyrosylglutamate | 1.27 | 0.273 | 1 | 0.941 | 1.2 | 0.471 | 2.64 | 0.023 | 1.75 | 0.144 | 1.82 | 0.106 | 0.96 | 0.862 | 0.92 | 0.769 | 1.31 | 0.265 |
| palmitoyl-oleoyl- | 0.99 | 0.286 | 1.07 | 0.096 | 1.05 | 0.104 | 1.39 | 0.496 | 1.23 | 0.338 | 1.15 | 0.734 | 1.06 | 0.114 | 1.09 | 0.197 | 0.96 | 0.508 |
| glycerophosphoglycerol | | | | | | | | | | | | | | | | | | |
| 3-hydroxylaurate | 1.07 | 0.286 | 1.15 | 0.087 | 1.16 | 0.094 | 1.19 | 0.213 | 1.32 | 0.081 | 1.16 | 0.379 | 1.14 | 0.113 | 1.14 | 0.147 | 1.02 | 0.579 |
| vanillic alcohol sulfate | 1.95 | 0.293 | 3.39 | 0.928 | 3.63 | 0.505 | 2.3 | 0.264 | 17.7 | 0.060 | 5.57 | 0.021 | 3.17 | 0.912 | 3.28 | 0.809 | 1.11 | 0.277 |
| 2-hydroxyhippurate | 1.76 | 0.297 | 0.44 | 0.238 | 0.8 | 0.935 | 10.87 | 0.680 | 7.05 | 0.229 | 17.3 | 0.021 | 0.41 | 0.161 | 0.36 | 0.112 | 2.24 | 0.136 |
| (salicylurate) | | | | | | | | | | | | | | | | | | |
| thyroxine | 0.93 | 0.319 | 1.28 | 0.170 | 1.13 | 0.778 | 1.01 | 0.527 | 1.17 | 0.635 | 0.91 | 0.781 | 1.29 | 0.162 | 1.36 | 0.075 | 0.83 | 0.116 |
| allo-isoleucine | 1.07 | 0.322 | 1.16 | 0.008 | 1.17 | 0.021 | 0.95 | 0.852 | 1.29 | 0.022 | 1.13 | 0.277 | 1.15 | 0.013 | 1.15 | 0.014 | 1.02 | 0.941 |
| 4-androsten- | 1 | 0.325 | 0.88 | 0.418 | 0.91 | 0.263 | 0.42 | 0.090 | 0.93 | 0.710 | 1.07 | 0.901 | 0.87 | 0.416 | 0.86 | 0.591 | 1.05 | 0.484 |
| 3alpha,17alpha-diol | | | | | | | | | | | | | | | | | | |
| monosulfate | | | | | | | | | | | | | | | | | | |
| S-methylcysteine | 1.09 | 0.332 | 1.1 | 0.174 | 1.15 | 0.146 | 1.27 | 0.245 | 1.34 | 0.017 | 1.23 | 0.072 | 1.09 | 0.239 | 1.08 | 0.261 | 1.06 | 0.625 |
| leucylglycine | 0.89 | 0.334 | 0.83 | 0.248 | 0.8 | 0.192 | 0.35 | 0.035 | 0.47 | 0.060 | 0.53 | 0.114 | 0.89 | 0.376 | 0.85 | 0.371 | 0.93 | 0.549 |
| glycohyocholate | 1.74 | 0.338 | 1.31 | 0.159 | 1.89 | 0.144 | 2.51 | 0.057 | 4.14 | 0.022 | 3.36 | 0.082 | 1.23 | 0.227 | 1.13 | 0.231 | 1.67 | 0.647 |
| hypotaurine | 0.9 | 0.343 | 0.74 | 0.020 | 0.73 | 0.044 | 0.86 | 0.559 | 0.72 | 0.063 | 0.97 | 0.527 | 0.74 | 0.026 | 0.74 | 0.032 | 0.98 | 0.865 |
| glycodeoxycholate | 1.28 | 0.344 | 1.17 | 0.431 | 1.36 | 0.994 | 2.85 | 0.049 | 2.27 | 0.274 | 2.01 | 0.087 | 1.13 | 0.299 | 1.1 | 0.256 | 1.24 | 0.165 |
| pregnen-diol disulfate | 1.21 | 0.345 | 1.25 | 0.285 | 1.36 | 0.211 | 1.25 | 0.353 | 1.9 | 0.038 | 1.57 | 0.073 | 1.21 | 0.424 | 1.2 | 0.419 | 1.13 | 0.551 |
| quinolinate | 1.09 | 0.345 | 1.32 | 0.159 | 1.31 | 0.153 | 2.03 | 0.252 | 1.71 | 0.084 | 1.32 | 0.250 | 1.3 | 0.210 | 1.33 | 0.223 | 0.99 | 0.651 |
| betaine | 0.97 | 0.345 | 0.92 | 0.062 | 0.92 | 0.073 | 1.05 | 0.525 | 0.94 | 0.532 | 1.02 | 0.763 | 0.91 | 0.059 | 0.92 | 0.104 | 1 | 0.775 |
| HWESASXX | 1.06 | 0.346 | 1.14 | 0.710 | 1.15 | 0.755 | 0.95 | 0.357 | 1.1 | 0.294 | 0.97 | 0.086 | 1.14 | 0.616 | 1.13 | 0.511 | 1.02 | 0.277 |
| fructose | 0.89 | 0.346 | 1.12 | 0.046 | 1 | 0.609 | 1.12 | 0.817 | 1.19 | 0.169 | 1.07 | 0.887 | 1.11 | 0.049 | 1.19 | 0.010 | 0.84 | 0.098 |
| octanoylcarnitine | 1 | 0.347 | 0.8 | 0.047 | 0.84 | 0.062 | 0.78 | 0.336 | 0.78 | 0.144 | 0.97 | 0.541 | 0.8 | 0.063 | 0.78 | 0.090 | 1.08 | 0.761 |
| cystine | 1.04 | 0.357 | 0.98 | 0.768 | 1.01 | 0.661 | 1.19 | 0.017 | 1.01 | 0.938 | 1.04 | 0.882 | 0.98 | 0.761 | 0.96 | 0.481 | 1.05 | 0.282 |
| glucoheptose | 0.49 | 0.358 | 1.11 | 0.193 | 0.67 | 0.187 | 2.52 | 0.137 | 2.5 | 0.053 | 2.38 | 0.196 | 1.05 | 0.238 | 1.65 | 0.266 | 0.4 | 0.611 |
| alpha-CEHC sulfate | 1.03 | 0.361 | 0.44 | 0.049 | 0.53 | 0.080 | 1.12 | 0.746 | 0.38 | 0.075 | 0.83 | 0.279 | 0.45 | 0.078 | 0.41 | 0.086 | 1.28 | 0.740 |
| theobromine | 0.85 | 0.366 | 0.91 | 0.266 | 0.83 | 0.227 | 0.69 | 0.381 | 0.72 | 0.010 | 0.76 | 0.021 | 0.94 | 0.363 | 0.96 | 0.362 | 0.86 | 0.586 |
| stigmasterol | 0.88 | 0.367 | 1.02 | 0.653 | 0.92 | 0.743 | 0.84 | 0.259 | 0.67 | 0.132 | 0.63 | 0.057 | 1.07 | 0.459 | 1.07 | 0.411 | 0.86 | 0.277 |
| taurodeoxycholate | 1.37 | 0.374 | 1.54 | 0.977 | 1.74 | 0.629 | 2.46 | 0.073 | 2.08 | 0.284 | 1.38 | 0.212 | 1.51 | 0.815 | 1.46 | 0.748 | 1.19 | 0.324 |
| 3beta,7alpha- | 1.01 | 0.375 | 0.94 | 0.941 | 0.96 | 0.612 | 2.36 | 0.037 | 1.67 | 0.143 | 1.84 | 0.091 | 0.9 | 0.730 | 0.93 | 0.676 | 1.03 | 0.327 |
| dihydroxy-5- | | | | | | | | | | | | | | | | | | |
| cholestenoate | | | | | | | | | | | | | | | | | | |
| phenylalanyltryptophan | 0.92 | 0.382 | 1.04 | 0.914 | 0.97 | 0.564 | 0.72 | 0.183 | 0.82 | 0.106 | 0.77 | 0.062 | 1.06 | 0.921 | 1.08 | 0.859 | 0.9 | 0.367 |
| acetylcarnitine | 1.09 | 0.384 | 1.11 | 0.151 | 1.15 | 0.149 | 1.42 | 0.025 | 1.22 | 0.197 | 1.11 | 0.527 | 1.1 | 0.182 | 1.09 | 0.215 | 1.06 | 0.726 |
| N2,N5- | 1.21 | 0.385 | 1.19 | 0.442 | 1.31 | 0.314 | 1.88 | 0.057 | 1.63 | 0.335 | 1.41 | 0.562 | 1.16 | 0.488 | 1.13 | 0.602 | 1.16 | 0.529 |
| diacetylornithine | | | | | | | | | | | | | | | | | | |
| 1- | 0.96 | 0.390 | 1.02 | 0.930 | 0.98 | 0.585 | 0.68 | 0.008 | 0.86 | 0.133 | 0.84 | 0.089 | 1.03 | 0.888 | 1.03 | 0.857 | 0.95 | 0.373 |
| arachidonoylglycerophosphoethanolamine | | | | | | | | | | | | | | | | | | |
| serylalanine | 0.9 | 0.392 | 0.9 | 0.250 | 0.85 | 0.227 | 0.58 | 0.144 | 0.74 | 0.085 | 0.81 | 0.212 | 0.91 | 0.316 | 0.92 | 0.337 | 0.93 | 0.630 |
| 2- | 0.92 | 0.410 | 1.01 | 0.986 | 0.94 | 0.592 | 0.68 | 0.013 | 0.92 | 0.171 | 0.91 | 0.108 | 1.02 | 0.876 | 1.04 | 0.748 | 0.9 | 0.376 |
| arachidonoylglycerophosphocholine | | | | | | | | | | | | | | | | | | |
| cis-4-decenoyl carnitine | 0.96 | 0.413 | 0.87 | 0.070 | 0.87 | 0.102 | 0.88 | 0.554 | 0.84 | 0.134 | 0.97 | 0.567 | 0.87 | 0.087 | 0.87 | 0.109 | 1 | 0.817 |
| 2-hydroxybutyrate | 1.1 | 0.424 | 1.25 | 0.054 | 1.27 | 0.084 | 1.1 | 0.590 | 1.36 | 0.109 | 1.1 | 0.561 | 1.24 | 0.065 | 1.24 | 0.066 | 1.02 | 0.981 |
| (AHB) | | | | | | | | | | | | | | | | | | |
| N-acetylhistidine | 1.14 | 0.424 | 1.18 | 0.129 | 1.25 | 0.152 | 1.86 | 0.094 | 1.42 | 0.215 | 1.22 | 0.544 | 1.16 | 0.160 | 1.15 | 0.193 | 1.09 | 0.747 |
| decanoylcarnitine | 1 | 0.440 | 0.81 | 0.045 | 0.85 | 0.078 | 0.82 | 0.410 | 0.83 | 0.176 | 1.03 | 0.727 | 0.81 | 0.054 | 0.79 | 0.071 | 1.08 | 0.917 |
| 4-hydroxyhippurate | 0.87 | 0.445 | 0.78 | 0.107 | 0.74 | 0.148 | 1.31 | 0.707 | 1.16 | 0.997 | 1.54 | 0.291 | 0.75 | 0.085 | 0.79 | 0.153 | 0.94 | 0.792 |
| TL15:0 (pentadecanoic | 0.98 | 0.446 | 0.91 | 0.012 | 0.92 | 0.036 | 0.95 | 0.558 | 0.92 | 0.194 | 1.01 | 0.974 | 0.91 | 0.013 | 0.91 | 0.021 | 1.01 | 0.932 |
| acid) | | | | | | | | | | | | | | | | | | |
| chenodeoxycholate | 1.9 | 0.446 | 2.45 | 0.078 | 3.01 | 0.091 | 1.29 | 0.333 | 2.54 | 0.455 | 1.04 | 0.551 | 2.44 | 0.072 | 2.24 | 0.099 | 1.34 | 0.928 |
| 12-HETE | 0.83 | 0.451 | 0.69 | 0.071 | 0.66 | 0.120 | 0.94 | 0.568 | 0.58 | 0.521 | 0.83 | 0.837 | 0.7 | 0.068 | 0.71 | 0.091 | 0.92 | 0.892 |
| leucylalanine | 0.81 | 0.456 | 0.7 | 0.085 | 0.65 | 0.135 | 0.45 | 0.228 | 0.55 | 0.236 | 0.76 | 0.688 | 0.72 | 0.098 | 0.73 | 0.105 | 0.89 | 0.882 |
| TL18:3n6 (g-linolenic | 0.95 | 0.462 | 0.92 | 0.189 | 0.9 | 0.215 | 0.69 | 0.030 | 0.79 | 0.040 | 0.85 | 0.100 | 0.93 | 0.291 | 0.93 | 0.263 | 0.97 | 0.730 |
| acid) | | | | | | | | | | | | | | | | | | |
| serylleucine | 0.89 | 0.464 | 0.81 | 0.196 | 0.77 | 0.218 | 0.32 | 0.054 | 0.52 | 0.033 | 0.61 | 0.082 | 0.85 | 0.299 | 0.82 | 0.255 | 0.94 | 0.761 |
| 4-hydroxybenzoate | 1.06 | 0.465 | 0.49 | 0.179 | 0.58 | 0.190 | 1.85 | 0.080 | 2.03 | 0.841 | 4.45 | 0.201 | 0.46 | 0.146 | 0.45 | 0.256 | 1.29 | 0.774 |
| X-12093 | 1.24 | 0.469 | 1.17 | 0.104 | 1.32 | 0.135 | 1.62 | 0.082 | 1.25 | 0.408 | 1.07 | 0.971 | 1.17 | 0.109 | 1.11 | 0.148 | 1.19 | 0.873 |
| ADSGEGDFXAEGGG | 1.11 | 0.471 | 1.21 | 0.043 | 1.24 | 0.063 | 1.17 | 0.423 | 1.28 | 0.161 | 1.07 | 0.779 | 1.2 | 0.051 | 1.19 | 0.069 | 1.04 | 0.970 |
| VR | | | | | | | | | | | | | | | | | | |
| N2-acetyllysine | 1.16 | 0.485 | 1.18 | 0.085 | 1.27 | 0.111 | 1.62 | 0.131 | 1.44 | 0.090 | 1.24 | 0.550 | 1.17 | 0.104 | 1.15 | 0.129 | 1.1 | 0.929 |
| palmitoyl-linoleoyl- | 1.03 | 0.494 | 1.02 | 0.372 | 1.04 | 0.336 | 1.13 | 0.485 | 1.2 | 0.023 | 1.18 | 0.041 | 1.01 | 0.546 | 1.02 | 0.509 | 1.02 | 0.654 |
| glycerophosphocholine | | | | | | | | | | | | | | | | | | |
| 4-methylcatechol | 1.2 | 0.499 | 0.81 | 0.136 | 0.98 | 0.572 | 1.68 | 0.986 | 1.45 | 0.818 | 1.85 | 0.146 | 0.78 | 0.103 | 0.75 | 0.072 | 1.31 | 0.193 |
| sulfate | | | | | | | | | | | | | | | | | | |
| docosatrienoate | 1.04 | 0.515 | 1.16 | 0.355 | 1.15 | 0.348 | 1.27 | 0.342 | 1.43 | 0.065 | 1.25 | 0.109 | 1.14 | 0.469 | 1.16 | 0.427 | 0.99 | 0.726 |
| (22:3n3) | | | | | | | | | | | | | | | | | | |
| 9,10-DiHOME | 0.81 | 0.517 | 1.07 | 0.700 | 0.9 | 0.535 | 1.5 | 0.213 | 1.84 | 0.070 | 1.79 | 0.063 | 1.03 | 0.919 | 1.18 | 0.867 | 0.76 | 0.565 |
| 12,13-DiHOME | 0.87 | 0.538 | 1.03 | 0.440 | 0.92 | 0.411 | 1.77 | 0.040 | 1.71 | 0.053 | 1.73 | 0.071 | 0.99 | 0.649 | 1.09 | 0.564 | 0.85 | 0.667 |
| valylarginine | 0.92 | 0.542 | 0.79 | 0.126 | 0.78 | 0.186 | 0.4 | 0.103 | 0.59 | 0.033 | 0.72 | 0.150 | 0.81 | 0.174 | 0.79 | 0.154 | 0.99 | 0.942 |
| valylleucine | 0.92 | 0.550 | 0.83 | 0.247 | 0.81 | 0.286 | 0.71 | 0.163 | 0.53 | 0.054 | 0.61 | 0.102 | 0.87 | 0.383 | 0.83 | 0.311 | 0.98 | 0.812 |
| 1-stearoylglycerol | 0.89 | 0.554 | 1.16 | 0.127 | 1.02 | 0.600 | 0.82 | 0.978 | 1.3 | 0.443 | 1.13 | 0.924 | 1.15 | 0.137 | 1.25 | 0.073 | 0.82 | 0.276 |
| (18:0) | | | | | | | | | | | | | | | | | | |
| 3-hydroxyhippurate | 0.92 | 0.555 | 0.88 | 0.072 | 0.85 | 0.147 | 1.05 | 0.612 | 0.86 | 0.035 | 0.97 | 0.271 | 0.88 | 0.091 | 0.9 | 0.072 | 0.95 | 0.922 |
| xanthine | 1.07 | 0.556 | 0.94 | 0.346 | 1.01 | 0.769 | 1.39 | 0.381 | 1.24 | 0.295 | 1.34 | 0.086 | 0.92 | 0.231 | 0.91 | 0.237 | 1.1 | 0.303 |
| N1-methyladenosine | 0.99 | 0.569 | 1.04 | 0.418 | 1.02 | 0.832 | 0.87 | 0.138 | 0.96 | 0.304 | 0.91 | 0.064 | 1.05 | 0.315 | 1.05 | 0.300 | 0.97 | 0.345 |
| docosapentaenoate (n3 | 0.97 | 0.590 | 1.06 | 0.594 | 1.02 | 0.990 | 1 | 0.805 | 1.07 | 0.882 | 1.01 | 0.526 | 1.06 | 0.556 | 1.08 | 0.453 | 0.94 | 0.441 |
| DPA; 22:5n3) | | | | | | | | | | | | | | | | | | |
| 10-undecenoate | 1.05 | 0.591 | 0.99 | 0.687 | 1.03 | 0.974 | 1.26 | 0.725 | 1.22 | 0.261 | 1.25 | 0.083 | 0.98 | 0.567 | 0.97 | 0.556 | 1.06 | 0.460 |
| (11:1n1) | | | | | | | | | | | | | | | | | | |
| stearoyl sphingomyelin | 1 | 0.593 | 1.04 | 0.691 | 1.03 | 0.971 | 0.83 | 0.199 | 0.91 | 0.187 | 0.86 | 0.069 | 1.05 | 0.538 | 1.04 | 0.566 | 0.99 | 0.473 |
| (d18:1/18:0) | | | | | | | | | | | | | | | | | | |
| 2-keto-3-deoxy- | 0.95 | 0.594 | 0.76 | 0.309 | 0.77 | 0.839 | 1.5 | 0.176 | 1.24 | 0.229 | 1.69 | 0.054 | 0.73 | 0.206 | 0.75 | 0.195 | 1.03 | 0.392 |
| gluconate | | | | | | | | | | | | | | | | | | |
| 2-ethylphenylsulfate | 0.97 | 0.597 | 2.12 | 0.149 | 1.61 | 0.543 | 5.42 | 0.455 | 4.43 | 0.258 | 2.19 | 0.814 | 2.03 | 0.159 | 2.51 | 0.078 | 0.64 | 0.233 |
| glycolate | 1.03 | 0.606 | 1 | 0.908 | 1.02 | 0.698 | 1.15 | 0.099 | 1.09 | 0.180 | 1.1 | 0.141 | 0.99 | 0.934 | 0.99 | 0.968 | 1.03 | 0.630 |
| (hydroxyacetate) | | | | | | | | | | | | | | | | | | |
| 3-indoxyl sulfate | 1.05 | 0.611 | 0.9 | 0.123 | 0.96 | 0.498 | 1.36 | 0.768 | 0.92 | 0.266 | 1.02 | 0.835 | 0.9 | 0.133 | 0.87 | 0.066 | 1.1 | 0.241 |
| 1- | 0.96 | 0.621 | 1.08 | 0.134 | 1.03 | 0.570 | 0.89 | 0.497 | 0.96 | 0.594 | 0.88 | 0.122 | 1.09 | 0.094 | 1.11 | 0.079 | 0.93 | 0.318 |
| docosahexaenoylglycerophosphoethanolamine | | | | | | | | | | | | | | | | | | |
| arabonate | 1.05 | 0.627 | 1.09 | 0.254 | 1.11 | 0.310 | 1.35 | 0.127 | 1.32 | 0.053 | 1.23 | 0.117 | 1.08 | 0.352 | 1.09 | 0.290 | 1.02 | 0.946 |
| pregnanolone/allopregnanolone | 1.43 | 0.632 | 1.11 | 0.618 | 1.43 | 0.972 | 0.47 | 0.065 | 1.7 | 0.831 | 1.58 | 0.546 | 1.08 | 0.572 | 1 | 0.499 | 1.43 | 0.493 |
| sulfate | | | | | | | | | | | | | | | | | | |
| serotonin (5HT) | 0.78 | 0.639 | 0.82 | 0.761 | 0.72 | 0.631 | 0.28 | 0.029 | 0.46 | 0.066 | 0.52 | 0.057 | 0.88 | 0.990 | 0.89 | 0.889 | 0.81 | 0.691 |
| 7-methylguanine | 1.02 | 0.644 | 1.07 | 0.046 | 1.07 | 0.140 | 0.99 | 0.994 | 1.03 | 0.656 | 0.96 | 0.619 | 1.08 | 0.040 | 1.07 | 0.056 | 0.99 | 0.884 |
| 1,6-anhydroglucose | 0.89 | 0.657 | 0.69 | 0.092 | 0.69 | 0.204 | 1.37 | 0.897 | 1.12 | 0.489 | 1.67 | 0.431 | 0.67 | 0.086 | 0.7 | 0.107 | 1 | 0.943 |
| homostachydrine | 1.07 | 0.664 | 0.76 | 0.050 | 0.85 | 0.360 | 0.68 | 0.327 | 0.78 | 0.394 | 1.03 | 0.924 | 0.76 | 0.052 | 0.72 | 0.028 | 1.17 | 0.267 |
| methyl indole-3-acetate | 0.98 | 0.680 | 1.29 | 0.377 | 1.18 | 0.447 | 1.59 | 0.031 | 1.72 | 0.103 | 1.36 | 0.178 | 1.26 | 0.471 | 1.34 | 0.409 | 0.88 | 0.895 |
| phenylalanylleucine | 1.19 | 0.688 | 0.79 | 0.039 | 0.96 | 0.250 | 1.27 | 0.510 | 1.07 | 0.539 | 1.38 | 0.361 | 0.77 | 0.032 | 0.73 | 0.020 | 1.31 | 0.187 |
| 4- | 1.09 | 0.703 | 1.23 | 0.068 | 1.24 | 0.170 | 0.88 | 0.261 | 0.98 | 0.344 | 0.78 | 0.017 | 1.25 | 0.039 | 1.22 | 0.076 | 1.01 | 0.811 |
| hydroxyphenylpyruvate | | | | | | | | | | | | | | | | | | |
| 3beta,7beta-dihydroxy- | 1.03 | 0.706 | 0.91 | 0.279 | 0.95 | 0.324 | 1.13 | 0.758 | 1.14 | 0.439 | 1.27 | 0.019 | 0.9 | 0.222 | 0.89 | 0.323 | 1.07 | 0.958 |
| 5-cholestenoate | | | | | | | | | | | | | | | | | | |
| erythritol | 0.24 | 0.709 | 0.83 | 0.131 | 0.37 | 0.643 | 0.15 | 0.667 | 0.28 | 0.710 | 0.28 | 0.392 | 1 | 0.072 | 2.11 | 0.042 | 0.18 | 0.407 |
| phenylalanylglutamate | 1.28 | 0.726 | 0.83 | 0.135 | 1.05 | 0.440 | 2.31 | 0.414 | 0.8 | 0.997 | 0.96 | 0.361 | 0.83 | 0.108 | 0.75 | 0.089 | 1.4 | 0.328 |
| ethyl glucuronide | 0.67 | 0.736 | 0.6 | 0.342 | 0.5 | 0.472 | 1 | | 1.24 | 0.160 | 2.18 | 0.004 | 0.57 | 0.280 | 0.66 | 0.411 | 0.76 | 0.903 |
| L-urobilin | 1.88 | 0.777 | 2.12 | 0.258 | 2.75 | 0.376 | 2.88 | 0.248 | 5.78 | 0.073 | 2.88 | 0.181 | 2 | 0.330 | 1.9 | 0.263 | 1.45 | 0.885 |
| 5alpha-pregnan-3(alpha | 1.17 | 0.784 | 1.18 | 0.715 | 1.27 | 0.947 | 1.83 | 0.228 | 4.02 | 0.080 | 3.62 | 0.073 | 1.11 | 0.910 | 1.14 | 0.636 | 1.12 | 0.674 |
| or beta),20beta-diol | | | | | | | | | | | | | | | | | | |
| disulfate | | | | | | | | | | | | | | | | | | |
| TL18:1n7 (avaccenic | 1 | 0.832 | 1.01 | 0.721 | 1.01 | 0.917 | 0.99 | 0.804 | 0.93 | 0.128 | 0.91 | 0.050 | 1.02 | 0.532 | 1.02 | 0.654 | 0.99 | 0.716 |
| acid) | | | | | | | | | | | | | | | | | | |
| p-cresol-glucuronide | 1.3 | 0.834 | 0.8 | 0.130 | 1.03 | 0.401 | 2.02 | 0.861 | 1.58 | 0.836 | 2.06 | 0.436 | 0.77 | 0.110 | 0.72 | 0.091 | 1.44 | 0.411 |
| 3-carboxy-4-methyl-5- | 1.61 | 0.836 | 1.2 | 0.063 | 1.67 | 0.254 | 3.98 | 0.578 | 1.66 | 0.546 | 1.42 | 0.610 | 1.17 | 0.057 | 1.05 | 0.049 | 1.59 | 0.356 |
| propyl-2- | | | | | | | | | | | | | | | | | | |
| furanpropanoate | | | | | | | | | | | | | | | | | | |
| (CMPF) | | | | | | | | | | | | | | | | | | |
| 13-HODE + 9-HODE | 0.87 | 0.841 | 0.92 | 0.448 | 0.84 | 0.754 | 1.3 | 0.726 | 1.39 | 0.340 | 1.55 | 0.051 | 0.9 | 0.360 | 0.97 | 0.362 | 0.88 | 0.632 |
| indoleacetate | 0.92 | 0.861 | 1.22 | 0.217 | 1.08 | 0.543 | 1.83 | 0.007 | 1.79 | 0.015 | 1.52 | 0.038 | 1.18 | 0.337 | 1.3 | 0.155 | 0.84 | 0.537 |
| 2- | 0.93 | 0.866 | 1.09 | 0.878 | 1.02 | 0.994 | 0.58 | 0.029 | 1.03 | 0.384 | 0.94 | 0.227 | 1.1 | 0.786 | 1.14 | 0.822 | 0.89 | 0.812 |
| palmitoleoylglycerophosphocholine | | | | | | | | | | | | | | | | | | |
| cyclo(pro-pro) | 1.04 | 0.897 | 1.11 | 0.064 | 1.12 | 0.257 | 1.37 | 0.185 | 1.06 | 0.697 | 0.95 | 0.565 | 1.12 | 0.056 | 1.11 | 0.055 | 1.01 | 0.438 |
| gamma-glutamylalanine | 1.03 | 0.907 | 1.02 | 0.844 | 1.04 | 0.842 | 0.82 | 0.303 | 0.97 | 0.671 | 0.94 | 0.521 | 1.03 | 0.784 | 1.02 | 0.864 | 1.02 | 0.967 |
| delta-tocopherol | 1.05 | 0.913 | 1.22 | 0.011 | 1.2 | 0.106 | 1.26 | 0.732 | 1.22 | 0.535 | 1 | 0.264 | 1.22 | 0.009 | 1.23 | 0.010 | 0.97 | 0.483 |
| palmitoyl | 1 | 0.914 | 1.04 | 0.078 | 1.03 | 0.255 | 1.01 | 0.906 | 1.02 | 0.748 | 0.98 | 0.407 | 1.04 | 0.068 | 1.04 | 0.076 | 0.99 | 0.652 |
| sphingomyelin | | | | | | | | | | | | | | | | | | |
| (d18:1/16:0) | | | | | | | | | | | | | | | | | | |
| stearoyl-arachidonoyl- | 1 | 0.917 | 0.92 | 0.102 | 0.94 | 0.276 | 0.79 | 0.166 | 0.83 | 0.150 | 0.89 | 0.395 | 0.93 | 0.139 | 0.91 | 0.090 | 1.03 | 0.619 |
| glycerophosphocholine | | | | | | | | | | | | | | | | | | |
| 2- | 0.96 | 0.917 | 1.01 | 0.762 | 0.98 | 0.909 | 0.49 | 0.009 | 0.79 | 0.047 | 0.77 | 0.029 | 1.04 | 0.939 | 1.03 | 0.710 | 0.95 | 0.824 |
| arachidonoylglycerophosphoethanolamine | | | | | | | | | | | | | | | | | | |
| 4-vinylguaiacol sulfate | 0.97 | 0.932 | 0.78 | 0.997 | 0.8 | 0.955 | 3.33 | 0.069 | 2.65 | 0.241 | 3.62 | 0.171 | 0.73 | 0.869 | 0.76 | 0.978 | 1.05 | 0.929 |
| malonate | 0.9 | 0.971 | 0.99 | 0.100 | 0.92 | 0.313 | 1.43 | 0.058 | 1.4 | 0.091 | 1.45 | 0.366 | 0.96 | 0.139 | 1.03 | 0.103 | 0.89 | 0.659 |
| (propanedioate) | | | | | | | | | | | | | | | | | | |
| glycolithocholate | 1.03 | 0.978 | 0.98 | 0.299 | 1.01 | 0.486 | 2.38 | 0.093 | 1.79 | 0.621 | 1.89 | 0.128 | 0.95 | 0.240 | 0.97 | 0.271 | 1.04 | 0.685 |
| sulfate | | | | | | | | | | | | | | | | | | |
| 7-alpha-hydroxy-3-oxo- | 0.99 | 0.983 | 1.17 | 0.112 | 1.12 | 0.323 | 1.18 | 0.311 | 1.29 | 0.139 | 1.11 | 0.391 | 1.16 | 0.147 | 1.2 | 0.086 | 0.93 | 0.560 |
| 4-cholestenoate (7- | | | | | | | | | | | | | | | | | | |
| Hoca) | | | | | | | | | | | | | | | | | | |
| seryltyrosine | 1.11 | 0.987 | 0.73 | 0.043 | 0.85 | 0.181 | 1.18 | 0.514 | 0.9 | 0.274 | 1.25 | 0.832 | 0.72 | 0.043 | 0.68 | 0.034 | 1.24 | 0.442 |
| N-acetyl-aspartyl- | 0.95 | 0.992 | 0.86 | 0.042 | 0.86 | 0.311 | 0.92 | 0.711 | 0.76 | 0.070 | 0.87 | 0.284 | 0.87 | 0.068 | 0.86 | 0.024 | 0.99 | 0.597 |
| glutamate (NAAG) | | | | | | | | | | | | | | | | | | |
| succinate | 0.93 | 0.994 | 0.98 | 0.623 | 0.93 | 0.816 | 1.24 | 0.014 | 1.16 | 0.045 | 1.19 | 0.048 | 0.97 | 0.884 | 1.01 | 0.585 | 0.92 | 0.912 |

Distinguishing Fibrosis Stages 0-1 from Fibrosis Stages 2-4

To assess the performance of several commonly measured clinical parameters (Age, Type 2 Diabetes, BMI, HDL Cholesterol, Gender, Fructose, and Past Alcohol Use) for distinguishing fibrosis stages 0-1 samples from stages 2-4 samples, logistic regression and area under the curve (AUC) analyses were performed. The AUCs calculated for the individual clinical parameters ranged from 0.5079 for BMI to 0.6096 for Type 2 Diabetes. The data are shown in Table 13. A total of 127 combinations of these seven clinical parameters are possible, and all 127 possible combinatorial models using these clinical parameters were evaluated. The highest AUC obtained was 0.6663, and it was derived from a model that fit all seven clinical parameters.

TABLE 13
AUC values for select clinical parameters for distinguishing
fibrosis stage 0-1 from fibrosis stage 2-4 patient samples

| Clinical Parameter | AUC |
|---|---|
| Age | 0.5598 |
| Type 2 Diabetes | 0.6096 |
| BMI | 0.5079 |
| HDL Cholesterol | 0.5984 |
| Gender | 0.5454 |
| Fructose | 0.5464 |
| Past Alcohol Use | 0.5221 |

Logistic regression models and area under the curve (AUC) were also used to assess the performance of individual metabolites for distinguishing the fibrosis stage 0-1 samples from fibrosis stage 2-4 samples. Logistic regression analysis was performed on the measured values obtained for all 1151 metabolites detected in the samples. Metabolites with an AUC of >0.600 for distinguishing fibrosis stage 0-1 from fibrosis stage 2-4 patient samples were identified and are presented in Table 14. Of these, 114 metabolites have individual AUCs greater than the AUC of 0.6096 obtained for Type 2 Diabetes, the top clinical parameter. Further, eight metabolites, X-14662, ribose, I-urobilinogen, X-12850, malate, glutarate (pentanedioate), 2-aminoheptanoate, and X-15497, have an AUC greater than 0.6663, which is the AUC calculated from the best model using all 7 clinical parameters of Age, Type 2 Diabetes, BMI, HDL Cholesterol, Gender, Fructose, and Past Alcohol Use. The metabolites and data are listed in Table 14.

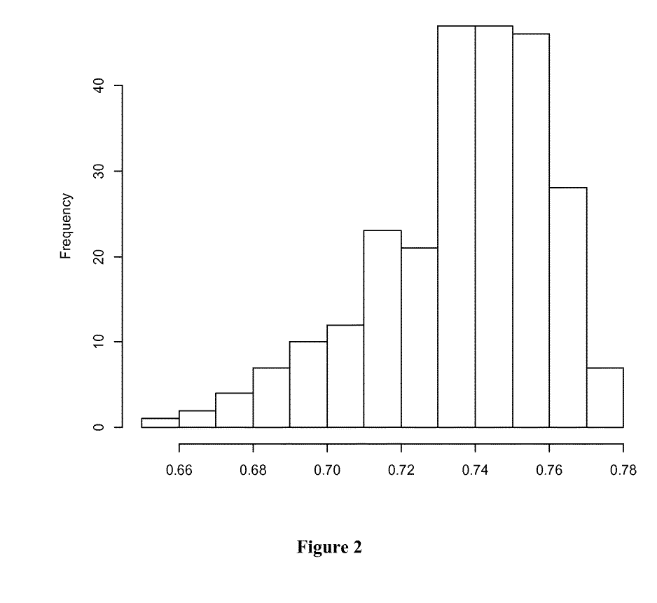

A total of 255 combinations using X-14662, ribose, I-urobilinogen, X-12850, malate, glutarate (pentanedioate), 2-aminoheptanoate, and X-15497 (the eight metabolites with an AUC >0.6663) are possible and all 255 possible combinatorial models for separating fibrosis stage 0-1 from fibrosis stage 2-4 were evaluated. The AUCs that were calculated for each model resulting from fitting all possible model combinations of the eight metabolites range from 0.6523 to 0.7774 and the data are shown in FIG. 2. The average AUC of all possible model combinations was 0.75, which is higher than the highest AUC obtained using any model consisting of only clinical parameters.

TABLE 14
AUC of individual metabolites for distinguishing fibrosis
stage 0-1 from fibrosis stage 2-4 patient samples

| Biochemical Name | AUC | Biochemical Name | AUC |
|---|---|---|---|
| glutarate (pentanedioate) | 0.671 | oleoyl-sphingomyelin | 0.621 |
| I-urobilinogen | 0.687 | X-11491 | 0.621 |
| fucose | 0.614 | 1- | 0.62 |
| | | arachidonoylglycerophosphoinositol | |
| 3-hydroxydecanoate | 0.613 | hexadecanedioate | 0.62 |
| 3-hydroxyoctanoate | 0.656 | N-acetylcitrulline | 0.619 |
| X-11871 | 0.663 | homoarginine | 0.619 |
| X-12850 | 0.683 | N-acetylmethionine | 0.618 |
| X-18889 | 0.632 | caproate (6:0) | 0.618 |
| 16a-hydroxy DHEA 3-sulfate | 0.63 | X-21431 | 0.617 |
| 2-aminoheptanoate | 0.671 | X-11905 | 0.617 |
| X-19561 | 0.663 | X-21736 | 0.617 |
| X-21471 | 0.638 | X-13429 | 0.617 |
| cyclo(L-phe-L-pro) | 0.611 | X-12127 | 0.617 |
| taurocholate | 0.622 | X-12193 | 0.616 |
| glycocholate | 0.637 | 1-linoleoylglycerol (1-monolinolein) | 0.614 |
| taurochenodeoxycholate | 0.617 | glucuronate | 0.614 |
| glycochenodeoxycholate | 0.648 | glucose | 0.614 |
| isoleucine | 0.665 | N6-acetyllysine | 0.613 |
| glutamate | 0.659 | TL24:0 (lignoceric acid) | 0.613 |
| alpha-ketoglutarate | 0.639 | cyclo(leu-pro) | 0.613 |
| X-14662 | 0.693 | eicosenoyl-sphingomyelin | 0.613 |
| ribose | 0.692 | tetradecanedioate | 0.612 |
| malate | 0.677 | 2-hydroxy-3-methylvalerate | 0.612 |
| X-15497 | 0.668 | gamma-CEHC | 0.612 |
| X-12263 | 0.664 | X-12824 | 0.612 |
| 1-stearoylglycerophosphoinositol | 0.663 | dimethylglycine | 0.612 |
| fumarate | 0.661 | phenylalanylserine | 0.612 |
| N-methylproline | 0.661 | 1- | 0.612 |
| | | eicosatrienoylglycerophosphocholine | |
| | | (20:3) | |
| gamma-glutamylisoleucine | 0.659 | X-17178 | 0.611 |
| X-18922 | 0.654 | X-21408 | 0.611 |
| threonate | 0.654 | cortisol | 0.611 |
| X-22102 | 0.653 | X-14314 | 0.611 |
| X-11537 | 0.653 | xylitol | 0.611 |
| X-21893 | 0.652 | cysteine-glutamione disulfide | 0.611 |
| X-12739 | 0.651 | S-adenosylhomocysteine (SAH) | 0.61 |
| X-17453 | 0.651 | 2-pyrrolidinone | 0.61 |
| X-17145 | 0.65 | 1-linoleoylglycerophosphocholine | 0.61 |
| | | (18:2n6) | |
| maleate (cis-Butenedioate) | 0.65 | X-23314 | 0.61 |
| alpha-glutamyltyrosine | 0.649 | X-11529 | 0.609 |
| X-13529 | 0.648 | 3,7-dimethylurate | 0.609 |
| 2-stearoylglycerophosphoinositol | 0.647 | glycoursodeoxycholate | 0.609 |
| oxalate (ethanedioate) | 0.645 | dihydroferulic acid | 0.609 |
| X-21892 | 0.644 | coprostanol | 0.609 |
| gamma-glutamylvaline | 0.643 | 7-methylxanmine | 0.608 |
| X-14302 | 0.642 | inosine | 0.608 |
| X-14658 | 0.64 | X-12122 | 0.608 |
| pelargonate (9:0) | 0.639 | ursodeoxycholate | 0.607 |
| 3-methylglutarylcarnitine | 0.639 | X-13728 | 0.607 |
| X-11564 | 0.638 | pyroglutamylglutamine | 0.607 |
| X-17438 | 0.638 | 3-hydroxy-2-ethylpropionate | 0.607 |
| X-13709 | 0.637 | palmitoyl-palmitoyl- | 0.606 |
| | | glycerophosphocholine | |
| 1-dihomo-linolenylglycerol (alpha, | 0.637 | gamma-glutamylleucine | 0.606 |
| gamma) | | | |
| 1-dihomo- | 0.636 | X-12822 | 0.605 |
| linoleoylglycerophosphocholine (20:2n6) | | | |
| N-acetylneuraminate | 0.635 | 1- | 0.605 |
| | | arachidonoylglycerophosphocholine | |
| | | (20:4n6) | |
| X-21668 | 0.633 | catechol sulfate | 0.604 |
| arginine | 0.632 | 3-methyl-2-oxovalerate | 0.604 |
| X-12117 | 0.631 | X-11538 | 0.604 |
| X-21410 | 0.629 | X-21729 | 0.603 |
| arabinose | 0.628 | X-21769 | 0.603 |
| 2-hydroxydecanoate | 0.625 | O-methylcatechol-sulfate | 0.603 |
| 2-arachidonoyl-glycerol | 0.625 | dopaminesulfate | 0.603 |
| X-17346 | 0.625 | tartronate (hydroxymalonate) | 0.602 |
| X-14427 | 0.624 | X-18913 | 0.602 |
| X-18938 | 0.624 | X-12100 | 0.601 |
| orotate | 0.624 | 1,5-anhydroglucitol (1,5-AG) | 0.601 |
| 3-ureidopropionate | 0.622 | tyramine-O-sulfate | 0.601 |
| X-21662 | 0.622 | 2-hydroxystearate | 0.6 |
| X-12472 | 0.622 | lactate | 0.6 |
| 1-arachidonylglycerol | 0.621 | N6-carbamoylthreonyladenosine | 0.6 |
| TL22:0 (behenic acid) | 0.621 | | |

The metabolite biomarkers were also used to derive statistical models useful to classify the subjects according to fibrosis stage 0-1 or fibrosis stage 2-4 using Random Forest analysis. Random Forest results show that the samples were classified with 74% accuracy. The positive predictive value, which is the proportion of subjects that were truly positive (i.e., subjects with fibrosis stage 2-4) among all those classified as positive, was 84%. The “Out-of-Bag” (00B) Error rate, which gives an estimate of how accurately new observations can be predicted using the Random Forest model (e.g., whether a sample is from a subject with stage 0-1 fibrosis or stage 2-4 fibrosis) from this Random Forest was 26%. The model estimated that, when used on a new set of subjects, the identity of fibrosis stage 0-1 subjects could be predicted correctly 54% of the time and fibrosis stage 2-4 subjects could be predicted 81% of the time.

Based on the Random Forest variable selection procedures, the metabolites that are considered reliably significant for construction of a model or algorithm for predicting fibrosis stage 0-1 or stage 2-4 were identified and ranked by importance. The metabolites that are the most important for distinguishing the groups according to this analysis are ribose, X-14662, isoleucine, I-urobilinogen, glutarate (pentanedioate), X-12263, X-19561, 2-aminoheptanoate, X-18922, gamma-glutamylisoleucine, X-12850, 1-arachidonylglycerol, X-17145, maleate (cis-butenedioate), malate, X-21892, N-methylproline, X-12739, X-21474, threonate, X-11871, glutamate, X-15497, 1-stearoylglycerophosphoinositol, X-21659, 3-hydroxyoctanoate, 3-methylglutaconate, X-14302, X-12812, and fumarate. All but four of the metabolites identified by Random Forest analysis (X-21659, X-21474, 3-methylglutaconate, and X-12812) had individual AUC values greater than 0.6096, the AUC for the clinical parameter Type 2 Diabetes.

Distinguishing Fibrosis Stages 0-2 from Fibrosis Stages 3-4

The performance of the clinical parameters for distinguishing fibrosis stage 0-2 from stage 3-4 were assessed by determining area under the curve (AUC) and logistic regression. The AUCs for the individual clinical parameters range from 0.5056 (Gender) to 0.6183 (Type 2 Diabetes) and the data are shown in Table 15. A total of 127 combinations of the seven clinical parameters are possible and all of the 127 possible combinatorial models derived using these clinical parameters were evaluated. The highest AUC was derived from a model that fit all seven clinical parameters, and the AUC was 0.6686.

TABLE 15
AUC values for select clinical parameters for distinguishing
fibrosis stage 0-2 from fibrosis stage 3-4 patient samples

| Clinical Parameter | AUC |
|---|---|
| Age | 0.5647 |
| Type 2 Diabetes | 0.6183 |
| BMI | 0.5043 |
| HDL Cholesterol | 0.6079 |
| Gender | 0.5056 |
| Fructose | 0.6009 |
| Past Alcohol Use | 0.5571 |

Logistic regression models and area under the curve (AUC) were also used to assess how well individual metabolites distinguished the fibrosis stage 0-2 samples from fibrosis stage 3-4 samples. Logistic regression analysis was performed on the measured values obtained for all 1151 metabolites detected in the samples. Sixty-one metabolites have individual AUCs greater than the AUC of 0.6183 that was obtained for the top clinical parameter, Type 2 Diabetes. Three metabolites (gamma-tocopherol, taurocholate, and xylitol) have an individual AUC greater than 0.6686, the highest AUC that was calculated from a model obtained using all seven of the clinical parameters evaluated. The data are shown in Table 16. All possible combinatorial models for separating fibrosis stage 0-2 from fibrosis stage 3-4 using these three metabolites (gamma-tocopherol, taurocholate, and xylitol) were generated. The highest AUC calculated when using a model containing all three metabolites was 0.7131 which is an improvement over the AUC 0 0.6183 using clinical parameters only.

TABLE 16
AUC of individual metabolites for distinguishing fibrosis
stage 0-2 from fibrosis stage 3-4 patient samples

| Biochemical Name | AUC | Biochemical Name | AUC |
|---|---|---|---|
| glutarate (pentanedioate) | 0.6349 | etiocholanolone-glucuronide | 0.6353 |
| epiandrosterone sulfate | 0.6303 | X-17453 | 0.6347 |
| androsterone sulfate | 0.63 | alpha-hydroxyisovalerate | 0.6335 |
| I-urobilinogen | 0.6656 | TL15:0 (pentadecanoic acid) | 0.6328 |
| 16-hydroxypalmitate | 0.6485 | 1-pentadecanoylglycerol (1- | 0.6303 |
| | | monopentadecanoin) | |
| fucose | 0.6522 | X-14658 | 0.63 |
| taurine | 0.6243 | X-12812 | 0.6297 |
| 3-hydroxydecanoate | 0.6279 | aspartylleucine | 0.6279 |
| 3-hydroxyoctanoate | 0.6205 | X-21408 | 0.6279 |
| X-11871 | 0.661 | cysteine-glutathione-disulfide | 0.6274 |
| X-12850 | 0.6542 | erythritol | 0.6261 |
| X-18889 | 0.6201 | 3-methoxycatechol-sulfate | 0.6249 |
| gamma-glutamylhistidine | 0.6229 | N-acetyl-aspartyl-glutamate | 0.6249 |
| | | (NAAG) | |
| taurocholate | 0.672 | X-14302 | 0.6245 |
| glycocholate | 0.6681 | glucose | 0.6244 |
| taurochenodeoxycholate | 0.6633 | X-13844 | 0.6243 |
| glycochenodeoxycholate | 0.6427 | cysteine | 0.6238 |
| xylitol | 0.6947 | X-18938 | 0.6237 |
| gamma-tocopherol | 0.681 | 2-hydroxy-3-methylvalerate | 0.6235 |
| tartronate-hydroxymalonate | 0.6651 | N-acetylmethionine | 0.6232 |
| 1,5-anhydroglucitol (1,5-AG) | 0.6635 | imidazole-propionate | 0.6229 |
| palmitoyl-palmitoyl- | 0.6634 | 2-piperidinone | 0.6203 |
| glycerophosphocholine | | | |
| cys-gly, oxidized | 0.6608 | 5alpha-androstan-3beta-17beta- | 0.6198 |
| | | diol-monosulfate | |
| X-14662 | 0.6597 | octanoylcarnitine | 0.6198 |
| X-11537 | 0.6577 | 3,7-dimethylurate | 0.6197 |
| oxalate (ethanedioate) | 0.6427 | delta-tocopherol | 0.6194 |
| threonate | 0.6399 | 7-methylxanthine | 0.619 |
| hypotaurine | 0.6383 | stearate (18:0) | 0.6185 |
| hydroxybutyrylcarnitine | 0.6377 | decanoylcarnitine | 0.6183 |
| mannose | 0.6369 | ribitol | 0.6183 |
| 1- | 0.6356 | | |
| pentadecanoylglycerophosphocholine | | | |
| (15:0) | | | |

The metabolite biomarkers were also used to derive statistical models to classify the subjects according to fibrosis stage 0-2 from fibrosis stage 3-4 using Random Forest analysis. The Random Forest results show that the samples were classified with 70% accuracy. The negative predictive value, which is the number of subjects that were truly negative (i.e. subjects with fibrosis stage 0-2) among all those classified as negative, was 79%. The “Out-of-Bag” (00B) Error rate, which gives an estimate of how accurately new observations can be predicted using the Random Forest model (e.g., whether a sample is from a subject with stage 0-2 fibrosis or stage 3-4 fibrosis) was 30%. The model estimated that, when used on a new set of subjects, the identity of fibrosis stage 0-2 subjects could be predicted correctly 81% of the time and fibrosis stage 3-4 subjects could be predicted 36% of the time.

Based on the Random Forest variable selection procedures, the biomarker compounds that are considered reliably significant for construction of a model or algorithm for predicting fibrosis stage 0-2 or stage 3-4 were identified and ranked by importance. The biomarkers that are the most important for distinguishing the groups according to this analysis are 1,5-anhydroglucitol (1,5-AG), glycocholate, I-urobilinogen, cys-gly (oxidized), taurochenodeoxycholate, taurocholate, 16-hydroxypalmitate, xylitol, X-12812, gamma-tocopherol, X-12850, fructose, X-14662, glucose, X-17453, fucose, mannose, glycochenodeoxycholate, X-11871, palmitoyl-palmitoyl-glycerophosphocholine, X-14658, imidazole-propionate, X-12093, X-14302, 2-hydroxyglutarate, X-12263, cysteine-glutathione-disulfide, tartronate (hydroxymalonate), aspartylleucine, and glutarate (pentanedioate). All but four of the metabolites identified by Random Forest analysis (fructose, X-12093, 2-hydroxyglutarate, X-12263) had individual AUC values greater than 0.6183, the AUC for the clinical parameter Type 2 Diabetes.

Distinguishing Fibrosis Stages 0-1 from Fibrosis Stages 3-4

To assess the performance of the clinical parameters (Age, Type 2 Diabetes, BMI, HDL Cholesterol, Gender, Fructose, and Past Alcohol Use) for distinguishing fibrosis stages 0-1 from stages 3-4 logistic regression and area under the curve (AUC) were performed. The AUCs for the individual clinical parameters ranged from 0.4939 (BMI) to 0.6698 (Type 2 Diabetes) and the data are presented in Table 17. A total of 127 combinations of these seven clinical parameters are possible and all 127 possible combinatorial models using these clinical parameters were evaluated. The highest AUC was 0.7217, and it was derived from a model that fit all seven clinical parameters.

TABLE 17
AUC values for select clinical parameters for distinguishing
fibrosis stage 0-1 from fibrosis stage 3-4 patient samples

| Clinical Parameter | AUC |
|---|---|
| Age | 0.6048 |
| Type 2 Diabetes | 0.6698 |
| BMI | 0.4939 |
| HDL Cholesterol | 0.6474 |
| Gender | 0.5302 |
| Fructose | 0.537 |
| Past Alcohol Use | 0.5254 |

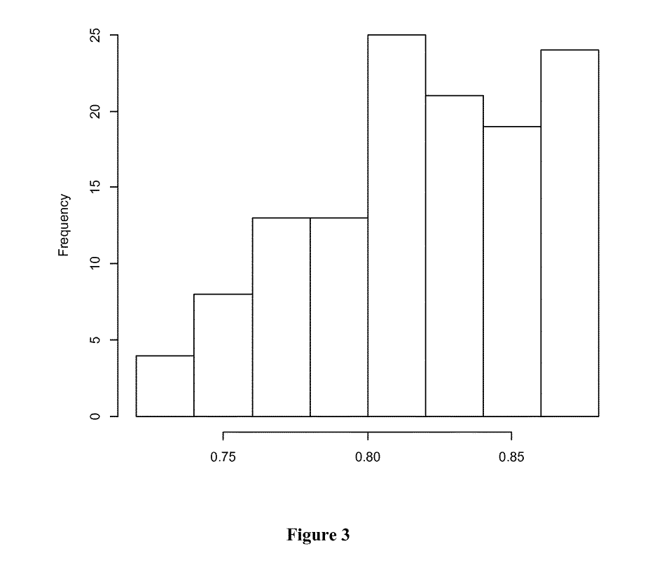

Logistic regression models and area under the curve (AUC) were also used to assess the performance of individual metabolites for distinguishing the fibrosis stage 0-1 samples from fibrosis stage 3-4 samples. Logistic regression analysis was performed on the measured values obtained for all 1151 metabolites detected in the samples. The analysis identified fifty-three metabolites with an individual AUC greater than 0.6689, which was the AUC for the top clinical parameter, Type 2 Diabetes. Seven metabolites (X-14662, I-urobilinogen, X-12850, glutarate (pentanedioate), xylitol, X-11871, X-11537) had an AUC greater than 0.7217, which is the AUC calculated from the model using all 7 clinical parameters of Age, Type 2 Diabetes, BMI, HDL Cholesterol, Gender, Fructose, and Past Alcohol Use. The data are shown in Table 18. All of the 127 possible combinatorial models for separating fibrosis stage 0-1 from fibrosis stage 3-4 using X-14662, I-urobilinogen, X-12850, glutarate (pentanedioate), xylitol, X-11871, X-11537 (the seven metabolites with an AUC>0.7217) were generated. The AUCs were calculated for each model, and the AUC from fitting all possible model combinations of the seven metabolites range from 0.7296 to 0.8788, and 89 of the models have an AUC greater than 0.8. The data is shown in FIG. 3.

TABLE 18
AUC of individual metabolites for distinguishing fibrosis
stage 0-1 from fibrosis stage 3-4 patient samples

| Biochemical Name | AUC | Biochemical Name | AUC |
|---|---|---|---|
| glutarate (pentanedioate) | 0.7407 | 1,5-anhydroglucitol | 0.6995 |
| | | (1,5-AG) | |
| epiandrosterone sulfate | 0.6833 | X-21892 | 0.6963 |
| androsterone sulfate | 0.672 | X-14302 | 0.6952 |
| I-urobilinogen | 0.7447 | X-22102 | 0.6947 |
| fucose | 0.7063 | 3-methylglutarylcarnitine | 0.6873 |
| 3-hydroxydecanoate | 0.6926 | X-18938 | 0.687 |
| 3-hydroxyoctanoate | 0.7138 | X-12263 | 0.6839 |
| X-11871 | 0.7328 | etiocholanolone-glucu- | 0.6836 |
| | | ronide | |
| X-12850 | 0.7429 | gamma-tocopherol | 0.6831 |
| X-18889 | 0.6889 | fumarate | 0.6825 |
| 16a-hy- | 0.6741 | N-acetylmethionine | 0.6823 |
| droxy-DHEA-3-sulfate | | | |
| gamma-glutamylhistidine | 0.669 | N-acetylcitrulline | 0.681 |
| taurocholate | 0.7005 | gamma-glutamylisoleucine | 0.6788 |
| glycocholate | 0.6984 | imidazole-propionate | 0.6778 |
| taurochenodeoxycholate | 0.6963 | ursodeoxycholate | 0.6762 |
| glycochenodeoxycholate | 0.6995 | palmitoyl-palmitoyl- | 0.6738 |
| | | glycerophosphocholine | |
| isoleucine | 0.6852 | X-12117 | 0.6735 |
| X-14662 | 0.7492 | 3,7-dimethylurate | 0.6733 |
| xylitol | 0.7381 | 2-hydroxy-3-methylvalerate | 0.6725 |
| X-11537 | 0.7307 | mannose | 0.672 |
| X-17453 | 0.7159 | X-14427 | 0.6714 |
| threonate | 0.7127 | hydroxybutyrylcarnitine | 0.6698 |
| malate | 0.7111 | X-12472 | 0.6698 |
| tartronate-hy- | 0.7106 | X-13529 | 0.6698 |
| droxymalonate | | | |
| X-21408 | 0.7016 | X-12802 | 0.6696 |
| X-14658 | 0.7011 | alpha-glutamyltyrosine | 0.6693 |
| oxalate (ethanedioate) | 0.7 | | |

### Example 5

### Lipid Metabolite Biomarkers of NASH in Human Serum

In another example, serum samples from 200 subjects spanning the spectrum of nonalcoholic fatty liver disease from NAFLD to NASH, including 181 subjects classified as having NASH and 19 subjects classified as not having NASH (i.e., the non-NASH subjects were classified as NAFLD or borderline NASH), were analyzed. Levels of metabolites, measured in μM, were determined in the samples using TRUEMASS complex lipid panel analysis.

The statistical significance and predictive performance of individual metabolites detected in the samples to determine the presence or absence of NASH in these subjects was assessed using logistic regression with Chi-square analysis and AUC calculations. Welch's two-sample t-tests were used to compare the metabolite levels in samples collected from subjects with NASH compared to the levels measured in samples collected from subjects without NASH. Logistic regression models and AUC assessed how well individual metabolites discriminated the NASH and non-NASH groups. Statistical analyses were performed using the measured values obtained for all lipid metabolites detected in the sample. The metabolites useful for distinguishing NASH from non-NASH patient samples are presented in Table 19. The Chi-square p-value is <0.1 and the AUC is >0.5 for all of the metabolites. Table 19 includes, for each metabolite, the lipid class of the metabolite, the metabolite name, the p-value determined in the logistic regression and Chi-square analysis of NASH samples compared to non-NASH samples, the AUC, and the direction of change (DOC) of the metabolite level in NASH samples compared to non-NASH samples.

TABLE 19
Biomarkers for distinguishing NASH
from non-NASH patient samples

| Lipid | | Chi-square | | DOC in |
|---|---|---|---|---|
| Class | Metabolite Name | p-value | AUC | NASH |
| CE | CE(24:1) | 0.011572 | 0.678 | Increase |
| PE | PE(P-16:0/14:1) | 0.01171 | 0.638 | Increase |
| LPC | LPC(14:0) | 0.021726 | 0.652 | Increase |
| SM | SM(18:1) | 0.023808 | 0.673 | Decrease |
| PE | PE(15:0/22:4) | 0.029071 | 0.625 | Increase |
| FFA | FFA(20:0) | 0.036282 | 0.629 | Increase |
| LPC | LPC(12:0) | 0.038773 | 0.607 | Increase |
| LCER | LCER(26:0) | 0.040104 | 0.637 | Increase |
| LPE | LPE(14:1) | 0.04582 | 0.614 | Decrease |
| PI | PI(16:0/16:0) | 0.058375 | 0.629 | Increase |
| LPE | LPE(20:4) | 0.064556 | 0.655 | Increase |
| DCER | DCER(20:0) | 0.065853 | 0.608 | Increase |
| LCER | LCER(14:0) | 0.067204 | 0.603 | Increase |
| PE | PE(15:0/18:4) | 0.068062 | 0.594 | Increase |
| PI | PI(18:0/16:1) | 0.068075 | 0.596 | Increase |
| PE | PE(16:0/22:2) | 0.06825 | 0.62 | Increase |
| PE | PE(P-14:1/18:1) | 0.068689 | 0.612 | Increase |
| PC | PC(16:0/14:1) | 0.070008 | 0.587 | Increase |
| PE | PE(18:0/17:0) | 0.076334 | 0.711 | Decrease |
| PE | PE(P-16:0/18:0) | 0.078532 | 0.617 | Increase |
| PE | PE(P-18:0/16:1) | 0.078833 | 0.61 | Increase |
| PE | PE(O-18:0/18:0) | 0.080522 | 0.597 | Increase |
| CER | CER(26:0) | 0.080756 | 0.63 | Decrease |
| PE | PE(16:0/16:0) | 0.082878 | 0.6 | Increase |
| LPE | LPE(18:4) | 0.085339 | 0.569 | Increase |
| PE | PE(O-18:0/14:1) | 0.090197 | 0.592 | Increase |
| LPE | LPE(18:2) | 0.08793 | 0.627 | Increase |
| LPE | LPE(20:3) | 0.069501 | 0.61 | Increase |
| PE | PE(14:0/14:1) | 0.016016 | 0.643 | Decrease |
| PC | PC(14:0/22:4) | 0.019756 | 0.647 | Increase |
| PC | PC(15:0/16:1) | 0.077462 | 0.632 | Increase |
| PC | PC(20:0/14:1) | 0.094651 | 0.595 | Increase |
| PC | PC(17:0/22:6) | 0.049212 | 0.635 | Decrease |
| PE | PE(15:0/18:3) | 0.042745 | 0.615 | Increase |
| PE | PE(17:0/20:2) | 0.000767 | 0.716 | Increase |
| PE | PE(18:2/20:2) | 0.012908 | 0.623 | Increase |
| PE | PE(18:2/20:3) | 0.060489 | 0.623 | Increase |
| PC | PC(18:1/22:6) | 0.006359 | 0.692 | Decrease |
| PC | PC(18:1/22:5) | 0.023452 | 0.648 | Decrease |
| PC | PC(14:0/18:4) | 0.04088 | 0.585 | Increase |
| SM | SM(16:0) | 0.043787 | 0.666 | Decrease |
| CE | CE(24:0) | 0.063854 | 0.609 | Increase |
| PC | PC(14:0/20:2) | 0.06602 | 0.625 | Increase |
| PC | PC(14:0/20:3) | 0.067265 | 0.634 | Increase |
| PC | PC(18:1/18:4) | 0.067417 | 0.602 | Increase |
| SM | SM(18:0) | 0.073073 | 0.659 | Decrease |
| PC | PC(14:0/18:2) | 0.075939 | 0.602 | Increase |
| PC | PC(14:0/16:1) | 0.093209 | 0.587 | Increase |

### Example 6

### Lipid Metabolite Biomarkers of Fibrosis in Human Serum

In another example, serum samples from 200 subjects spanning the spectrum of nonalcoholic fatty liver disease from NAFLD to fibrosis, including 150 subjects classified as having fibrosis and 50 subjects classified as not having fibrosis (i.e., the non-fibrosis subjects were classified as having NAFLD, borderline NASH, or NASH) were analyzed. Levels of metabolites, measured in μM, were determined in the samples using TRUEMASS complex lipid panel analysis.

The statistical significance and predictive performance of the individual metabolites detected in the samples to determine the presence or absence of fibrosis in these subjects was assessed using logistic regression with Chi-square analysis and AUC calculations. Welch's two-sample t-tests were used to compare the metabolite levels in samples collected from subjects with fibrosis compared to the levels measured in samples collected from subjects without fibrosis. Logistic regression models and AUC were used to assess how well individual metabolites discriminated the fibrosis and non-fibrosis groups. Logistic regression and Chi-square analysis was performed using the measured values obtained for all lipid metabolites detected in the sample. The metabolites useful for distinguishing fibrosis from non-fibrosis patient samples are presented in Table 20. The Chi-square p-value is <0.1and the AUC is >0.5 for all of the metabolites. Table 20 includes, for each metabolite, the lipid class of the metabolite, the metabolite name, the p-value determined in the logistic regression and Chi-square analysis of fibrosis samples compared to non-fibrosis samples, the AUC, and the direction of change (DOC) of the metabolite level in fibrosis samples compared to non-fibrosis samples.

TABLE 20
Biomarkers for distinguishing fibrosis
from non-fibrosis patient samples

| Lipid | | Chi-square | | DOC in |
|---|---|---|---|---|
| Class | Metabolite Name | p-value | AUC | Fibrosis |
| CER | CER(14:0) | 0.00463317 | 0.629 | Decrease |
| DCER | DCER(14:0) | 0.006578011 | 0.632 | Decrease |
| LPE | LPE(12:0) | 0.007391435 | 0.56 | Decrease |
| DCER | DCER(18:0) | 0.008371679 | 0.616 | Increase |
| PE | PE(18:0/22:2) | 0.011506569 | 0.639 | Decrease |
| PE | PE(P-18:0/18:3) | 0.013343082 | 0.591 | Decrease |
| LPC | LPC(17:0) | 0.015657688 | 0.645 | Decrease |
| LPC | LPC(22:0) | 0.019213085 | 0.565 | Decrease |
| CER | CER(18:1) | 0.021646673 | 0.598 | Decrease |
| LCER | LCER(22:0) | 0.027909317 | 0.598 | Decrease |
| PE | PE(16:0/20:1) | 0.028130909 | 0.575 | Increase |
| CE | CE(15:0) | 0.028252905 | 0.592 | Increase |
| PE | PE(16:0/22:4) | 0.032648353 | 0.58 | Increase |
| PE | PE(O-18:0/20:2) | 0.035331647 | 0.575 | Increase |
| LPC | LPC(20:0) | 0.035456326 | 0.558 | Decrease |
| LPE | LPE(24:0) | 0.037831237 | 0.57 | Increase |
| PC | PC(12:0/14:1) | 0.044802369 | 0.626 | Increase |
| PE | PE(17:0/22:2) | 0.046039384 | 0.529 | Increase |
| SM | SM(18:1) | 0.047038106 | 0.584 | Decrease |
| CER | CER(16:0) | 0.053022252 | 0.577 | Increase |
| LCER | LCER(24:0) | 0.05496295 | 0.579 | Increase |
| PE | PE(O-18:0/20:3) | 0.056006144 | 0.572 | Increase |
| CE | CE(17:0) | 0.058289914 | 0.575 | Increase |
| PE | PE(P-16:0/18:3) | 0.063729692 | 0.583 | Increase |
| PE | PE(P-16:0/16:1) | 0.06392523 | 0.591 | Increase |
| LPE | LPE(14:1) | 0.065098016 | 0.546 | Increase |
| FFA | FFA(24:0) | 0.066918972 | 0.583 | Increase |
| PE | PE(O-16:0/18:4) | 0.070807569 | 0.515 | Increase |
| FFA | FFA(15:0) | 0.072330933 | 0.565 | Increase |
| SM | SM(14:0) | 0.073276682 | 0.606 | Increase |
| LPC | LPC(20:2) | 0.075129487 | 0.586 | Increase |
| PE | PE(P-14:1/18:1) | 0.077182728 | 0.574 | Increase |
| SM | SM(24:1) | 0.083433905 | 0.686 | Increase |
| PI | PI(18:0/20:2) | 0.087177268 | 0.59 | Decrease |
| LPC | LPC(15:0) | 0.088071945 | 0.61 | Increase |
| PE | PE(O-18:0/18:1) | 0.08819528 | 0.591 | Increase |
| PI | PI(18:1/20:3) | 0.089568234 | 0.564 | Increase |
| PE | PE(16:0/16:1) | 0.090266554 | 0.563 | Increase |
| DAG | DAG(18:1/20:3) | 0.094383206 | 0.548 | Increase |
| PE | PE(18:2/20:2) | 0.010797664 | 0.606 | Decrease |
| PE | PE(14:0/16:1) | 0.011977102 | 0.615 | Decrease |
| PE | PE(14:0/14:1) | 0.024831761 | 0.578 | Increase |
| PE | PE(16:0/18:1) | 0.039427836 | 0.566 | Increase |
| PE | PE(18:1/18:1) | 0.059872921 | 0.582 | Decrease |
| PE | PE(17:0/20:4) | 0.061927251 | 0.571 | Decrease |
| PE | PE(14:0/20:5) | 0.067287806 | 0.546 | Increase |
| PE | PE(16:0/22:5) | 0.070517233 | 0.551 | Increase |
| PE | PE(18:2/20:3) | 0.070676672 | 0.573 | Increase |
| PE | PE(16:0/20:4) | 0.074163743 | 0.555 | Increase |
| PE | PE(14:0/18:2) | 0.076434889 | 0.564 | Increase |
| PE | PE(18:1/18:4) | 0.078563589 | 0.552 | Increase |
| PE | PE(15:0/22:6) | 0.07868741 | 0.531 | Increase |
| PE | PE(16:0/14:0) | 0.092319032 | 0.523 | Increase |
| LPC | LPC(18:3) | 0.097780133 | 0.563 | Increase |
| TAG | TAG55:7-FA20:3 | 0.005158253 | 0.64 | Decrease |
| TAG | TAG53:6-FA18:2 | 0.006286965 | 0.655 | Decrease |
| TAG | TAG55:7-FA20:4 | 0.009926564 | 0.633 | Decrease |
| TAG | TAG53:5-FA18:2 | 0.010360485 | 0.646 | Decrease |
| TAG | TAG53:7-FA18:3 | 0.010758415 | 0.624 | Decrease |
| TAG | TAG55:8-FA20:4 | 0.011202368 | 0.6 | Decrease |
| TAG | TAG53:5-FA18:1 | 0.011522457 | 0.635 | Decrease |
| TAG | TAG55:6-FA20:3 | 0.011575067 | 0.6 | Decrease |
| TAG | TAG57:9-FA22:6 | 0.01204974 | 0.607 | Decrease |
| TAG | TAG53:6-FA18:3 | 0.013100706 | 0.633 | Decrease |
| TAG | TAG55:6-FA18:1 | 0.013897539 | 0.6 | Decrease |
| TAG | TAG53:6-FA18:1 | 0.014634314 | 0.555 | Decrease |
| TAG | TAG53:4-FA18:1 | 0.017988499 | 0.633 | Increase |
| TAG | TAG53:4-FA18:0 | 0.028729279 | 0.586 | Decrease |
| TAG | TAG51:4-FA16:0 | 0.032253584 | 0.617 | Decrease |
| TAG | TAG53:3-FA18:0 | 0.035029698 | 0.538 | Decrease |
| TAG | TAG51:3-FA16:0 | 0.057202962 | 0.603 | Increase |
| TAG | TAG51:4-FA18:1 | 0.071294284 | 0.627 | Increase |
| TAG | TAG56:5-FA20:4 | 0.081720387 | 0.532 | Increase |
| TAG | TAG56:5-FA18:0 | 0.083535689 | 0.536 | Increase |
| TAG | TAG56:4-FA20:4 | 0.090401136 | 0.554 | Increase |
| PE | PE(14:0/18:1) | 0.021633768 | 0.582 | Decrease |
| PC | PC(14:0/18:4) | 0.030623649 | 0.558 | Increase |
| PC | PC(18:2/22:5) | 0.03350978 | 0.599 | Increase |
| PC | PC(20:0/22:5) | 0.048095626 | 0.576 | Decrease |
| SM | SM(18:0) | 0.051143938 | 0.587 | Increase |
| CE | CE(18:0) | 0.070495066 | 0.529 | Increase |
| PC | PC(18:2/18:4) | 0.071768872 | 0.561 | Increase |
| PC | PC(14:0/20:2) | 0.081193509 | 0.606 | Increase |

1-37. (canceled)

38. A method of diagnosing or aiding in diagnosing whether a subject has liver disease, comprising:
analyzing a biological sample from a subject to determine the level(s) of one or more biomarkers for liver disease in the sample, wherein the one or more biomarkers are selected from Tables 12, 2, 3, 4, 5, 7, 8, 10, 11, 14, 16, and/or 18, and
comparing the level(s) of the one or more biomarkers in the sample to liver disease-positive and/or liver disease-negative reference levels of the one or more biomarkers in order to diagnose whether the subject has liver disease.

39. The method of claim 38, wherein the liver disease is NAFLD and the one or more biomarkers are selected from the group consisting of 5-methylthioadenosine (5-MTA), glycine, serine, leucine, 4-methyl-2-oxopentanoate, 3-methyl-2-oxovalerate, valine, 3-methyl-2-oxobutyrate, 2-hydroxybutyrate, prolylproline, lanosterol, tauro-beta-muricholate, and deoxycholate.

40. The method of claim 38, wherein the liver disease is NASH and the one or more biomarkers are selected from Tables 7, 8, 10, and/or 11.

41. The method of claim 38, wherein the liver disease is fibrosis and the one or more biomarkers are selected from Tables 12, 10, 11, 14, 16, and/or 18.

42. The method of claim 38, wherein the diagnosis comprises distinguishing NASH from NAFLD.

43. The method of claim 38, wherein the diagnosis comprises distinguishing NASH from fibrosis.

44. The method of claim 38, wherein the sample is analyzed using one or more techniques selected from the group consisting of mass spectrometry, ELISA, and antibody linkage.

45. The method of claim 38, wherein the method comprises analyzing the subject and a biological sample from the subject using a mathematical model comprising one or more biomarkers or measurements selected from Tables 12, 2, 3, 4, 5, 7, 8, 10, 11, 14, 16, and/or 18.

46. The method of claim 45, wherein the mathematical model is used to generate a Liver Disease Score wherein the Liver Disease Score is selected from the group consisting of a NASH Score, a NAFLD Score and a Fibrosis Score and the Score is used to aid in the determination of the presence or absence of liver disease in the subject.

47. A method of determining the fibrosis stage of a subject having liver fibrosis, comprising:
analyzing a biological sample from a subject to determine the level(s) of one or more biomarkers for liver disease in the sample, wherein the one or more biomarkers are selected from Tables 12, 2, 3, 4, 5, 7, 8, 10, 11, 14, 16, and/or 18, and
comparing the level(s) of the one or more biomarkers in the sample to high stage liver fibrosis and/or low stage liver fibrosis reference levels of the one or more biomarkers in order to determine the stage of the liver fibrosis.

48. The method of claim 47, wherein the method comprises analyzing the subject and a biological sample from the subject using a mathematical model to determine the liver fibrosis stage of a subject having liver fibrosis.

49. The method of claim 48, wherein the mathematical model is used to generate a Fibrosis Score and the Fibrosis Score is used to determine the stage of liver fibrosis in the subject.

50. A method of monitoring progression/regression of liver disease in a subject comprising:
analyzing a first biological sample from a subject to determine the level(s) of one or more biomarkers for liver disease in the sample, wherein the one or more biomarkers are selected from Tables 12, 2, 3, 4, 5, 7, 8, 10, 11, 14, 16 and/or 18 and the first sample is obtained from the subject at a first time point;
analyzing a second biological sample from a subject to determine the level(s) of the one or more biomarkers, wherein the second sample is obtained from the subject at a second time point; and
comparing the level(s) of one or more biomarkers in the first sample to the level(s) of the one or more biomarkers in the second sample in order to monitor the progression/regression of liver disease in the subject.

51. The method of claim 50, wherein the method further comprises comparing the level(s) of one or more biomarkers in the first sample, the level(s) of one or more biomarkers in the second sample, and/or the results of the comparison of the level(s) of the one or more biomarkers in the first and second samples to liver disease-positive and/or liver disease-negative reference levels of the one or more biomarkers.

52. The method of claim 50, wherein the method comprises analyzing the subject and a biological sample from the subject using a mathematical model comprising one or more biomarkers or measurements selected from Tables 12, 2, 3, 4, 5, 7, 8, 10, 11, 14, 16, and/or 18.

53. The method of claim 52, wherein the mathematical model is used to generate a Liver Disease Score wherein the Liver Disease Score is selected from the group consisting of a NASH Score, a NAFLD Score and a Fibrosis Score.

54. The method of claim 53, wherein the Liver Disease Score is used to monitor the progression/regression of liver disease in the subject.
